SEQUENCE LISTING (332 sequences)

SEQ ID NO: 1 - DNA, 93000 bp, Homo sapiens
tctctaattagccaggcatggaggtgcatgcctgttgttccaggtacttgggaggctgagacaggaggattgtctgagctcaggagctcacaccactgtactacagcctgggtgacacaatgagaccttgtctcaaaaaaatagaaacatgcaagaggtaaccatgtattgtttgctaaggagcatgtggtaaactataaggaaaaggaagaaaatgaaaaacaaaatttgtgatagttcttccatctgcagggaaggattatgagattggggagaggcacatgggggtcttttattgtgatggtatttttttttttttttgagacggagtctctctctgtcacccaggctggagtacagtggcccgatctcggctcactgcaagctccgcctcccggattcacgcctttctcctgcctcagcctcctgagtagctgggaatacaggcgcccgccaccacgcccggctaattttttgtatttttagtagagacgggttttaccgtgttagccaggatggtctcgatctcctgaccttgtgatcaccccgcctcagcctcccaaagtgctggtattataggcgtgagccaccgagcccggtcgatggtaattttttttatccaagcctgctcctcatagtttatctactcattgatgtttaataaaacatcaatgttttattgattatcaatgtttaataaaacatctactcattgatgtttaataaaaatttaagaccaggcatggtggctcatgagcctgtaatcccaacattttgggaggctgagacgggaggatcacttgagcccaggagtttgagacagcctgtgcgacatggtgaaactctgtctctacaaaaaatacaaaaattagctgtgctgatttttttaaaaatttaaagcagtgacagatacaaacatggttactattaggcattaagattacaactggggccaggcgcggtggctcattcgtgtaatcccagcattttgggaggccacggtgggtggatcacttgaggtcaggagtttgagaccagcctggccaacatggtgaaaccccatctctacaaaaaatacaaaaaaattagctggatgtggtggcgagcgcctgtaatcccagctactcaggaggctgaggcaggaagatcacttggaggttgaggttgcagtgaactgagatggtgccactgcactccagcctgggccacagattgagactacacctccaaaaaaaaaaaaattacaactggtttcacatgttaactcatgtaatacctccagcaaccttgtgaggcaggacttgttatcatattcattctacaggtgcggaaactgaggcacgaggaggtcacaagacctgcccaaggccatgcttgtgatgagagacagagtttggttttgaacaatggcctcactcaactccatgcacattctgcatttatgaagctctttcatgtgggctgggggcaggagaccctgtggggctcagcctttcatgctagtttaggtgccagtgatacagcgagcaggttgcagggcaaggggcccacatggagctgcccagtttgatatgtggggagggggccaggcgcagtggctcaggtctgaatcccagcactttgggaggccgagatgggcggatcccctgaggtcaagtgttcaagaccaggctggccaacatggtgaaacctcgtctctactaaaaataaaaaaaataaaataaaaaaaaaaagctgggcatggtggcatgcgcctgtagtcccagctactcggggaggctgaggcaggagaatctcttgatcccaggaggcagaggttgcagtgagccaagattgcgtcactgcattccagcctgggctgcagagggagactccatctaaaaaaaagaaaagtggggaggagtggcagagtttcgttcgggtcagggcccaatcagaggaagggataggggctgggggagtccagagaggagaaacaaaggctcagcttggggcagtcagggaagccttcctggaggagaggatattcagacaagacattgatggatgaaaaggagttcagagtgtgggcaaggacccatgggctggatggcttagagacatagagagagacctagacaggatgcagtagagctgcaaaaagactttacagagagaggctaagagacacatgactgagagagcagtggacagagtcagaggaacagagacccagggagagaaagagaccaggcagtagagttagagagacagagacgtagagacagaggaagagagatactcagagaacaggaccatctgcccggtgctggtgccgggcgtacacacaagagcttgcatttgcctgtacttgcttgagtattgcagggaggcgtttgcccaggttaccagggcaggaatgtccatgcatgggtggcgggggcacgtgcaccatttcagtgcaggtgagggtctgcacaggaggaaacctgcttgggggccgccgtcgccccttgtccccctgcaacgaggaggtgtggggcgaggcgccccttcccggagccaggccctttaacgcccccccagcccgcagcgcccccgccccatcaggcgtccctgggctccgcctgcccgccccgcatcagcgccacgtcaggcgctgtccgcgcccccggtgctgaaccaagatggccggtggcggccgggccccggcgtgagccaagcgcgggctgcagccgggagatgccccagcccagcggccgctgagcccgacccgacagagccggcccggccgcctccggcccacctgcgagctcggagacatgtctctgctttgcgttggaggtaggaggcgaggctgggggtcgggggacccgtgtggacgcgggctggggtcggggtccgcgtggatgcggctcccagggggttcctggggggtgacccgagtccaggtggctggacttttgagaggggtagcccttgtcgacgcagctgggccccggagggggtgatccgtgtagacgcggcagggctctcgggggtcctggtggccgctgctggaggggtcgtgtggacgcggctgaacgcggaggcccgtgtggacgtggacgggtatccggggttctacccggaaacgatctagactggggcgtctgtgtagaccaggtttttagagggtcagtgaggaagagtctgggctcggtagtttgattgaagcctgcaggggtgcttggaaggggtctggtgttgtgggctgggttacgggtcctcatggacgcggacccggctggaaattcgaaggagagtgcggtgggagggttctccaagccgtggggatgtaagatataggtagaggggcttggccatcagggcatccacgagttctaggactctcccagccctaggtgtaaagaggactggagtggggggagtgtggagacaggagacccctcccagcgtccagagagggctgtgactggaggtctgggggcgcttttccagttaactctttggcctcccgagtgagggggttggaggaatgtccagaaggtcttcccgctccctgccccctccggttctcccaaacacgtttcagggccttctcggcagggagcctacttcttggaagagcaatgtctcctggtccccctcctggcccatcctcacctcacccactttgcactggagagagggagggagggagacgtgaggtggggggggagctattttggggccagaacgccatctgcacagaggcccacgcccaggcccacgctctgcgttggggttggaggagggagggagacgtgggaggggcatgaccccccaccccacacctttatggctctggggtcgtggggaagccagcccaaccgaacccaggcgttctgggcactcccgtttgagaaatcaaactctgggagaccttgggcggcaggggtggatgtggaccgccgttccacgcctacagagctctgaacccctggttcttaaggactggggtccccccgtcactcccacccatttcaagaaggctggggtgggggaaggggtgtgtctgccaagcctgagactgcaggcttcgggagacctcgggcgacaggaagcgtgcgtgtgctgaactcctgctgcctggacacagggtgggcaggccggggagccgggaaccgcccccgccgcccctcctcctccctcccctccccctcggccgattcattcacaaaccctgccccgcccgcccgctccccgccgcggcagcttccgagtgcaaatccggccgggctgggccggcgtggacgcgggtgttcttaaagggacaggggcctactttctctaagctcgtttctgctggacggggctgggggtggtggaagaagggtccctagagtcaaaggtcaactcctccatcccctagcctccaggcctatggagttaggggagtagggaagtgaaggagacccctaggcgacccccaccgcctaggcctgaggacggaatgggaggagaacttgaatgtccagaccccctactgacctctccccctgctctaagtagccgggctcttaaaggtggcactggaggaggttccgacgtgggtgttcttggctgactcctctatcctctctcttctccccccagcatcccgccccaccccgcctcacatcctccaagttgggcttctctggaaggtttgcctagtggagtgggggggggggggggggggcggggatctcacccttaactctgacgcaaacaggaggctcggctgcgggggtggcattaagggggggcagcaggaggcaagagggatgaggcttccagaaacagagacgttgggggaggactgtgctggattctgtggactttacagggaggggcccggctgagtggagaggaatcgctgggaggggcaaggctggggaagaggttgacaggggtgggaggtaaggccctcaggtgtaaagggaggtgtgaggttggagtgaggaggagtaggaggcagggaggggtggggagagggagaagtgctgggtgaggccagtctcccagggagaggtgggatggtgcccagagaggggaggggctgggatggctgagcacgagagatgttggggtgggcttggcaggagtgaagctaggctgagatgggttgccgtgttgcctgagctcagctgaggaaatacctgtcccccctcccctccaccggggacccacaggcgtttgccaatccctacacacattcatacccagacttgttcctgtgtggacacaaatgcttaggatgtatgaaggctctacgcagaccatgtgcctgggaaggcacattccgtaggcacacgtgtgtactcaaatgcctgtgtgaacacaagcatacacgtgtatataggacacagtctgccgctacagatatagtcccagcaacacacgtgtgagtgtgtgtgtctgccagcacgcatatagatgtatccacgtgtctgatgaatgctcacaatatacatgtcacataaagatccacccatgtccagccgagcgcggtggctcaggcctgtaatctcatcactttgactttgggaggccaaggtgggcagatcacttgaggtcaggagttcaagaccagcctggccaacatggtgaaactttgtcactgctaaaaaataaaaaaaattagccaggtgtggtggcgcgcacctgtagtctcagctactcgggaggctgaggaaggagaatcacttgaataggggaggtggaggttgcagtgagctgagatcctgccactgtactccagcctgggcaacacagtgagactctgtctcaaaaaaaaagattcactcatgtcctaacactcagacatgtacccatacatatctgttaaggtccccaaccccatgaagcacatatatattcatataagcacatgtcaaagccatgtgtgtatgcacatgacaacacttgcctggacattcaaagcagcccctttcaacaagtggatgatgatgcatacaagtaaatacacatttgagtatttcattgcacacaagtgcatacatgtatgcagataaacaaatacagctgccacagaactgcatccatgtacacatgcaaacatcaatatgtgtgttttccacctaacatatcacccacaagctcacacacaccagtgtgtctctgatgtagtgacaccccactcctagacacacatgtgcagactcccacctgcacttaaatggtgcacatactctgcacacatgcacactcatatccatgtagaggcaggcacctgcctgccctggtaagatgagccaactactgctgcaaatacccaagcaagtacaggcaaatgcaagctcttgtgtatacacccagcaccaacacatagcagaccaccctgtatggtgtatgggccttatctctgcccgcttcatttatttattcatgtggtgccccgctgcttcccaacatcgtaatccactgactaggtcttggcaggatcctgaggctgtgaatggtaaatgccacctggttccttcatgaagtcacttattttatttatgcaacaagcattaattgagcacctactgtataccaggcactgttcctggctctgggacccagccctattcctagccatcactcctgcacgtcccatggggttagaatgaaagagatgagggagggctaagggagctgaggagtggggtatccagggctctcttcctctgggaagcctaccctgattccaccaccaccatttacttgagtctcagaagatgtgatgagggggagatgagcaggagggagatggtgctctgggtggagggaacagcatgagcaaaagcatggtggtgtgaagggttacttccagatcttcctatgtgcctcacagcattttgcacaggccacttgatgttttgttttgttttgagacagagtctcactcaaagcctgttactcaggctgcagtgcagtggcatgatctcagcttgctgcagccttgacctccagggctcaagtgatcctcctgcctcagcctcccaagtagctgggagtataggcatgcaccaccacacccggctgacttttttctttttaattttttgcagaagcagggttttgccctgctgcccaggctggtcttgaactcctgggctcaagtgattcaccctcctcagcctcccaaagtgctaggattacaggtgtgagccaccacgcccaactgatgttttaaaggctctataaacatgttatgaataaatgacatgaacaaatcagtgaatttaggtgagggtgggtttgcttcacaacttacaataatatcaacaacaaaccccatttatccagcttttttgtttgtttgtttgttttttgagacacggtctcactctgtcacccggactgcagtgtagtggtgtgatctcggttcactgaaacctccaccttccaagttcaagcgattttcctgcctcagcctcccgagtagctgggattacaggcacgtgctaccacacctagctagtttttgtatttttagtagagatggggtttctcatctctactaaaaatacaaatacatctctagtggccaggctggtcttgaactcctgacttcaagtgatcctcttgcctcggcctcccaatgtactgagattacaggctcgagccaccgtgcccagcccagcatttcataggcatgatgttaaggatttttatgcccatgccctctttaactcttacagtagccccattgtacagatgaggaaaactgaggctcagggagctttaacaacttgcctggtggaacagagccagaggtggagctgactgactctagagcctactggggttggcagaagtttgagccttctctcagaccccaggacctcggagacagccaagtcccttctgtggagtgatgttctgccctgggattgccttcttctttctatttatttatttattggagacagagtctggctctgttgcccaagctggagtgcagtggcacaatttcagttcactgtaacctcctcctcccgggttcaagcaattctcctgcctcagcctcctgagtagctgagattacagatgcacaccatcatgcccagctaattttttgtatttttagtggagatggggtttcaccatgctagccaggctggcctcgaactcctgaccaccaagtgatctacctgctttggcctcccaaagtgctgggattacaggcatgagccactgtgcccagccaggattgctttcttctctcatgcatctagtaaacagacatgtcacaatctctccattcatcggggctattcccctgctccttgcctaagaagccttcacacatccctccgttcctggctactcagtccctgataccctccagctaggaggagctgcatctaggtcttagctcctaccgttctcatgggatgccctccagagttgcatcttcagggctgcagcctaaccctttccctaacctggcctcacttttcttccacatacaatggacctcttctgcaaggtgccaataagggcatcctgggtccagcaggcctgagtgtgaattgctaagtccaccacttcctggcaagtctcacatcctctgcattccttggtgtgctcatctgtggaatgggcacaggagtagcatccaccatctacgtttgccagaactctctaatgaggcttgcagagcccctggcatgactcctggcctacgggaaggcccaataatcgtgagcaaaagacatgaaaaatcaatgatgggccggccccggtggctcacgcctgtaatcccagcactttgggaggcctaggcgggcagatctcctgagctcaggagttcaagaccaccctgggcaacatggtgaaaccctgtctttactaaaatacaaaaaaattagctgggtatggtggcgcgcgcctgtagtcccagctactcagggggctgaggcacgagaatcgcttaagccccgaaggcggaggttgcagtgagccgaggtcgcgccactgcactccagcttgggctacagagtgagactccgtcaaaaaaaaaaaaaaaaaaaaatcaatgatgatgtttttattaatggagactaaaagctttctggaaggcggttctgagtgcaggacatttctgagagtccactgcattctggggcccgaggagcgctccaagaggggtcccccagggccactccaggagggagcctagttcgttgagggtacacgggtgggaccgagcattcagcaccgtggacagcgcctgcccgcgcctctgggggacagctcttagctcagcctcccaggattaatggaggaggtgacgtcattccttccgtgaattactatttaatgagcacctactataggctaggcctacttctaagtggttcatggtaaacaacacacacacaaatcccttattcccgaagctctctggaactccaggttcttggtgttttttacactgaaggctcagtctggctctggtctatttgtgtcaacggaggataagggtcctcatttagtgtctcccagatgcgaaaccagcacagctgaggtcttttatcctaaagcccatccatctcccgctgaaatgccctccaccaacatcacacacaaaacgatcaaatctcctataatctatttctggattctctatgcttcctcccttcccccaagctaatcctggggcgggtgggctttcagaagccctagacggctggatctccacctcctgtctccccaccccatcccacgtggcaggtcagggagccagtgccccaagactgcagtcccccctaattagaatccagggtcagtcagccatgtggttcacgcctgtaattccaacactttgggaggccgaggggatggcttgagccttgaagccagccatagcaacatagcaagaccccatctctacaaaaaaaaaaaaaaaaaaaaaggaatagccaagcatggtgatgcgcacctgcagtctcagctactcgagaggctgagtggggaggatcgcttgaacctgggagatcgaggctgcagtgagccatgatcacaccactgccctccagcctggagcctgggcaacagagagagaccctgtcaaaaaaaaaaaaaaaaaaaaaaagaaaagaaaagaaaaaaaaagaagaagaagaagagaaaagaaagaaaaagaaaagaatccagggtattcaaatcctagaatccaggccagggacacctttagaacacaaccaaataataaaaacagtaaagatatcagtaataataataatagtataaatgacactataatcattctgcagaaggggtgagggaggaagaggagggaagaagaggaggagaaaggagagggaagggagatcagccactccgtctccccattttccagatgcagaaacggaggcgcgaagccaggctgcaggggtgtcccgcagcctgaactcctgcctcagcctccccgggtacggccactacattcatcagtcagcgcttctgggggccccaaagacctttgaggccgcatgacgcggtgtcttcacccgctcgtcgctggggacagcagtgggcacccccctcatccccgcccccctgtccgccggcgcgcctcccacgccctccgcgcgcccacgctataaatagcctcatgcgtgtgacccgaagcccgctggggaagggtggcgcttccgccccgctgaccctgatacagcaacccggcgcaggcggggaggtggcgggatgtgacgacgaagacggtgactcagggaccatttactgaccggggaagacagggagagagactgggttccttctctgagcccccttcctcctccaagcctctcccccgataccaccctcccctctgcgcccatcccccttcccgaatcccctccctcaccgcgaacacccttccctcctgagaaccccctcgccactctcggccccctttcctgtccccttttccctctgagacccctctcccactgggagccccagtgatgaaaaatcatggatgtcctcaacctggacctcggcagggagctaagtgcaggagcctggcttcagctatcagagggtacagccctccagccggtcccccactgcctgaggaccccaacggagaccattcccctctagccaagagaggctgcaggaaggagctgaggcttagggaagaggagccggactgaccaagggaggggcacaagaagggcagagacagtgagagagagaaggagagaacagagaccaaaagaagacgaccagagtggagagaaatagaacggggaggagggagatgagagacagggattgggaaagagagagagagaacagatcaaggagctactgggctccccgctgagcctaaatccatggtttataaaacagggcacagttggaggagcttcaagtgagcccagcccagtctagaccccagtgagccccttgtatcaccccccagagcctgtcactttcccacccccatatgcatccctacttttctcccccaactgcctctcctctccccagtgtctcctccacctgcctgccctccctagtggaaatctttccccctctctcttttattattattattattattatatattttttgagacagagtcttgctctgtcgcccaggctggagtgcaatggcgtgatcttggctcactgcaacctccgcctcctgggttcaagcgattctcctgcctcagcctcccgagtagctgggactacaggcgtgcaccatcatgcccggctaatttttgtatttttaatagagatggggttttaccatgttgcccaggctggtctcgaactcctgacctcatgtgatccacctgcctcggcctcaaagtgctgggattacagatgtgggctaccacgcccagcctctctcccttttaaattattatttttagacatgggatcttactacattgcccaggctggtcttgaactcctgggctcaagggatcctcctgccttagccttcctagtagctgggattacaggcatgtgcctctatgcctggtactctcgctgtctccttgtgtctctctatttctctccatctgtccagactctcctctcttgctttcatgcccatcatttcttcctctctcctctcccttcctctccctgtatgagtccctccagctgtgggtgcctttctcagcctctgtcagcatcccctccacccccgatcccgtcacattctacctcttgccccttcccctgcccacccccttcacagagagtccccacgcctgcaggctggacctcctccaggagccaggcccaaagctcctgctgggcacattcctgagtgttcctgagcctctttcagtcccaggggtgaggtgctgggtgggtgcgctccctgggggctgggtggggctgaggatgtgagcgaacaaaccagacctagaaaaaaaaaagacagagatggaagcagagatgaaggcccagagagaggggttcacttgcccagggtcacacagctcaggaatctgaacctggctcaaaccagcattcacttctctcaaggcccacccaagggctgcagtgcagtggcacaatctcagctcactgcaacctctgccttccaggttaaagggattctcctgcctcagcctcccgagtagctgggattagaggtgcgtgccaccacacccggctaatttttgttattttaggagagacgaggtttccccatgttgttcaggctggtctcgaactcctgacgtcaagtgatccacctgcctcggcctcccaaagtgctgggattacaggcatgagccaccgcgcctggccaggaacagtctttactgacatgtgtttagggccatcgcacttcccccgctcttcacgggaattctctcttcactggctccactcaggtgccttttgtgagacagaaattcaagcgatgaggacagagatacaaacccgggatgggaggctgtgacatctgcccatagaaacacaattctgtttctaggaatttatccttcaactcgctagtacacgctcttcaagacatactgaaaaaacatgaaggattagaacagtgggtgtcgaatgctaccatttgtcttggaaatcaagcaaaacgaacgtgggtatggttgccgacgcatggaccatctccagtggaacaaggaactgccaacgttgattgcatttgggagggagatgaagggctgggatggaaagagctttattctcagtgcctcatcttttggttgttttggattttacaacatgtgcatgcattgcctacacaaacaaagacactaatttgaaaaggaaaaggggagaagaaaagagggaggaaagaaaaggggccggtgtggcagctcacacctgtcatcccagcaccttgggaggccaaggcggatggatcacctgagatcaggagtttgagaccagcctggccgacatatactgaaaccctgtctctactaaaaaatacaaaaattagctgggcatggtggcgcacgcctgtagtcccagctacttgggaagctgaggcaggagaattgcttgaatctgggaggtagaagttgcggtgagctgagatggcaccactgcactccagcctgggcaatagagcaagactccgtccctaaaaaaaaaaagaaaaaaaaaaagaaaagaaagaaaggaaagggagggaagagctcagtgtgggagaagattgggggtgggagtagagaattaatggaggaaaaagatgtgggggacaaaagcaaggtgtgagaggtgttggcaatggaaactgaatacctgttgagaagagccaggagggggcgacatggctttgaatctcttggggctgtgagattatgtgaaatgtgagaagactggacctcagtggggtgcctgacatatattcaggttacacctcccacaagcaggtggggctggacaatctctgtcaaccctccagccaggacccaagtgtctccctgtacattacttttggttgttgttgagacaagaatctcactctgctgcccaggctggagtgcagtggcatgatcttggctcactgcaatctctgcctcctgggttcaagtgattctcctacctcagcctcccaagtagctgagactataggcgcctgccaccacacctggcaaatttttgtatttttagtagagacaaggattcaccatgttggccaggctggtcttgaactcctgacctcaggtgatccacctgcttcagccacctaaagtgctggggttataggtgtgagccaccgtgcctcgccctccacatgcattttgagagccagctctctgggttgctttgtcccaactcacccagccttctttgaggcttccccaaaccctgggagcatttgacattgcagactgagctggacaatcaagggagaggtcttgctgacctgtcggggctggtggagtcatcagagattgtctttatcctcgccttcttcttctccacctcctcaacttcccctcttcttctggggcaatcctggagggcttcttgtaggaggcctctcccaccatctgcccagctctttctttctaatcttgttcaattgtgggtaaaggcacagctagttgggagcttggggagtttgatggggacaggggatgcagggagagggaggggcagggaatgctctcagggtccaaatttccttccattctagggttgcctgtgctcatctgtctgatggcttctctctctccctctcatccctgcctcctctttcctacctctcctcctccccttgtctctgtctctgacattgcttctatctttcttttttaacatctgtctcttttgctttttatctttgcctgtctctcctctgtgtctctctccttatgtctccatcttggtgtgtctctgaatctctccgtatctctgcctcctcctccccacatctctccccacctcccttccctatctctgtctctctccacagtcaaaaaagccaagtttgatggtgcccaaggtaagtggcttctcttgctgtcctgtcccgtgttgtctgtctgtctgtctgtctcttggagagactgggagggggtcccagcccagacttagagttgggctttatgtcttctgagcatcaggagaggggtcagaagattaaatagggggaggggcagccattggcctggggtccctgcagtttgtatgggggtccccgggtctccccagccatccttggagagtctgtaaggggtggggcttgccctgagccagtggctcttcccagggtcctaagaggtagaatggggactcttagatggggctacaggtgggaaaaatgaggctcagagaggcgaaaggagtttctttcttttcttcttcttcttttttttttttttttgagacagagtctccttctgttgcccaggctggagtgcagtggcgtgatctcggctcactgcaacctctgcctctgggttcaagcaattctcctgcctcagcctcccaagtagctgggattacaggcgccctctaccacgcccggctaatttttttatttttagtagagatggggtttcgccatgttggccaggctggtctcaaacttctgaccttaaatgatccgcccgcctcggcctcccaaagtgctaggattgcaggcgtgagctaccgtgccctgcctttttattattattatttttatttaaaatttttattttttgaggctggagtgccgtggtgcaatcatgttttactacagcctcaacctcccaggctcaagcaatcctcccacctcagccttccaagtagctgggaccacaggcgtgcaccactgcacccagctaattatttttttgtattttttagtagagatagggttttgccaagttgcccaggctggtcttgaactgctgggctcaagcaatcctccccccttgacctcccaaagtgctggcattataggcgcgagccactgcacccagcctgcaaagggggtttcttacagtcaattttgttccccttttctgggacctcagtctttttagctgaaaaccaatggcatggtcatttcgaagcagctggatgaagtatcccaaaattccacgtctccccacactgaggcccctcccttgtgccctgagggtggggttgtgggttagggggtgagtggcccagaaattcctgaggggatcacagcagatggtctagagcttgggaaggggcattggagagtccctgagccccagcgctgacccctactctcactgccacagagaaattcaacacgtacgtgaccctgaaagtgcagaatgtcaagagcacgaccatcgcggtgcggggcagccagcccagctgggagcaggatttcatgttgtgagtctgcagcgtctggcatgacctcactccctggcatagcccagcacctcgctggcccctcagggcagagctggacactggggaatcccgggtgactcccctctgtcctccctcttcccctgtccaccttatcaccttcacctccaaaacacatccccaatccaatcacttcccttggtctccgcagccaccagccaagtccctgctgcccgccagcctccgccctggcctcctgtgccatccacctcctacaatctgttctcctccaggcagccagggggatatttttaaaactgcaaatcacatcctgtcgctcccatgttccaaacttgtccgtggctcatcgacctcacaataaaacccacagccttccccgtgattcccaccaggttctcgctctatctccctcacctgccccagccacacccacctctgcccctttctccaacacttcccagcttgttcttgccccagggcccttgcacttgctctcctgtccccagctggctcttctccttcaggtctcagcttgtgtcacctcctcagtgaagccctccctgaccactacctccaagctacaatttcagggcatcatgccttcccaggccttgccaccaattttgtttgtttgtttgtttgttttgaggcagagcctcactctgttgcccaggctggagtacagtggcataatctcggctcacttcaacctccacctcccaggttcaagtgatcctcctgcctcagcctcccaactagctgggattacaagcgtgtgccaccacacccggctaatttttgtgttttaagtagagatggtgtttcactatggtggccaggctagtctcaaactcctggactcaggtgatccgcccacctcagcctcccaaggtgctgggattatagatgtgagccaccatgcctggccaatggaatttttttccttttttttttgttttgttttgagacagagcctcactctgtggcccaggctggagtgtagtggtgcaatctcggctcactgcaacctctgcctcctgggttcaagctattctcctgccttagcctcccgagtagctgggactacatgtgcgtgccaccatgcccggctaatttttgtgtatttagtaaagaccgggtttcaccatgttggccaggctggtcttgaacttttaacctcaggtgatccacccgcctcggcctcccaaagtgctgggattataggcatgagccaggagccaccaatcccagccaacagatatatatatcagatatatatatacttttttttttttttttgagactgagttttgcttttgttgcccaggctggagtgcaatggcgtgatctcggctcaccgcaacctccgcctcctgggttcaagcaattctcctgcctcagcctcccgagtagctggaattacagacatgcaccaccacgcccggctaattttgtatttttagtggagatggggtttctccatgttggtcaggctggtcttaaattcccaacctcaggtgatccgcccatctcggcctcccaaagtgctgggattacaggcgtgagccaccgtggttggccatattttaaaaatatatttcttaagataaatctttggtaaactgtaaattcagtgaatctcacatttcaccaagtttcctgctcccccgaattcacatatgtctagggcgaggcctgcagggaaagtgctttgaggtgggatcgtgtcctcacatcattagatggggatgacactgggaaggacagaggtgacccgaatgtccttatcctgtaggtaaagggagctatggagggtatttgagcctcttcctcacttacccagcatggtcagtgttggagggcccaaggaagggctcagaaagatgaaggtgcaggcgatgtgtaacatctgtccccacgccatccttcccggctttgaatcctggttcttttgtgataccccgagctcttaacttcccccactggcctcagtttcccctctataaaatgagaaccaccatgttaccctaccacctaggacactcccaggcctaggtttgaattgcagtatgctgtgtgttctcaggcgaatcaactaaactccctgggcccccgtttcctccctcctgctccctcgacgccccctctcctttttctgcagcgagattaaccgtctggatttgggactgacggtggaggtgtggaataagggtctcatctgggacacaatggtgggcactgtgtggatcccactgaggaccatccgccagtccaatgaggtgagtggcgggtggcctgagggtgaagaaggaggagtgccttgcagaagcccagagaccagtggactaaacaagatgggcatttatctcctatcccctgacaacatcaatgtagacacttgggattgccacgaaccttccaaagtcactagagcctatacaccttccatcttgctctgcctctctctccatgtggcttccaccttgtggccccaaatggctgctttaggctcaccattgcatcttcattccaaccaccgaaaggtgctggtggtgggggaggggggtggttcatgcacatctcattggccagaacttagacatgtggccaacaccttgctgcaagggattctgggaaatgtagtcctaggctggaaaatgtagtcctgggctgtgcttccctaggcacagctaaaaatgtattaccttggaaggagaaaagggataatataggcaattagcattttgtgtcccagatgggcagcagcaggaagcttacaccaactcataagaaggtgctaggtaacaaccctggcccctcagcctgtgcatcattgtcagcaggtagtagtgatgtctattttttatttaattatttatttttgagacagggtctcactctgttgcccaggctggagtgcattggcatgatcatggcccactgcagcctcgacttcccaggctcaagtgatcctcccacctcagcctcctgagtagctgggactgcaggtgcatgccaccaggccccccttattttttttgtatttttaatagagacagggttttgccatgttgcccaggctggtttcgaactcctgggctcaagcaatccacccacctcagcctcccaaagtgctggtattacaggtgcgagccaccatgccaggccgtggtcatgtctattcatctgtccccacagcccaatccctggcctcctaacctcaagtctcaacctctccatgctgaccccaggaaggccttattagctggagctctgatctgtcctcccctgtccatctgccttgggaggcccttcagatggcacctgcccactagcctcatctccccccacactctaacctctacccctcccagattccaccctctgaacctttgcatagtgtgttccctgagcttcaagtgtcctcctttccatcaccttctaaattcccatgtgcccacctccaggaagccctctctgacttctctctagatcctttagccctccttcctccctctgggctatgacctcatggggctgggggtgcctgtgtccagccctgtttccaagggctatgggctctcaggagccagggtgatgatggctgggcctcttggggaccctggcatcacccaatatgaaccaggcatagaggagggactggggactgttttttgtttgttcgtttttgtcattttattttattttattttattttattttattttattttattttattttattttactgtgagatggagtctcactctgtcacccaggctggagtgcaatggcacaatcttggctcactgcaacctctgcctccctggcttaagtgattctcctgcctcaacctcctgagtagctgggattacaggggcctgccaccacacccaactaatttttgcatttttagtagagatggggtttcgccatgttggccaggctggtcttgaactcctgacctcaagtgatccgcccaccttggcctcgcaaagtgctgggattacaggtgtgagtcaccgcacctggccagtgagtgtttgtttttgaatgaatgaatgcagagggacgaaatggttgcaacaatctaggtaagcgagtctaaaaggaagccttggctgggcatggtggctcacacctgtaatcccagcactttgggaggctgaggcaggtggattgcctgagctcagcagaccagcctgggcaacatggtgaaaccccgtctctactcaaaaatacaaaaaattagccaggagtgctggcacgtacctgtaatcccagcagctcaggaggctgagttaggagaatcacttgagcctgggaagcggaggttgcagtgagctgagatcacgccactgcactccagcctgggtgatagagagactctgtctcaaaaaaattaaattaggccgggcatggtggctcatgcctgtaatcccagcactttgggaggccgagatgggtggatcacgaggtcaggagattgagaccatcctggctaacacggtgaaaccccgtctctactaaatatacaaaaaatcagccaggcgtggtggcgggtgcctgtagtcccagctactcgggaagctgaggcaggagaatggcgtgaacccaggaggcagagcttgcagtgagccgagatcgcgccactgcactccagcctgggcgacagagcgagactctgtctcaaaaaaaaaaaaaaaagaaaagaaaagaaaaaagagagagaaaggaaggaaggaaaagaaaggaataaaggaaggaagggaggaagggaaagaaagaaaggaaggaagcagggagggagggagggaaggaaggaagggaaggaaggaaggacggaaggaaagatagagaggggacatgactttggttttggggtaggggtgagggggatgtctcatgcgagagtaggggccctggggactagtgaccagcagattcctgacccacacacctcctcccccaacccccaggagggccctggagagtggctgacgctggactcccaggtcatcatggcagacagtgagatctgtggcaccaaggaccccaccttccaccgcatcctcctggacacgcgctttgagctacccttaggtgagtacaggggcccgggaccttcacccagccccagccccagttgtggactcactcagctattccaggagagtgaacaggcctgggtaggtctagctgacccttcaggctgagaatgagggtccttcgccccggaggagagagagtttcttgggttgcagagaacagggatgtgaacctgctgggtgcgggccatgacctgggccccgtagcttcccagtgagctgcaagtgcttcacgaccgagggcgttcagggttgagctatcctggctgttccttggtgatgcctgagactgcaacagtgaactggggataggaggagagacagacagagacagagagacaaagagagacagagagagacatgtagacagaggctgagaacagatagtcccctcttcctttcctggaagcaccttgagtctctccacctcattcttctaagaacccatggctggccaggcatagcttctcatgtctgtaatcccagcactttgggaggccaaggggggaggatcatttgaggccaggagtttgagaccagcctgggccatctagtgagatcccatctctatgaaaaagttaaaaattcaccaggcatggtgctgggtttctgtagtcccaactatttgggaggctgaggcaagaggatggctcaagcccaggagtttgaggcttcagtgagttgtgattgtgccactgcactccagcctgggtgacagagcgagaccctgtctctaaaaaaataaaaataaaaacaatgggctgggcgtggtggctcatgcctataatcccagcactttgagaggccgaggcagatggatcacctgatgtcaggagtttgagagcagcctggccaacatggagaaaccctgtctctactcaaaatacaaaaaaattaactgggcatggtggtgcacacctgtaatcccagctactcgggaggctgaggcaggagaatcgcttgaacccaggaggtggaggttgcagtgaactgagattgtaccacttcactccagcctgggcaacagagtgagactccgtctcaaaaaacaatttaaaaattaaaaaattaaataaaaacaatggcttccccaccctcgtaaaggctaggaaccatctcctgtcatcctccaactattctccattcagctctgggaagaggggcgtcagaggcaccacggtacctccccttcatctccatccaccctcctgagaccgcagaccaacccacccctacttctgaggctttgccaaagaccccagaagccagggcccgggtggagctcagtaggcctggaggatggaggctgagcggcagggagggtcttccaggctcacagacagggctctgcttgcagacattcctgaagaggaggctcgctactgggccaagaagctggagcagctcaatgctatgcgggaccaggatgaagtgagtgttggggtgggaggggactgtttcttccttgtttctgtctctgtctccccatgattttcacttacagatgctgtttctctctaagcttgtctttccctgcatctctgtttctaactctttgtaacctgactgcatccatgacccatgtggtccagtctctagacaatgatggggtcaagaaacacacagaggccaggcgcagtggctcacgcctgtaatcccagcactttgagaggcgaaggtgggcagatcacctgaggtcaggagttcaagaccagcctggtcaacacagtgaaaccccgtctctactaaaaaaaaaaaaaaaaaaaaaaaattagccaggcttagtggctcacacctgtaatcccagcaatttgggaggctgtggcgggcagattatttgaggccaggagttcgagaccagcctggccaacacggtgaaaccccgtctctactaaaagtacaaaaaattagctaggcatggtggcgggcgcctgtcattccagctccttgggaggctgaggtgggagaatcacttgaatctgggaggcagaagttgcagtgagccgagatcacgctgctgcactccagcctgggcaacagagcaaggctctgtctcaaaataataatattgataataataataaaataaaaaattttacttttcatttggcggatacatgtgcaggtttgttacacgaatatattgcatgatgctaaggtctggaattccatggcattactcaaattgtaaacacagtacctaacgggtagttttttcactcttgtcctgctccctccttcctcccttctggagtccccaatgtctactgttcccatctttttttttttttcttttttgagacggagtctcgctgtgtcaccaggctggagtgcagtggcacgatcttggctcactgcaacctccacctcccgggttcaagtgattctcctgcctcggcctcccgggtaactgggactacaggcatgcgccaccatacctggctaatttttgtatttttagtagagacggggtttcaccgtttagctaggatggtctcgatctcttgacctcgtgatctgcccgcttcggcctcccaaagtgctgggattacaggtgtgagtcactgcacctggcccctagtcttttttctatctctgtttcttactttttctgtctctgtctctgtctctgtctttctctctctctctctctctctctctctctcgtgtgtgtgtgtgtctttctctccttttctctcttactccctgtatccttctcatagagagggagagaatttgggaggtagagggaagtcgggatgggacagaacagaggactatcctggtcccctggcctttggcccctctgagagaagccctttctccttcctccaccccttcagtattcgttccaagatgagcaagacaagcctctgcctgtccccagcaaccagtgctgtaagtacctcttgattcttccttgggtatcagctgaaatcctcatccctcccctaccccatgactgtcctgtctaaaggctcctccagtccggagatacaagtcctgagagtgcagtgtccacttacagtgtattagaaatcagcgttctgaggctgggcatggtggctcacccctgtaatcccagcactttgggaagccgaggtcaggagttcgagaccagcctggccaacagggcaaaaccccatctctactataaatacaaaaaaaatgaacaggcatggtggtgggtgcctgtaatcccagctactcagaaggctgaggcaggagaattgcttgaacccaggaggcagaatctgcagtgggccaagatcatgccactgtactccagcctgggtgacagagcaagactctgactcaaaaaaagaaggaaggaaagaaagagaaagaaagagagaaagagagagagagaaagaaagaaaggaaggaaggagaggagaggaaagaaagaaagaaaaagaaagaaaagaaaagagaaaagaaaagaaaggaagggaaggaaagaaaggaaaggaaggaaaggaaagagaagagttctgagacttgggcttgatgaaagacaagaaggaagaaaggaaggaaggaaggagagttctgagacttgggcttgattcccaccttgctatgtggtcatgggcaggccagtgtccctcttcgatatttaggggccctctctttgaaatagcgcatctgacagccccctcactctgagggctgttggaaggatctgggacatctagagacatgaaaaacacttcgtagatgcttcacttccattcatcagatcctggatgagggcgtataactcattccacaaactattcatggggccagcacaaaccccgagcagcccacgtcagatggtgcagacagacacaggcagaagatcctgccagcacatgtacaatgggcgcacgtacaacagacgaaggctgtttcactaacccattttcttagatggagaaactgaggcccaagaggagatgtgatttacccaaggccacacacccggcaactggcagagccaggatttgaacccaggtctgagggactccaactttgcttccctttccactgcaccttgtctgttaatgatgatgatgatcacgttcctcttcctatttccatcaaggctgcagttgtcaagattttttatcttcctgggccttgtggacactcagatgcatccagaccaggtcttttcatggatggaggagctgtctccctccttaaaatgacttcccattgctctgtggtggaggggaagtagcaaatcatatagcctcagggcagagctcccacctgctgacaccaaaaccccactgcactctttagcctccttctcctttcccatttactttcttttcttcttctctttttttttttcttggagatagagtctccctctgtcacccagactggactgcaatagtactatcttggctcactgcagcctctgcctcccggatttgagtgattcttgtgcctcagcctcttgagcagtggggattacaggcacatgccaccacgcccagctaatttttgtatttttagtagagatggggttttgccatgttggccaggctggtctcaaattttttacctcatgtgatctgcccacctcagcttcccaaagtgctgggattacaggtgtgagccaccatgcctggcaccttttccatttgcttttccatcttccttttctcccctctcacagctctccctgtatctgtgtctccctcaccatgtgtccaggagggtggtaatggaaccggggttgaggagggcttgggatactccagctggggtaccctgtttccacactaacttggggagtgatattgagaaagttccttggcttccctggccagggaggggtcatcactgcctggaaggaattacaaacccccgcttggtgaatggggtcattgagaggttgcaatgggggcggggccaggcagagtggaggcaggaaggcaatggcagagagcccctgggcagatggactagagctaggggcaggttcattttgggaagtggagaccaccactcagtatttacccacaagtcctcccttggagtattttgtcaccctatttctcagagaaagcagccaatggtcagaggagccaggattctggtccaggacatttttctgaagtgccaggtactgcacgtgataggcaaaggtggttctcgccctggccctgatgcgcagaggctctgtgactttgggctgctcactccatccctctgagccccatagtttcacccataaaatgagtagaatgataaaatccacccaacaggggtgtttcaaaccttagggtttccaatttagtgtgtctttccttatccaagagctgggagaggaaaagggtggagactgggagactgctaaccctagacatcatagtcagaaaaccctgggcccaaatacccactttgttacttcctgactgagcctttcctctcattgaaaactgagccaggcacagtggctcatgcctgtaaatcccagctactcggcctcacttgagaccacgtgtttgagaccagcctgaccaacatagcaagatccatctccacacaaaaaaacaaaaagaaaaccaccaagaaccaaaacattagctgaacatggtggtgtgtgcctgtaatctcagttactcaggaggctgaggtgggaggatcacttgagcttggaaattggaggctgcagtaagccgtgattgcaccactctactccaggctggatgacagagtgagaccgtgtttcaaaaaaaaaattaaagaaaagaaaaaaataaacaaacagggatatcccaccttcctcccaaggtggagagcagagacacagcaagtgtcagcaatgaaggtgatactcagggaaggtactcaggacaagagggacttttggggtggggggagcagttggggagagtgaggacagcaggggagcccccttcctacacccacccctctgtctgctcccttttattatttctgccccttctctttgtaggcaactggaattattttggctggggtgagcagcacagtaagtactcagcctgattgatgttctgggagttacatgggaaggtgggcaccttggtgatcccaaggcccctccccactaccacacagggaagcaaggagcctgtgacgagcacgtgggggccagagggctcagcaagacccctttcaagctttcctgtaccctgttcattagtgcctgtcttgggaggcctgccatctgctggggagtgggcaccgtcattggggtggggtggggggggttagcctggaggagtggacatttagaccaacaacctgcttgtctccagctgggcactgtgacccccgcaccccactccgccgtataacctcacctcctctcctgtagcccctgccttttggtcttcctatcttccttctccacttggaaatgtgtccagtgtcactcagagggtagtgggtgggcggggtctctaacagaacggcaggccagggacctcagggagatgcacacagggacctcagcaccccccaggtccacccacactcacacctcaccttccaccttcatttgctctcccagggaataaggcaagccaccccctccctaaattaaatgtccattttccttttcctttttttttttttttttgagacggagtctcactctatcacccaggctggagggcagtggcacgatctcagctcaccgcaagctctgcctccagggttcacgccattctcctgcctcagcctcccgagtagctgggactaaaggcgtccgccaacacgcccggctaattttttgtatttttagtagagacggggtttcaccgtgttagccaggagggtctcgatctcctgacctcgtgatccacctgccttggcctcccaaagtgctgggattacaggcatgagccatggcacctggccctaattaaatgtccgtttttctacctaacagcggttcccaacctttttgacacaagggaccagtttcatggaagacaatttttttatggactggggttgggggatggtttctggatgattcaagttcactgcatttgttgtgcactttatttttattattattattattacattatagtatgtaatgaaataatgatacaactcatcatcatgtagaagcagtgggagccctgaacttgttttcctgcaattagacagtcccatcttagggtgatgggggacagtgacagatcatcaggcattagattctcataaggagcacacaacctagatcccgcacatgcacagttcacagtagggttcacgctcccgtgagaatctttttttttttttgaaacagagtcttgctctgttgcccaggctggatggagtacagtggcgcaatctcgcctcactgcaacctctgcctccgagttcaagtgattctcgtacctgagcctcccaagtagctgggattacaggcgcgtgccaccatgcccagctaattttttgtatttttagtagagatggggtttcaccatgttggccaggctggttgtgaactcctgacttcaggtgatccacctgccttggcctcccaaagggctgggattacaagtttgagccactgcgcccggccttctatgagaatctaatgctgccgctgatctgacaggaggtggaactcaggcggtaatgtgagtgatggggagcaactgtaaatacagatgaaacttcactcgctcacccaaccgctgcccacctcctgctgtgcgccccagctcctaacaggccacagaccagatccatgccctaaagagttgctccctttctcccataccccgcctcttctagagaaatccagtcctctattctttctttccttctttcttttttcttttttttgagatggagtcttgctctgtcacccagactggagtatagtggtgcgatcactgctcactgcaacctccacctcccgggttcaagcagttctcttgcctcagcctcccgagtagttgggattacaggtgcacgccactatgcctggttaatttttgtatttttagtagagatggggtttcaccatgttggccaggctggtcttgaactcctgacctcaagcaatcctcccgccttggcctcccaaagtgctgggattataggtgtgagctaccatgcctggccagtcctctattttgaggataaatctgcctccttctctctccttcaactggggagtccttcactccttcctcatatcctggtggacaaacctaactccaggaccagtgaaactgttgacaaacactttggtccaccacaatttatcaaaaacaagttggttaaaaatcaacttaggccagccacactggctcacacctgtaattccaacactttgggaggctgaggcgggagggacgcttgagctccaaagtctgaaatcagcctgggcaacagagcaagactccatctctacaaaaaaaaaaaaaaagggccgggtgtggtggcacgcccctgtagtcccagctatttgggaggctgaggtgggagggttgtttgagcccaggaggttgggactgcagtgagccatgattgtgctactagactttagcctaggcaacagtgtgagaccctgtctctaaaaaataaatagattaaaataaataaataaaatcaattttgccaaaatgaatgcttggcagacagggcaagctcgataacaaccaacttgccacaaacgaacagtacggtcaaaagcaaagatagaccaggcgtggcagctcacaccataatcccagcactttgggaggccgaggtaggcagatcacttgaggtcaggagttcgagaccagcctggccaacgtgacgaaaccccatctctactaaaaatacaaaaattagccaggcatggtggtgcatgcctttaatcccagctactcgggaggctgaggcaggagaatcatttgaacctgggaggtggaggctgcagtgagccgagatcgtaccattgcactccagcctgggtgacagagcgagactctgtctcaaaaaaaaaaaaaacccaaaaaccaacaaacaaacaaaacaaaacaccaatgcagctaaagctgatagataattgggtcaaaaaggaaagacaattgaaccgaaaacagaccgttgggttaaaacaaaagtggttgaaaatagccagaagcaggcaatttagtcaagagctacttagtagaaagcagagcctgggcaaaagcaaaaagaatccaactcaaagtaatttacccttcataagcagataatttgtcaggaaaaaaaaaaactgggtaaacatgtgcacagcctcatgcaatttggctggaaacatctcagcccggtcaaggacaattggatagaaagaatgctggccggccgaggtggctcacggctaatcccagcactgtgggaagctgaggcgggaggatcgcttgagcccaggagttcaagatcagcctgggcaatatagggagatcccatctctacaaaataataaataaataaaattagctgggtgtggtggtgtgtgcctgtggtcccagctactgtggaggctgagatgcgaggatctcttgagcccaggaggtcgaggtttcagtgagttttgatcgcgccactgcgctccagcccaagtgagagagaccctgtctaaatgaatggatgaatacagcaaaagtctgaaggtttcactcattgctctgagattttggggtctgggggttttttcatttttttacatcctcttccccttacccgtacttctcatgttttcttcatggactactttttctttttttctggatacagggtctcattctgacacccagactgcagtgcagaggcgtaatcatagctcactgctacctcgacctcctgggctccagcaatgctcctgccataccctcccgaatagctgggactacaggcgcccaccaccacgcccagctaatttttaaatttttttaaagagatggggccttactatgttgcccaggctgatcttgaactcctgggctcaagtgatcctcctgccttggcctcccaaagtgctgggattacaggcatgagccactttgcctggcccatggactaggttttactttacttttattttattttattttttggagacagggtcttgtgtcacccagcccctggagtgcagaggtgtgatcttggctcacagcaatctccacctcctgggttccagagattctcctgcctcagcctcccaaggagctgggattacaggcacatgccaccacacctggctaatttttgtatttttagtagagacagggtcttggtatgttgcccaggctggtcttgaactcctggcctcaagtaatctgcccgcctccgcctcccaaagtgctgggattacaggtgtgagccactgtgcccggcctttgctttacttttaaaataaagataactttatcattctttttagttaaaaaattaaaaagattgcattattgaaatcttttttttcagaaggtacagaaaagaataaagaagaaaatgtacaatccccaattatgccaccacccagacatgttgcttttagaattggaatttttttttttaggatttagaaaacaaactgttcttttttctaaggaaaccaaactaaaccaaaatatcttttagttcttattaaacatgaagtttggcaactctaccacattccattttagcaatagattccaagtgtgtggtggctttgccaagctaagaatctaatcctttttgaaaaagacagaaccatgtctcagcatcttcttggttcttgcatatttgttaagttcaggtctggggtcgtcaccctcagatatcaggaaatctctaagggttaaagagcgttccgtccactttgaactcctgatcttcttccaacttccccctgactgtctgtgtgactcctgttatgggaaactaactgctcagtggaaaaaaatcccatgtcattttcagagatcctgaccttcagcaaagttgttcttcacatttgagctgaaggatgccctctactctgtccctgttttcttcacattgtaggtcctaccctagcccctagactttaggtccaatttgacccacataaagcaaagcagggttttcacctcccttgttctgaagcctatatctctattaacatgacccacaattctttatccatactagtaaaaaattctcttcttcggtagcggcaccgtaagtctgggaaattgtattggctcccatcatgtgcactctctcttcctcaccctcttcccataccgcctctctcttccagacgatgaccccgacagtgcagtggatgatcgtgacagtgactaccgcagtgaaacgagcaacagcatcccgccgccctattatactacgtcacaacccaacgcctcagtccaccaatattctgttcgcccaccacccctgggctcccgggagtcctacagtgactccatgcacagttacgaggagttctctgagccacaagccctcaggtaggtgctgcggagtgcagcatgcatgtgggtcctgctgtgtctcctggagtggagagaggggtacggggctggacagagctggaggtggagttccagaagggcagaattcacccatccaggagggcagaattcacattttcttgtgggaattaaccatttttttctttttttttttcttttcttttttttttagacagagtcttgctctgtcacccaggctggagtgcagtggcaccatttcggctcactgcaacctccaccacccagactcaagcgattctcctgcctcagcctccccagtagctgggactacaggcacaggccgccacaccaagctaatttatttttattttttgtggagatgggggtcttgctccattgcctgggctagtcttgaacccctttttattcctatagcatcctcgaggctggccagggaagagggtctcaggcgctaagattggaggaggtatctaatcgggaaatgataaggttcgttgaatgactaagtgaatggttagaagagggctgagtggtggcccctatgtccccctgcctgtatttagacagagacagatattgctgttgacgttgagcacgatcctgggttcacataagcagcacataacagagggcctgacctggccagacagtggggggcatttgcacaagtaagtgatgtttcagtgggtaccagagggtgggtaggagtttgctagataaagcagacaaggaagagaccacggcaagtgcacaggtttatggtgttggagagacaacgaggtggccagcgtggccggaatggaggaggcggggagaagtggagggaggtatgcagggggagtttggagcgtatgggccacacagggctagcacagattttattccaaggcactagggagccatggtaggatttagagcaggggagggaggtgattggatttacaattccctgtggcttccaagggaagcaggaactgtggagacagcatctggtagagacagcagaggctggcctgggttggggcccggggtacggggaagtggatgcatttcccatactttttccaggtggaggacaggagacctgctgcggggctgtggtgatggcgctgaccctgagttcccagcacagtcaattgcctgttccccacagaccgcaaatgccacaaaagtgggaatttttttttttttttttttttttttgagacggaatttcgctcttgttgcccaggctggagtgcaatggcatgatcccagctcactgcaacctccacctcccgggttcaagtgattcttctacctcagcctcccgagtagctgggattacaggcatacgccaccatgcgcagctaatttttgtatttttggtagagacagggtttcaccatgttggccaggctggtcttgaactcctgacctcaggtgatctgcccacctcagcctcccaaagtgctgggattacaggcgtgagccaccgcacctggccaaaagcggggatttttgtcttttctgatcatggctgtgttttccagcccctagagcaggacctggcacatcgtaggtgctcagtcggtgatgagtcaatgcctgtgtgactggggctcaggtgggcagtgactcggtaccccagtccctgaaccctgttccatccccacagccccacgggtagcagccgctatgcctcttccggggagctgagccagggaagctctcagctgagcgaggacttcgaccctgacgagcacagcctgcagggctccgacatggaggatgagcgggaccgggactcctaccactcctgccacagctcggtcagctaccacaaagactcgcctcgctgggaccaggatgaggaagagctggaggaggacctggaggacttcctggaggaggaggagctgcctgaagatgaggaggagctggaggaggaggaggaggaggtgcctgacgatttgggcagctatgcccagcgtgaagacgtagctgtggctgagcccaaagacttcaaacgcatcagcctcccgccagctgccccagggaaggaggacaaggccccagtggcacccaccgaggcccccgacatggccaaggtggcccccaagccagccacgcccgacaaggtgcctgcagctgagcagatccctgaggctgagccacccaaggacgaggagaggtaacaggacggggccaaggggccagaggggctgccacaaggtcacagggtcagcagggctatgatggggtcagcgtggacctgggaagttgctggggctgaagttgtcacaggctcttgggtttcttaaagggatcagaggtgacagggcagagtagggaggtcaaagagggttcaaggggccgtgaaggagacttagagtttgtgatcttagtggttgctggggtcggagaggtcaagatcctgggcaaatggggggccagaggggctgggctggtgaaaaggaagccgaggaacagtgaagtgtcacagaagcattgggtgtgtgggaaggggtgaggggcactgaggtgggccaggggactcacgtgggtcctaatgtctctgaggggtcagagataccatggggagctatgcatcacttgcacagctggagggtcaagggaagttcaggggtcacggagagccagcccagagcctcatagcgttccctgcctcagtttcaggccgagagaggatgaggaaggccaggaggggcaggactccatgtccagggccaaggccaactggctgcgtgccttcaacaaggtgcggatgcagctgcaggaggtgagtgacacgcccagccatgcccctgccacacttagccaggccacgcccaatggcaggcccacacatgttttggccacacccacacctatattggccacgcccaacaccccacccgtacccaagcctagcctgggcatgcccagtgcagtgcccacactgtgcccatgccccagccactcgagcccttccctggccccaccctcattcagcctggcctcacccaatgtcaccgatgccccacccactctgacccttccttagccacatacacacacagcccagggtatccccatggcccgtccacaccagcctggctatgctaactcccaaaccatgcaccaagtctggccccacacatccccagcccagcctccttcccaatgtcagcctggggtttctagatctagagggaggagagaaaactagggctgaaactgggctgaagtcatggttagggatcaaggtcagggttagttcagccatcaaagatcagaggtcagggttagaattggtgttaggcaaaggggtcagtgcttagggccaaattggaggtcattgtcagggttagggaaaggtctggggccggctcagtaggggagtaggaatcaagggaagcaatcagcatcaggggcaagctgactgtttagatgggcagcagggttgaggccattcgtttattccttcattcagctcaccctgggctggggacagctgtgttgctcttcagagttaccctccaaactgcagtgggattggcaggaggagattctaaacagactcccaaaggtttagaccatcaaatatcagagccacaaaattaccaaacgaaatttcttaacagttttaaaatagaaatctattttttgactcagcagtacatacatatggttctaaatgagacaggtggaagaggacacagtgaaaaggctgttggataacttccactaggtcaagaaattaaattgaaaaaatagcagctactattggaactatgtgatatacaagaaaaacaaactatggaactagtaatatgacaagaaaaagacacatggaaggctctctgggtttcataggaaaagcataaaactaatccaaggcaatcaggaaaactccctggaggagatgacacttaaggagaagtgatggccgggcgtggttgctcaagcctgtaatcccagcactttgggaggccgaggtgggtggatcacgaggtcaggagatcgagaccatcctggctaacatggtgaaaccccgtctctagtgaaagtacaaaaaaattagccggctgtggttgcaggcgcctgtagtcccagctactcgggaggctgaggcaggagaatggcgtgaacccgggaagcggagctggcagtgagccgagatcgcgccactgcacaccagcctgggtgacagagcgagactccatttcaaaaaaaaaaaaaaaaaaaaaaagagaagtgatatccaaaatatttatttcaacaatatttcaggagctcatgcctgtaatcccagcaatttgggaggtcgaggcgggaggctcacttgcactcaggagtttgagaccagcctgggcaacgtagtgagacctcatctctaaaagaaaaaaataaaaaatatttcagagccggatgcagtggctcacgcctgtaatcccagcactttgggaggcagaggtgggtggatcacctgaggtcaggagttcgagaccagcctggccaacatagtgaaaccccacctctactaaaaatacaaaaaattaattgggtgtggtggtgggcacctgtaatcccagctactaggaaggctgaggcaggagaatcgcttgaacccaggaggtggaggttgcagtgagctgagattgcgccactgcactccagcctgggcaacaaaagggaaactccgtctcaaacaaacaaacaaacaaaatatatatatatatatttatttcaggagctgagtgtggtggcttgtgtgtgtggtcccagctactcaggaggctgaagcaggaagatcacatgaggccaggagttcaagaccagtctgggcaatatagcaagaccctgtctttaaaaaaaaaccaaacaccccctcaatatttcagaacagaagagtgctgcctgtgttcctcatagggccacccctccccccatgttaaccctttagttacaagatggagggaaggtaggggctgtggaccccacgtctatgctgagacccgaatgataaatagaggctggttggctgcagaaggtgggaagaggtacaagctgaagtcacaactcctggaaagagcccgaggcaagagagcagatgaacaagtgagactatagggggtttagggaataatcaatgaagagactgaggtccattgaggtggttgaaagagaagagcttctttctcccccagcccatcttacagatggacaaactaaggcccagagatagccacggagcttcctaaaatcactcagccaggactaggacccaggccatctgactcccagcccagaggggagtcagaagtgggtgtggagcctgctctggggaggggagggacagccacactgaccatatctgtctgtctgtcctgcaggcccggggagaaggagagatgtctaaatccctatggttcaaaggcgggtaagtaatgaaactgtgagcggtgggaggaagatttgaaccccaagtccaaggctggtttgcgcctgagcctcagcccagagaggaggaggaggggttccatttagacaggggacagattgagcttgggcaaaatacgtcactttcataaggcacacagccatacgcagacacaaaagacagtaatctttgaagacagggcggggtgcggtggctcatgcctgtaatcctagcactttgggaggccgaggcgggaggatcatttgaggtcaggagttcgaaaccagcctggccaacatggtgaaaccctgtctctactaaaaacacaaaaattagctgggcatgctggcaggcgcctgtaatcccagctacttgggatgctgaggcaagataattacttgaacccgagaggcagaggttgtagtgagctgagattgcaccactgcactccagcctgggagacagagcgggacactgtctcaaaaataaacaaacaaataaataaataaataaatgtaagataaagcagacactatgaggaactagacactattgggaaatgtacactatatatttgtaatcctggagcaaaatttttgcgttgattctatttttaaatctctttgattattgctacagttgtgggctctttaattaaagatagttgtttagtcttcagtggagaatgtggtcatgtctaacctcttatgttatggattttcaggttagaggatgcacaagatattttaaaaatatgctcaggttttaaaatagcagcacgatagtgaccttccttgtacgtccttggcaaacaggcagggagacagcagctgggactcctaaataaatgagcaggtaccctagctagagagccttggatgcgggtcctgaaatgcggattcctgtgcacgtgatttatgaaggatgtgctctcgggagaaacctgtcagacaaggggaggctgccgagaaaagatgtggtttaggagtctctaagcctcagttggctcctgcagaaagctttgtaggttgaatagccctgcagggcttgtctggagtcattggctgcaggcacctatctcactggccagtgaaaaccttttcggggccaggcttggtggctcacgcctgtaatcccaacactttgggaggctgaagcaggtggatcgcctgacgtcaggagttcaagaccagcctggccaacatggcaaaatcccgtctctactaaaatatacaaaaaattagccaggtgtggtggtgcatgcttgtaatcccagctatttgggaggctgaggcaggagaatcacttgaggctaggagttcaagaccagcctgggcaacatagcgagacccccatctctacaaaagaatttttttttaattagctatgtgtgggccgggtgcagtggctcatgcctgtaatcccagcactttgggaggcctaggcggttgatcacctgaggtcaggagtctgagaccagcctggtcaacatggtgaaaccccatatctacgaaaaatacagaaaattagccaggtgtcgtggtgcgcacctgtaatcccagctacttgggaggctgagataggagaattgcttgaacccaggaggcagaggttgcagtgagccaagatcataccattgcattccagcctgggcgacagagcgagactccatctcaaaaaaaaaaaaaaaaaaaaaatttgctgggtgtggcggtgtacacctatggtcccagctactcaggaggctaaggtgggaggattgctgtagcccaggaggtggaggctacagtgagccgtgattgtgccactgcactccagcctgggtgacagagtgagatcttgtttcgaaagatggctgggcatggtggctcacgcctgtaatctcagcactttgggaggccaaggtgggcggatcgcctgaggtcaggagttcgagaccagcctggccaacatggtgaaagcttgtttctactaaaaatacaaaaattagccaggcgttgtggtgggcacctgtaatcccagctacttggggagctgaggcaggagaattgcttaaacctgggaggcagaggttgcaatgagccaagaaaggtttcatctatattttattttatttttgatacagagtctcgctctgtcacccaggctggattgcagtggcgtgatctcgactcactgcaacctctgcctcctggttcaagcaattatcctgcttcaacctcctgagtagctgggactacaggcatgcactaccatgcctggctaatttttgtatttttagtagagacggggtttcaccatgttggtcaggctggtctcgaactcctaacctcaggtgatccgcccgcctcggcctcccaaagtgttgggattattacaggcgtaagccaccactcctggcctcatttatattttagtatattatttagatttgccttaattcacgttttcattcaaggtgggcaattacatcatctccccatttttcagatgaggagggtgaggccgggtgtctccatttcttaggactgctgtcacaaagtaccacagacgggtagcttaaaaccacagcaatgtattctctcacagttctggaggccagaagtctgaaatcaaggcattggtagagccatgctccctctgaaatctgtagaagagaattcctcccctgcctcttctagcttctagtttgcttgaattccttggcccccgagtattggtttgtagatacgtcgctttctgcctctgctgtcacagagccaccttctccccatgtatctcttcttctaaggacaccagtcatattagattaagtgtctaccctactccagtatgaccttatcttaactaattacatctgtgacaaccccgtttccaagtaaggtcaccttctgaggtagtgggggttagggcatcagcgtatcttttttttgagggacccaattcaaccccttaacacctgggggaggttgaatgttctccccagggctctatgtgaggaatagggacatctgggccctcagtgtccctctgtgtctttcagcccagggggcggtctcatcatcatcgacagcatgccagacatccgcaagaggaaacctatcccactcgtgagcgacttggtaagcaccctcgccctttactccctgagcagagcaggcctgctataagcagtgcacaacctgtggaactgcccatggtcaacctggggttgggggccagctaggcattctctgagactaatgtagtctgatttctacccccatgtgctgcgtggacacttcaggccatggtgagtgatggcgacatgggcctcttgggatcttctccccaaactcccccactgggatcttctaacaggcccccaattcatggattatgggggaaccttgccatgtcccactgtgttggatgctgtgagtcaggggtgcggggctgctcactttccagggtcccttctaactcaaagcctctgtcacctcctaattctttgttgggtctggcccggatgctgtcggtgacctgtttccaacttgcggttgtggctccaaaatgtggctgctggccccatccgtggctgtgaactctcctggtggtgatctcatggatgtcaacccccccgactccctcttctgtgacctccccagtccctggtccagtccaggaaagcgggcatcacctcggccttggcctccagcacgttgaacaacgaggagctggtgcgtggggcacctctttttccccgggtcagggatggactgggctaggagcttctggaattgtccaagcctctgtgggtccccgggaagaggaagggtgtgtgtgtgacacagactggcagaaggcagcgtggattccgacagcgtggaggcaggggagggcctgggactttaccgcgctcagtgatggggcggaatggggcaggacagggacagcgccaggttaggcgcccccaaaccgcaccccatccctccctgcagaaaaaccacgtttacaagaagaccctgcaagccttaatctaccccatctcgtgcacgacgccacacaacttcgaagtgtggacggccaccacgcccacctactgctacgagtgcgaggggctgctgtggggcatcgcgaggcagggcatgcgctgcaccgagtgcggtgtcaagtgccacgagaagtgccaggacctgctcaacgccgactgcctgcagcgtgagcgcagggcctgagcgcagggccacgggttggcgtggggagccccggggcttgcatggcggggaaaggctgaagggcaaggcgatgggtggggcaccatggggcggggcaaggggcggggcgtcgtgaggcaggggagggactcgctgaaagggaggggtggggttaaaggcgcaggactccctggtggaagggctagggggtcgaggggtggggaaagaagctgtgagggtgtcagagaggggtggggcagggctcagggactagctaggttgtgcaaaggacaggcttagtgagaagaggcagctcccctggataaaatgggttagctaggggtagctctgagaggaagaggggctcagagagggaaggggttcagactgcgagaggactgtggagggggggattggaagggggaggggactcagaggaggtgggaccaagagaaagggaggggtctgagagatggaaggggctcaggaatgtggaggggactcgcaggggtggagatcagagacgaggaggggattcagaggagtggggctcagagaggcagcgggactcagaggggtgggactcagagagggggcggagattcagaggggtgggacgcagagggggcggggactcagaggggtggggcttagggaagaggccttagaggggtggggctccaaaaaccagtggggactccaaggggtgcggggcagagaagggcaggacgcagggacatggcgactcagaggagcatggctccgagaggaggtggaagggtgaagggtctgggtggggggtacagaaagcaggtaggaaaccggcttcagggggtgacacctgagtccccccggttgatgagtaggggcggggctctcgccttggtgaggggccactatgcagggcgcgctggggtcagcgccggcctcgggcgtcctcaggggctgcggagaagagctccaagcacggggcggaggaccggacacagaacatcatcatggtgctcaaggaccgcatgaagatccgggagcgcaacaagcccgagatcttcgagctcatccaggagatcttcgcggtgaccaagacggcgcacacgcagcagatgaaggcggtcaagcagagcgtgctggacggcacgtccaagtggtccgccaagatcagcatcaccggtgagggggcgtgggccgccatagggcccctccttctccaccctctgagacctgtctcatgccctgtcccatggcctggtccctctcctttgcagcgcgcactcactcggtcgggtgcattccatctcatctccgtgtgtgcacgcatatggggggacatgcgtgcctccaaacgcctgttcatggcactgtatctgggtgcgtcttggatggtgccctccagaatgagactccaagaagaggaattgagggtgcatggtttatctggaaagtgaccccagaaagtcctggtgaggggcaaggacttgagacagggatggaaggagtcctgaaggagatgccgttgagcagacaatgctgtgggcaagggggtcagcccatcctggggccactggggacaatgtcgagcatgcagctcagtgtggccccacttaggggcgaggatactggcttttctccacacccagcctcatgtgggtttccccataaacagacctgaggatgaggatttcaggttatgtgggttatccaggaagtgaccccaggacggagctggggtggtaagtcagggagggaaggaaccctgcagggggcaccaacgagcaggagatctcggggggcaatgggttcttgtaagtgtctgacctcagggagttcacacctgagagaggtcctacccatgcgcaaggcagctggggcaccttcccatccttcatggtttggggactccttcccagcactttgggaggctgaggctgaggcaggcggatcacctgaggtcaggaattcgagaccgacctggccaacatggtgaaaccccatctctactaaaaatgcaaaacgattggccggatggggtggcacgtgcgggtaatcccagctacttgggaggctgaggcaggagaattgcttgaacccgggaggcggaggttgcactccagcctgggtgacgagcaaaactctgtctcaaaaacaaacaaacaaacaaacaaacacacaaaaaacctctctggcaagcccagcgtgccctgccattgctctccagccatgctgcaggtgtcccaattatcagcctgcagcgtgtggcagtgactgtccccagcatgcctcgcttcttgagtgcacacctgtgtatgccctctgtgtcttccagtggtctgcgcccagggcttgcaggcaaaggacaagacaggatccagtgacccctatgtcaccgtccaggtcgggaagaccaagaaacggacaaaaaccatctatgggaacctcaacccggtgtgggaggagaatttccacttgtaagtgcctggctgggtccctggccccatgtggggcatcctgctggggcagccaaaagagggctccaaggacaggactcacactggctgcctgacccctgcctctgcctcctcctcccgccacagtgaatgtcacaattcctccgaccgcatcaaggtgcgcgtctgggacgaggatgacgacatcaaatcccgcgtgaaacagaggttcaagagggaatctgacgatttcctggggcagacgatcattgaggtgcggacgctcagcggcgagatggacgtgtggtacaacctgggtgagagaagctgggttctgaagcgggtggggacgggtcccagggatccagccagagcatggagcccacgaagagcagagagatgtgttctcaggaggagtttggtcgaggagcataatctgatgggggaggcagggtctaggagcccagtttgatgggagcaggaagcacccaggccaggagggtgagcagagcccaggaacctcttagttcagcttaggagggagggtggggacagaatctccatgttctagtctaatgggggaggcaagaagcaagaactgggaatcctagtctgaggggagagacagagacctaggaactaaatctaagaaagaagattgagctttaagaatccaggctggggccaggtatggtggctcacgcctgtaatcccagcactttgggaagctgaggcaggcagatcacatgaggtcaggagttcgagaccagcctggctaacatggtgaaaccccgtctccactaaaaatacaaaaattagccaggcgtggtggtgggtgcctgtagtcccagctacttgggaggctgaggcaggagaatcgcttgaacccaggaagcggaggttgcagtgagctgagactgcgccactgcactccagcctaggcaacgagagcaaaactcccactcaaaaaaaaaaaaaaaagaatccaggctgggggcgccgggcagtggctcacatctgtaattaatcctatcactatgggaggctgaggcaggccaatcacttgagtccaggagttcaagaccagcctgggcaacatagtgaaaccccatctctataaaagatacaaaaattagctaggcagggtgacactcacctgtggtcccacctactctggaggctaaggtgggaggatcccctgagcctgagaggtaaaggctgcagtgagctgtgatcacactgctgcactccagcccagggaacagagtgagaccctttctcaaaaaacaaatatggccgggtgcagtgtctcacgcctataatcccaacagtttgggacggctgaggcaggcagatcacttgaggtcaggagtttgagaccagattgtccaacatggcgagatcaagcccctgcactccagcctgggcaacaaaggaagactccgtctcaaaaaaaaaaaaaaaaaacaaaagaaaaagaaaaaaaaaatggaatccaggctgggcatggtggctcccacctgttatcccaacatattggttgggtggctgagctgggagggtcacttgagcccaggagttcgagaccagcctgggcaacataacaaggcctttgtctccattaaaaaaaaaaaaatagctgggtgtggtgatgcgtgcctgtagtcccagccacttgggaggctgaggtggaagcatcatttgtacctgggaggttgaagctgcagtgagctatgatcatgccactgcactccagcctgggcgatagagcaagactctgtgtccccccgaccccccaccacaagaaaagaatccagtctaaagagggaggtattgtttgggaatcctaatctgagatggctgctacagatcaaagaaccctgtgtaaggaagttgagtagaatctggggagtccaatctaatggaggaagcagggtgtatgagcctaatctgagggggtagaaaggacccagcctgagagaggtgagcagagcccggaaacctcttagattggaggacatctagatatcccattctgatgggggaggtgggagacaagaccagggggtcctagtctgaggagagaggcacagacccaggaacagtgtctaagatgtgtagtctccggggagcatgatctgatgggggaggcagggcctgggagctcagactgggaaagaagtggggcctagggctgctagtctaaggagggaggcacatagccaggaacttaatctgagtgaagaagcaaggtctgggatccttgtttgaaggaagagacagggcttaaggaattctagcctaaacagggagactcaagcctagggagccaatcagaagagagacaccaaagccttagtttccggaagtctgggaggactacagaatcgtgtatctattaaaaattgccttgggaggccgaggcgggcggatcacctgaggtcaggagttcgagaccagcctggccaacatggtgaaacctcgtctctactaaaaataaaaaattaggtgggtgtgatggcacatgcctgtaatcccagctactcgggaggctgaggcaagacaattgcttgaactcaggaggtggagattgcagtgagccaagattgcgccaccgcactctaacctggaccacagatctcaaaaaaaaataaaataaaaaaaaatttgcccatggctggccgggcgcggtgactcacgcctgtaattccagcactttgggaggctgaggtaggtggatcacctgaggtaaggagttcgagaccagcctggccaacatggtgaaaccccgtctctactaaaagtataaaaattagctgggcacggtggcaggcacctgtaattccagctagttgggaggctgagacaggagaatcgcttgaacccaggaggtggaggttgcagtgagctgagatcgtgccactgcactccagcctgggtgacaagagcaagactccctcttaaaaaaaaaaaaattgcccactgctatagtgggaagttctgacactgtcttctttttctgccctgcagacaagcgaactgacaaatctgccgtgtcgggtgccatccggctccacatcagtgtggagatcaaaggcgaggagaaggtggccccgtaccatgtccagtacacctgtctgcatgaggtgagggtcattgctcggcccctcccatgccacttccactcaccattcctgcctgcccagctcttcctctttctggccacaccatccacactctcctggccctctgagactgcccgccatgccattccctttacctggaaaactcctccctatccatcaaagtccagattcagggtcacctcctctgggaagcccaccttggcctccaggttgactctcactactcatcatcaggttcttccttctattccagccctaaccactcaggattgggccgtttgtgtctgggtatgtctcttccagctgcctgggtttcctggaaagaactcttatccccaggaactagtttgttgaataaatgctggtgaatgaatgaatgattgaacagatgaatgagtgatgagtagataaaaggatggatggagagatgggtgagtacatggatggatagatggatgagttggtgggtagattcgtggctagatggatgatggatggatggacagatggatggatatatgattgaactattgaaagtatagatgtatggatgggtgaatttgggggtaattgttagatgatggatgagtatagatgaatgatggatggataacttgatgagtggatagatagattgctggatagatgattgactgggtggatagatgaaatgttggatgagcagattaagttgtattggatgggatggatggaagtgtggttgagttattagaaggaagattgagtagataggtgaatttgttgatagtcagatgggtagataggtagatggatggatggatggatggatgtataggcagatggacaaatggatgaatgggtgggtggatgaatggaaggatgtgtggttgaactattgcaagtattgataattgggttcataatttctgaatatttagatggatggttgtgagtggctggtggacagacgaaaaatggatggttggataaattgatgggtggatggatggttggttgtatgaaagaatgaatgattgggtaggtggattaagttgcggatcaatgtatgggatggatgaatggatggatggatggatgtgtggttgaattactgaaaggttggaagagtggatgggtgaaatttggggtagttagatgggtgggtgtgtggatggataaaagagtagatgaatgaattaatgaataaacaggcagatggatgatgtaagctgccccagaccctgggacctctgacccccggcgaccccttgcactctccatgacactttctctcccatggtggcagaacctgttccacttcgtgaccgacgtgcagaacaatggggtcgtgaagatcccagatgccaagggtgacgatgcctggaaggtttactacgatgagacagcccaggagattgtggacgagtttgccatgcgctacggcgtcgagtccatctaccaagccatgacgtgagactgcaggctggggtgcaggaaacaagagggaggtcaccaagtgggcactggcaggtctggggtgggagggattctcagggggaagacccagagaggaagtgggcgtggtggtggtgggttccgtaattcccctggagatcaggagagcgctgtgattgacacctggagtgacatggaagagagggaagacttagaagcagagatagaggctgggcacagtggctcaagcctgtaatcccagcattttaggaggctgaggtgggcggatcactccaggtccaggagtacaagaccagcctggccaacatggtgaaaccccatctctactaaaaatacaaaagttatccaggtgtggtggcttgggcctgtaatcccagctactcgggaagttagggtgggagaattgcttgaacctggggggcaggggttgcagtgagccgagattgcgccactaaactccagcctgggtgacagagtgagactccatctcaaaaaaaaaaaaaaaagaaagaaagaaaaaaagaagcagaattagagcaactcaaatggatataaagatggaggggagaggagatgcggagacccaaaggacagaatcaggcaggggatggtttaggaggtgacagagacccactcacagatgctatcttggaggcattctaggggggatcttggtccatcctggctggacccctggcctagtgcctcctgctccctctctccagccactttgcctgcctctcctccaagtatatgtgcccaggggtgcctgccgtcatgagcaccctgctcgccaacatcaatgcctactacgcacacaccaccgcctccaccaacgtgtctgcctccgaccgcttcgccgcctccaactttggggtcagtcctggggactgggattggagaattagggagagcccaactttatgccggtcagatctgatgtgacttctgggtccccagtctagacctgattgctgtgtgactttccaccagtcacttcccgtctctgagcctccatttattcctctgtagaatgctatagaatggaggtcgggcgcagtggctcatgcctgtaatcccagcactttgggaggccgaggctggagaatcacttgaggccaggagttcgagaccagcctggccaacacggtgaaaacccgtctcttctaaaaatccaaaagaaaattagctgggtgtggtggcctgtgcctgtagtcccagccacttgggaggctgaggcaggagaattgcttgagcctaggagtcggaggttgcagtgagctgagactgcgccactacactccagcctgggcaacagagcaagactctgtctcaaaacaaaaaaaaaaatgccatagaatggaaatggacattatgagggtggtggatgaggtatacttactgtacagtaggcgctcagccactatggcggccatgtcctgcctccatccatcctccctcctccccttcctcctcagaaagagcgcttcgtgaaactcctggaccagctgcataactccctgcggattgacctctccatgtaccgggtaggaagtgtgtgcatgagaatttgctcaccccacgcacatgtgtgtgcctggaggggaaagctaccagcaggtgtgtgcatgggaggacccatcttcgcatgtacgatgtgttcctccagcatgtgtgcacctgcatgggcactacctgccgtaatgtgcttggtgcatccaagcagatccccagttgcttcgaggtgctctaatcaacccccagcctgaaaccggggagcctttgtatccccctgaaaagcagtctcccatcctccccacgccttcttccataaccccacatctcttcctgctcccaacagaataacttcccagccagcagcccggagagactccaggacctcaaatccactgtggaccttctcaccagcatcaccttctttcggatgaaggtaggaaaggatccagttgcctttgctctccaaggagggttctctaggatccccagctcccaggacaagtgacattcctcctggagagtggcagcctttctcagtaaccttttcctttcataccacttcatttccttaaacccaaaatgactcgcagtcacttcctggggacctcctgtcacagctgtggccgcctatgcctctctccctcccttcaggtacaagaactccagagcccgccccgagccagccaggtggtaaaggactgtgtgaaagcctgccttaattctacctacgagtacatcttcaataactgccatgaactgtacagccgggagtaccagacagacccggtgagactccagatgggtcagaagggaacagtgatgccaactagacacaggcttcagtcccagccctgccccttcccgagggatttaactcttctgaacctcagtatcttcttctataaaatggggtcattaacccacatctcaggattagggagaagagttcatgagctgtaagtttagataaaatatagtcggcactcagtacatttggttgcgtagttgagtttggcataaaaaaaaattttttttttttgagataaagtttcactcttgcttcccaggctggagtgcagtggtgtgatctcagctcactgcaacctctgcctcccaggtttaaatgattctcctgcctcagactcccgagtagctgggattacaggtgcccaccaccacacctagttaattttttgtatttttagtaaaaacggggtttcaccatgttggccaggctggtctcgaactccttgacctcaggtgatccacctgcctcggcctcccaaagtactgagattataggcgtgagccaccactcccagcctggcataaaaatttatgtctggcatacagtcactgttcagtaaatgttgagttgaattgaatttatttatttattttgagatggggtcttgctctgttgcccaggctggagtgcagtgatgcaatcatggctcactgcagcctcgacctcccaggctcaagtgatcctcccacctcagcctcccaagtagctgggactataggcacgcaccaccatgacaggctagtttttgtatttttgtggagatggcatctcactatgttgcccaggctgatctcctgggctcaagcgatcctcctgcctcagcctcccaaagtgctgggattacaggcatgagccaccacctgtaaggagcttatagcctattatgagggaaagatgtacaaactggaaatttacaaacagggatggttagtattgagatagtgggatctcaggaggctgtgggagctggaggcagcccctacccaggttctgataggatataacacataagctgtgttcagttcaatttgatttcattctgttcctttacattcagttgactagagttggaatcagtctcactctcatttgaattgagttgaggtgaactggattaaattaggctataaggagcttatagcctattatgagggaaagatgtacaaactggaaatttacaaacagggatggttagtattgagatagtgggatctcaggaggctgtgggagctggaggcagcccctacccaggttctgataggatataacacataagctgtgttcagttcaatttgatttcattctgttcctttacattcagttgactagagttggaatcagtctcactctcatttgaattgagttgaggtgaactggattaaattagattgtttagttcaaccataatcagtttagttatatttatttctatttggttgacttaagttaaaattgttcttgacggccaggcacagtggctcacgcctgtaatcccagcactttgagaggccgaggtgggtggatcatgaggtcaggagatcgagaccatcctggctaacacagtgaaaccccgtctctactaaaaatacaaaaaaattagccgggcgtggtggcgggcacctgtagtcccagatacttgggaggctgaggcaggagaatggtgtgaacctgggaggcagagcttgcagtgagctgagatcgcatcactgcactccggcctgggtgacagagcgagactccgtctcaaaaaaaaaaaaaaaattgttcttgacaaattagtgagccaggctgggctcagtggctcacacctgtaatgtcagcactttaggaggctgaggcaggaggatcccttgaggccaggagctcgagaccagtttgggcaacatagcgagaccctatctttgcaaaatatacaaaaattagccaggtgtggtggcgcacctgtagtcctggctactttggaggctgaggtggaaggatcacttgagcccaggagttcgagtctatggtgagccatgattgcaccactgcgctccagcctgggtgacagaatgagactctgtctcttgaaagaaaaaaaaaaaaaatagagagaagaacaagttggttgattgcattgagttttattgagtacaaattcattcatttccatttagttcatttacattcaagtcaagttgttctcaactgggttaaattgtcttgagttaggatgaactgggttgttggattagatgcaattgatttcagttgaattgggttgaattgggctaatttcagctgattatattcatttccattgattaacaaaggtgggttgaattgccctgcgtatgttcagttgggtttagataggtagaatcggattgggttgacttgggtagagttgagttcgagtcgatttatttccattattttgatttgggttgatgttgatcttggacaagttgattaggttcaatccaacggaattcatttgagtttaattcaatccagttaggttgaattgcactccattcagtgtgattggcccaagttcagttccattgatttaactcagaggaagaacttacttccgttaagctaagtgggcttaatttgcattcatttcatttccatttggtttagtggagctgggtttgttcaactaaatttggtttcatttatttccattcacttttagagtggagtctaaaaatcttaagctttatcaagctgcattgacatgagctctgtttgggccagtttatgagttcaattcctctctatatagatttcttacctgaaccatacacatgatgatgggatgcattcctctaaaacctcttgttcttcagtaataaactgaccgaggcaccaacctctggaccccctataaccgaggtctccactgtccatttcaggccaagaagggggaagttctcccagaggaacaggggcccagcatcaagaacctcgacttctggtccaagctgattaccctcatagtgtccatcattgaggaagacaagaattcctacactccctgcctcaaccagtgagttgtgtatctgtgtagttaattatctgggtgtcatcgtgtgtatgtatacaactttgtgtcttgtgtgtcaagatgattttgtctaattacacaaatgtgcacctgggggtaattgtgggtctgattatacctgtgcaaaattcaaatatgggtacaatttgcaaacataaacataaaattgtgtgtatgttcacaatcactggtataatcagacatgtattttgtatgtattcatttgcatgcataactgtgtgtacatatgtgtataattttttatgtgtatgtaggttaattaataatagtgttgctcaagtattactatgtgctaccctaatacatatcatacatttaaattgcattatatcttatatgtatggatgtaagtaattttgtatattgttgtggtcagtgggttttgggtttaatttgtatatgtgtaaaatcattgaatgcaattcaacgtacaatattgtacaatatacaatattgtgtattgtgtatgaatatgggcacagttttcatttgtggatttatatgtctgtagaattttcatctgtgttagctgaatgagctactgtgttctatttctctgccagatgagtgctagagaggactctagctttggggcgttaacatatcctggttagggaattaagatcagggatttggatggattgaagggtgggtggatgaatggatggattaataaacagatggggtcaggcacagtggctcacgcctgtaatcccagcactttgggaggccaaggtgggtggatcatttgaggtcaggagtttgagaccagcctggccaacatggtgaaaccctatctttactaaaaatacaaaaattggccacgcgcggtggctcacccctgtaatcccagcactttgggaggccgaggcaggcggatcatgaggtcagatggagaccatcctggctaacatggtgaaaccttgtctctactaaaaatacaaaaattagccgggagtggtggtgggcgcctgtagtcccagctactcgggaggctgaggcaggagaatggcgtgaacccaggaggtggagcttgcagtgagccgagattggccactgcactccagcctgggcaacagagtgagactccgtctcaaaaaaaacagaaaaaacaaaacaaaaattaaccgggcgtggtggtacacgtctgtaatcccagctagtgggagactgaggcaggaaaattgcttgaacccaggaggcagaggttgcagtgagtcgagattgcgccactgcactccagcctggggtgacagagcgagactccgtctctaaataaataagtaaataacagatggacagatagattggtagaaggatgactgtgtggataaaggaacggatgggccaggtgtggcagctcacgcttgtaatcccagcactttgggaggccgaggcgggaggatcacctgaggtcaggagtttgagaccagcctggccaacatggcaaaaccccatctctactaaaaatataaaaaattagccgggcatagtggctcgcgcctgtaatcccagctacttgggaggctgaggcacgagaatcatttgagcccgggtggtggaggttgcagtgagctgaaatcgcaccactgcactccagcctgggtgacagagtgagactccgtctctaaataaaaaaaaaggaatggatggatgggtggacagatggatgggtaaatggatgggtggatagctgcataagtgagtaaataaatggatagagggatggatggataggtggatggatgaatagatagatgggtcatttaatgggtgggtggatagttgtatgagtgggcgaatgaatggatggaaggacagataaataggtggatgatggaaggatggacggatggatggatgaacagatagacgggtaggtggctggatggacagttggatgaatgggtggagggatagatggatgaacaggtagatgagtggttgagtggatggatgaatagttggatgaatgagcaaatgaatggatggagggatggatggatagatgaatagatagatggataggtgaacggatgggtggatagttgcataattaggtgaatgaatggaaagagggatggatggatggatgggtggatagatgaatagatagatgggtaggtggatggatggatagttggatgaatgggtagagggatagatggatgaacagatagatgggtggttgagtggatggatgaatagttggatgaatgagtgaatgagtggatggagggatggatggataaatagatagatggataggtgaatgaatgggtggatagttgcataagtaggtgaatgaatggatagagggatggatggatgggtggatggatgaatagatagatgggtaggtggagggatagatagttgcatgagtgggtgaatgaattgatgaagggatggttggatgggtggataatgggtggatggatgaatagatagatgggtacatggatgggtggacagttgcatgagtgggtgaatgaatggatggaaggatggatgaatggatggaggaatagataaatgagttgttgaatgtatgggtggatagttgcatgagtggatgaatgaatgaatggagggacaaatggatgagtgggtggaggctcaaagggaaacaaagccttagtgcagagtcagtcagcagtggctatgagggaacctgaacccaaatccctctaccctaccctacactcctctgtggggcagggaaggacaaagcagcccatcccatctgccttctgcctgccaaagttttatacttccatgactttgccaggtttccccaggagctgaatgtgggtaaaatcagcgctgaagtgatgtggaatctgtttgcccaagacatgaagtacgccatggagggtgagtttcccaccccagtgttccccagcctggccacaccaggtggctgtacctctgtgtcccctccaaggctgtgagcaccctataataccctcatgcctgagttggaaatcagtgatggccagagtaggcagcctctctgccgagacagagggtggcagacccttccagctgtgccatcctctttgtccctggccagagcacgacaagcatcgtctatgcaagagtgccgactacatgaacctccacttcaaggtgaaatggctctacaatgagtatgtgacggaacttcccgccttcaaggaccgcgtgcctgagtaccctgcgtaagtcccctgccccaggcccaacccaagccagaactgtagcactgacagctagaaggaaagccagagaggaagactgacctacttagggttacctgggcatcaggggcatgggtggggcttgaatccagtgttctccactctcagccattgagcagttgactcctacagcccagaaaattcagtcaacagcaacaggaaccatgtgactttctcattttatccttttgacaattctatgagttaaggtgggatcattttaattcctattttgcggaggagaaattggagagttaaagatgccaaatggccaggcatggtggcacatgcctgtagccccggcaatctgggagactgaggcaggacttgcttgagcccaggagttcgagaccagcctggaaaagatagtcagatcccatctctaaaattaatgaatgggtcaggcacagtggcttgtgcctgtaatcccaggactttgggaggccgaagtgggcagatcacctgaggtcaggagttcgagaccagcatagccaatagggcgaaaccccagctctacaaaaatacaaaaattggccgggcgcagtggctcatgcctgtaatctcagcactttgggaggccaaggtgggtggatcaccagaggtcaggagtttgagaccggcctggccaacatggtgagaccctgtctctactaaaaatacaaaaattagccaggtgtggtggcagatgcctgtaatcccagctacttgggaggctgaggcatgagaattgcttgaacccaggaggcggaggttgcagtgagccgagattgcaccactggattccagctggggcaacaaagtgagactccgtctcaaaaacaaagaaaaaaattatccgggtgtgatggcacacacctgtagtcccagctactcgggaggccgaagcaggaggatcgcttgaactcaggaggcagaggttgcagtaagcggaaatcgtgtcactgcactccagcctgggcgacagagtgagactccatttcaaaaaataaataaatataaaaataaaatgaatgagtgaataaatgccacattccttgcccacggtgactcccagtacatgacagacccgagatttgaactcgggacatctgattcctgggctgtgtgttggaggatagtacggtgccgccggttcctgagaattgtggtgtcgttctctgctcccagcagcacaggaggatggctagtcttctgttgtacaagaaacgaaagctctggctggatgcggtggctcattcctgtaatcccaacattttgggaggccaaggtgggaggatcacttgaggccaggagttcaaggcctgcctgggcaacacagggaaatcctgtctctacaaagaagacaaaaaaaaaaattagccgggcatggtggcatgtgcctgtagtgccagctattcgggaagctgaggtgggaggattgcttgaacccagcaggtcaaggctgcagtgagctgtgatcgcaccactgcactttagcctgggcaacagaacaagacccaggaaggaaggaaggaaggaagaaggaaggaaggaaggaagggagggagggagggaaggaaggaaaggagggagggagggagggagggagaaaacaagagctcagagaataaacccacttgccatgggcagtttatctgaagaaggaaggaaggaaggaaggaaggaaggagggagggagggagggaaggatggacggaggactcagagaggactcagagaataaacccacttgccatgggcagcttatctgaactaggattcccacaggcctgggccctgttgtaaaaggggccactcaatctatttcatgagcaaagtaactgaggttcagtgcaggaggcgagcagaggagcagccactcaaggctgcagggagagctccaggcagagcaggagcagccatagctccactcgttagtgggtcagaaccaggcggcgcagaggttgaggaggacaagaggtggcagagccccaccccggctcttcattccagatggtttgaacccttcgtcatccagtggctggatgagaatgaggaggtgtcccgggatttcctgcacggtgccctggagcgagacaagaaggatggggtaagaaggcccacccacaaccaagttcctaatcttcccactggggtcaatttaggtggaaatttgcccttgagatgaggccagtctctggtaggtgtccgccatctttgtcttgctcatggtttttaaaatcttttgttcattcatcaactacaaaatgtgagttccatcaagccggacacctccaggcaccccatggcttctgcgtccacctctagagttgactctaagtctacctcgccctctgtccatctcaccccggggaaattggtctctggctctccccgtggagagtgggagagttgggctctagggattctctggtgctgatctcctccttcctcttggcctccttccccctgcagttccagcagacctcagagcatgccctattctcctgctccgtggtggatgttttctcccaactcaaccagagctttgaaatcatcaagaaactcgagtgtcccgaccctcagatcgtggggcactacatgaggcgctttgccaaggtgtgggagagtgggtagggttggggctaggggacagggtcagggttagggaattgaagtctgagttggagatgggctaaggttgaggttgaggttggagttggtgatgtcattgaggttgggattgaagttggagtttgggttgaggttggagttgaagtttgggttgaggttgaggttgagatgaggttggagttgaggtttgaatggaggttggagttggggttagggtttaaattgagattggagttgagattggggttggagttgaggtgtagggtaaggttggagttgaggtgtgggttgagatcagggttggaattgaggtcattagtgcgagttggcaatgctgtttggttggagtttgaggctgtgctgggtcttgatttggaattgagattggagtgagtgctgagattgatgcacaatttgagtgagtcttggggtgagtttgagggttgggatcattggggttgaggtttgattcattggcattaggctgtgttgaccttgggaattacaagtaggaattggcttttgatttgagatcaggtcatcttgtggttgaggctgggttaatatgatgcttggttgctctgaaaaagagctgggtctacataaaatgatcatcttcatcgagcggtatctcctctaggtcttgatggtcatcttgagtggagaatttgcctagcccaaaggcccatcttaggtcctaatagggtttgggttcatcactgatctcactagtggccttgatgggacgactggcgcctgccttggtgccagcctctcctctcacccacactctgtgtcttccccagaccatcagtaatgtgctcctccagtatgcagacatcatctccaaggactttgcctcctactgctccaaggagaaggagaaagtggtgacctgagggggcccctgacctatctccctcctcagccccattcagcatccccaggactcccttctccccagggccctgacacctggggctatgtatgtccacctgtgtgtgatctgactggattgtctgtgtgtttagaggtgcttgtttgaatgtgtgtgatcagagtgtatctagttgtgtgtgtctgagtgtatgggaagtaagtgcatgtgtctgactgtgtattgtctgtgttgactggtatactctgcgtgtgtgtgtgtgtgtgagtctgtgcacatctgagtgtgcgtgtctgatcatgttttgtctggctttgttaacagggtatggcatgaatgtgtgtgtgagtccaactgtgcacgtctgagtgtgcgtgtgtgattgtgtttcgtctggttgtgttaactgggtataacatgagtgtgtgtatctgtgtgattgtgtttcgtctggctgtgttaactgggtataacatgaatgtgtgtatctgtgtgtgtctgtacatatctgagtgtgggtatctagccatgtttcatctgtgttaaccgggtatagtgtgagtgtatgtatgtgtgagtatgtgtgttcaactatacaacacatctaggtgtgtgtctcatattttatctagctgtgttaactggttatagtgtgagtgtgtgtatgcttgtgtgtgtgtccagctgtgcatgtctgggtgtgtgtgaccgtgttttgtctggctgagttaactgcatatagtgtgagtgtgtgtgtgtgtttgtgcacgtctgggtgggtgtgtctcaccaggtttggtatggcggtgttgaatgtgcgtggtgtgagtgtgtgtctatggctgtgtctggctgtgcatgtctgagtgtgtgtatctgtggggggggtctcgtgtgtatatctcggggtgagggcacacgccttcagtgtgtcactgtctggctgttgcaaatgaagccttcagggtgtgagctgcaaagctttgggctcttgcaggaaactctgctactcccagagttcccagcccctctcatggaccccccaccaggcctggggtcgtgggattacttgagtcctccttgcccccaccagtccccagagatgcccgccccaacaacacgcccaggaacctgagcacaaaagctcccttccatttgcaggtgaccccatgggaccccagcttcctccagacccagaccctgaaaccatcccaaggtccctggcggtgggtaggggaggctctggggagaggtcctgaggcttgactcccctctctgttcccacccacagccctgcattctcatgaataacactcaacagctacgagttcagctggagaagatgttcgaagccatgggaggaaaggaggtgaggcagggccgaggcagggctggagaagggatggggcacccccaccactgtgtccctgcaggcccctgagctcatatgccaccaggcctggggatgaattccagctcaaccatggccttaaacccaggcttgcagcataacggtctgtgcctcagtttcccctgcagccccttgggatccctttccctgggtcagatgagaaaatgcccagtacatgtggatacttactgaacctggcctggtgtcccttctccctccttagctggatgctgaagccagtgacatcctgaaggagcttcaggtgaaactcaataacgtcttggatgagctcagccgggtgtttgctaccaggtgggagcatctccagggctgggggcagtggggagggaacagccttcagccctctaagcctggggcatctggaggctcagcagatgtagaggtgaagattgcaggctcagagttagacaaggctgagttcaaatcttggctttactgcttactagctgtgtggccttgggtgagtcacttaacttctctgagccttatttttttttttattgctctgtaaatttggggtgatgacaataatagtacctgggtaggattttgagaatgaaatgaacaatgcgggcaaatgtcttatccctttgcctggttctgcagttactattttccactctttttttttttttgagacgaagtctcgttctgtcaccaggctggagtgcagtggcgcgatctcggctcactgcaccctccacttcctgggttcaagtagctgggactacaggtgcatgccaccacacccagctaatttttgtatttttagtagagacagggtttcaccatgttggccagggtggtctcgatctcttgacctcgtgatccgcccgccttggcctcccaaagtgcttggattaaaggcgtgagccaccgcatctggcctgttctccattcttgctcggttatgtgctgtgatgcaaatgatatgacctcaaggaccacagatcagcatctgtgtcctcatccagcccttcctgtggtctgagcccttcccttctgtgtccccagcttccagccgcacattgaagagtgtgtcaaacagatgggtgacatccttagccaggttaagggcacaggcaatgtgccagccagtgcctgcagcagcgtggcccaggacgcggacaatgtgttgcagcccatcatggacctgctggacagcaagtgagctgggccaggtggactgggctggccggggaggggctggggcagggccatagagacccagggactggctggagagggtgtgcagggaatgtttgctgggtggtcactggatagttggatggctgagtagatggtggcatgaaggggatggaaagttgggtgggtaaataattggaaggattggtgagtaaatatttgtgtggatgaatgaatggtggatgaataggtgacatgagggatgggtaggtgggtggacgcatggattaaaagataaatgtttagaaggatagagggaggatagatgttttaaaaagatggatgggtggatagttgggtaaatatttggatggattaataaatgttttgatgcatagatggattgatgcttagatggatggatgtctggcaggctggcttgatggatggttggatggacgatggatggatggatggatggatggatggatggatggatggataaatggttggatatgtggataaatatttggatggatgaataaatgttttgatgcataaatggatggacagatgggtggatggatgaataaatggatgggtgggtagatggatggatagataggtggataaatatttggatgaatgaataaatgttttgatgtatagatggattgatgcatagatgtctgtctggctggctggatggatggatggatgaatggatggatggataggtcaatagatatttagatcattgaataaatgttttaatgcataaatggatggacagatgggtggatggatgaatggatggttggatgggtgggtgggtagatggatggatgctggatagatataggtggataaatatttggatggataaataaatgttttgatgcataaatggttggacagatgggggggtgggtggatgagtgggtcgatggatggatggattaataggtgagaaatatttggatggatgaataaatgttttgatgcatagatggatggacagatggatgaatggacaggtggatggatagataaatggataagtggatggatgggtagaaggatgcatggaaggatggatggatgaacagatggatggatggatggatagaaaaagaaatagagaattaaggaccactgggggagggatggattggtgggtgactggatcagttggtggatggatcttggtggactgcctgtctccttcaacccctatccatccaaccacaatctctttgctgttttccctttcaagtctgccctcctctgaccattcccctcctgttcctcttgggcatggccttctccctcatagtccctgatctccatccttcctgtttcggttcatcccccacactgttctttcaaacatgaaagtctggctgtgtctccctcttgaacactccatggctccccactacccccatcctgataaaacccaagccttcctcccagacattggggccccttcccatctggtccctgctgactagtccaaccaccactcactcttctcttcatgcatcagatatcatagccccatcaaaccacccaggggtccctgtacaggctgtgggccctctttcctatctgtggaatgccttgcccacctgttaagggaaggtgatctgtgggtgggggcgagctgggccctctctcagacctgcccctcgtccccagcctgaccctctttgccaaaatctgtgagaagactgtgctgaagcgagtgctgaaggagctgtggaagctggttatgaacaccatggagaaaaccatcgtcctgccgcccctcactgaccagacggtgagacctgcagggggcccgaggggacatttaggccacctccctggcgagagcccagaaaacttggtgcctagaggctggggggtaagaacaaaggcatccggtctcagagaggtcatccaggctcaagggccattcaagggtcatggaagccaccagaggtcagtggggggccattcagaggtcagagagttcacacaggggttaaagatcatcgaagagttaaagaggtcattcagagtccattgtattttctctggggtcaaagacatcaggtagagtcaagagaccactaaagtcatagaggtcacatgtaggtcaaaatagctttcaaaggtcagaggtcatctagaaaacaggtcaattttgggatcaaggttatccttgagccacggaaggcatagacattggccaggcaccgtggctcacgcctgcaatcccagcactttgggaggctcgaggcgggcagattgcttgaggtcaggagttcgagaccagcctgggcaacatggtgaaatctcgtctctactaaaaatacaaaaattagctgggtgtgatcctgtgatcctggcttcttgggaaactgaggcacgaaaactgtttgaacctaagaggtagaggctgcagtgagctgagatggcgccactgcacactccagcctgggcaacagaacgagaccctttctcaaaaaaacaaaaaaaaaaaagagaaggcatagacatcaatgggtttttctaagggctttggggctcattcaaaagccaaagaggggagacaccatggacttcagacatcagagatcaatagaccaaggggctcctggaagtcagccagaggtcatggagggcatccaaggtcagagaggtcaagcagagatcattggccatccagagaccaggaccgatagaccagaggccataatctctagggaccaaggggacccaaacatctgtagagggatgtagctgggcgtgggtccctccgggaggcatcaagaacctgaggacagcaatggcatgtgggctgtgggcacattagggagtggggaggtctttcccaggtggaccacggaggcttggggagggtgagcgggggccatagatgggcgacgatggtcggggaggctcggacttcccatccttccacctcttctgggctgggctggggcaggggtcttgcccctccgtgctgccatctctgccccctccctggccttcccgggagactcatggtaccttattcccccctcccccaccgccccgcccccatcacggatgacagatgatcgtaagtagagtcccgtctgtccgtctgtctgtccgtcctctctgtgtctggactcggcccaccgcgtcgctgcacccctctccccacccacccagcgccccctcctcacccaccatcctccctcccgcctagatgtgcctgtgtctgtctggggcggggcatggggagggggcggggctgggagggagggagaccacgcccaccctccctgtcccactgaagtccagaggagccccactacccactcccaaaccccaagcccagctcctccttcgcagcggacctgccgtctaagggtagaatccaaccttgctcccgtgcccagatgtcaccagacgccctgaagctagaaagaggatcaaccccaggattcctctgacctcctctttgggctctgaactgtcagcagcttcccagaatgcaggtcctcaccatgagggtctctacttctgattcctctgaaattccatgctccagaccattccttggtaccccaccaccctgtctacccaactcccgccatccacaccgacacacaccaatcccagacatacacccaggtgcccaagtgggagacttttgtcatggctttgatgtcaggtagacttggggtcaaatcctagcccagccactgtctctctctgtgaccttgggaagtcacttgacctcccagcctctgtttccttgtctggaaaatggggctcattgcatcacctccctgagggtagttctaagaaccaaatggcatgatgcattaaaatactcatctcagtattgcctgaacccaggaagcagaggttgcagtgagccgagatcgcgccactgcactctcagtgaccggcccagaggcagagctaatggtggtggtggtggtggtggtggtggtggtgatatgcagacatgtgtaagagcagagacaaaacaacttcagattttccctttgtgggcaagacaaggctgttacaagatttcactttgttaaaagtttacaagggctcttacttcctggtagctgcagcctgggtctttggctataaccacttggtggcagcatacccaaccacaggcaatgcatttgtaggcagggggaataaaggaattcgcatttaatgactccccatatgccaggccttctcatagctcctgtttcagctcaccttcatgataaccctttgagtgaggtggatattattatccccattttgcagatgaggaaagctgaggctcagagaggagtcacttgttcaaggtcatatagtaaatatatgccagaatcaggattcaaattcagatctgtctgaatatgcagcccatttgctttcctgttaacccaagaaccaagagtctcatgtgtacagtaaatgtaaatggattatattttggcagtggcttttttcccttctttctgactttctttagctctgtatctgtatctttctctctctttcctgtctctgcctctcacaccatttaagagtgagtcactaaagcgcatgcacgtgtgtgcacacacacagacacacacacaaacacactatccccatgcaacccagtcaccagtaccctgtgcagtgtggattcccccatccccatgaatattcctgtgagcatcccagcttcccaccctgcacgacttgccaggttgatcaccctgtgacattatctctgcaggggaacctcttgagaaaacatggcaagggattagaaaaggtaatgagggcctcgtctcactgaggccctgagctccagtgtctgggcatgtgtgtgcatgtgtgtgcgtctgtgcacctgggcaggaagggcagctgggggtcgtgggtaggagatagctctcctaaccaagtcctgcaaaaccagaagtcagagatgaagtgattctccagggacagaacgtgcagtgtgtgtgtccacatgtgaactaatgtgtgtgcacaaacagctcccatcctcccacagcaatacccacagggctcatctgagccctgggctcagctgaaattctgtgccttcttgaggagcctacacgctagatcaccaccctggactgagacgtgttgttttttacgtagatttctttttgttgctgtgctaatgggtgtttgttgttaaggagatgggagctacagcaacacgcaagcagctgtcttgctgtacacttgtgcagaagatatcgttgtacatttgtgtctacaaatggagtcttagctttgtgaatgtgggtcacaggggttccgtactctctctgccagccctttgcggggcacattcagtgtcccaggcaccctatatgtggatgtgaatccgtgtggggtcaagaagggtgtgtgggtttttgtgtgtgatataattgtgtacgtgcctgcaagtgtttaggagtgcacgtatgtagtttagtgtatgggttcttcagcactgagtgaacttacagtatatgttaatatgttttttgtagacttgggtgctgggatataggggcagccccttagggaatggaggaggagacagattcctagccctgtctcatctccccacctccccaggtgccccatcctggggcaggctgactgtgggatgaggctgcttccagcctcctgggtaccccgagagtgtgctggcagtcaaggagtctgacctctaaacccctgctaggggaccagaggaggtgctagctggggagggggcccaccattagacccaggacctggggaggagctcctgggctgggaagtgagactgtgctttgagggggcagggaagtcccactgagtatcctcccaacagtcaccagaactctgacctccacctgattctctccacagggcagggtgaaattgccaagccactcagacgtaagaccggcctctcactgtctgtctggctcccatcccactcccccccaccccacccctgtgcccccttccacctctgtggttcgtgccgtccgagggcgtactgtgaacctgtctgttggtctcttcttcctttgtctgtctggcttgctcgtggggggtggtgggtggggggttggtgggtagggcagagggtacccaggaaggcccctgagccctgccagactacaggtgggtgcagcaggggaaactgaggcccagtccgctggacccctgagtaactgtgccttgtaagaacctcaggatcacagacaagaaataagaatagtaacagccaagatgataatagtaatacctctaggaatgaccagatggccagctggtagactatgaacataattccaaatatttccacagtaaatgggccccctccccacctagcctcacccaggacccccagaccaccccaggacccagtaagacaagacttgttacctcaatactcagccccaccctgtcccccaagttaccatgggtatccctgttagccacagtgggaattgaggctcagagcattcagggacaagcctggggccatccagcagtgacagaggaggctagacacatgccttccaggctgggaccccgtctcccttagcattgcccgtgactttgagaccaagtgataattagcgaacactgagcaaagacttgttcagtccagactaagccacaaagtggatataattttcctcatctagatgggaaaactgaggctcagagaggttaagtgccttgcccagggccacacagcaggactgcaatttgaacccaggcccatctggcttgtcagagccatcaccacatcaccatcttgctagtgtctgtttgggggtggggtggaggacggatatggaggtgtttgtatagagggaggatttttcctttaaaaggtcagactggctggcaatgtggctcacgcctgtaatcccagcactttgggaggctgaggtaggaggatcgattgagcccaggaattcaagaccagcctggacaacacaacaagaccccatctctatgaaaaaaatttcaaaaactagctggatatggtggctcacacgtgtagtcccagctactggggaggcgggaggattgcttgagcccaggagtttgaggctgcaatgagctatgatcatgccactgcactccagcctgggcaacagagggagactctgtctcaaaaaaaaaaaaaaagaaaagaaaaatcagactgaaattcctcccagattacaggggaaatttccctgcagaggggatgctccttgaggaggaggaagtgtttgaccagagccattttctgcaacctcagcctagcctattgtgttcccaaagctccctttccctggaaaccctgcccacacccacaaacaccaccgtctgaggctcccccaacccactgcccctcacagtctggtccctgtctgtttctcctgttacacaagcctgggtggggctgagagggtgggggactatcctcacaggaccgggattttcagggacaaggtcaccacatcctcctcagttcttcctggggaattccagacctctgtccctgggacacaattattgccctgcccagagcctttgggcaagaaccaagatggtctctggggctggagtgtcagctgtagcacccctgccctcacccagctgtatgttgttacctgcagggaacccagatgatcttcaatgcagccaaggagctgggtcagctgtccaaactcaaggtgaggagtggggatagattatggcgtgagggactgccccccaaactcatgtggcttccaggttgccaccatcctgggggaggggtcccaggatgatgacatgcattacaggctcaaaggacagaaactctactcactgaagcacaagatatttgctccccagtgtgtcagaaagatgtgaatggttcagcttcaggtacagcttgatcaagagggcaagtcatttattcaaccaatattttttgagcacctgctgtgtaccagtcacatattgggtgctgggatgatgaaaacatagaccctcagcccagctcctccaagttgaacagggatgagacccatagacacagattccaaaggtggagcccactccacccacatggcccctctccccatccatccaggatcacatggtacgagaagaagccaagagcttgaccccaaagcagtgcgcggttgttgagttggccctggacaccatcaaggtgagaggccctacccctccccatcccaggtgggggatgtagagggtgtggctgctgtggtcatggacttaggctcgcctcggacccacattaatttgttcaatttatggacactccctcgttcccctgtctgtgccagagctgggctggctaatgctggagccttaaggagccccaggtcggggaagactgctggatacagacattctcagccttgtaggattaggcttggggtagagggaaggatgtgggctgggggagcccaaagagtgagaaggtgtagagggaaggatgtaaaggacctcgtgccaggcatttcttgaggtgtagaagggtagagggaaggatgggggctgggcgagcccaaggtgggagaagccaggccttcctggaagacggcatgtgagaggtgggttctgaggaatgcataggagttcactaggcaaagcaaaggaagaggtgagaaagtagtagcgggaagaattgggagtggggacagcagggctacccagccctatgagtgggtagaggttcccaccatcctcacctctccccaccctgtcctccccagcaatatttccacgcgggtggcgtgggcctcaagaagaccttcctggagaagagcccggacctgcaatccttgcgctatgccctgtcgctctacacgcaggccaccgacctgctaatcaagacctttgtacagacgcaatcggcccagggtaagggtacccagaccgcatcaccctttcccgctccgcggagacagccccgccccaagcctgaacggctgccattggctcactcccctgtgacgtcatggatggggcggaattgacatctctgcaccaagtccagggaccaatgagcgatcaaaggttcatgggggcggggcctctgacgtcacaggcctggaggtgatcgtcaacccctgggaaaatctgtgatgtcctggggccagacaatgattaacagctctgggatggaatgtctttgacatcaaggaccagggtaggattgatgactcttaggtgtggaacctgtgacgtcgtagggccacaaggtgattgtcatctcctgggaaaatcaatggtgtcccagggccaatgaatgattgtcagctcctgagggacctggacagtcatgggtccagggaatcgttgacagctctgggagacttcaggagtgattgacagctcaggatgggcaggacctctgatgtcaaggggtcaaggaatgattggcaggtccatgggtgggaactttgacgtcatggtcccagagagtggttgaactcccctgaggagggacctctgtgatgtcatggggctaaggaatgattgacagctccgagggcgggagggacctcgacttggtgggggagggtggacggagagggagttcccatttctcaaactctctctttctctctgtattttgcttcggtctcctgtccctctctgagcgcacacacacggcagctggggctggatggagggcaggattccctggggacccccccttttcaaaattggtcccgacaaggggaagaagtttggcggcttgaactcaggagggggagggagaatcgccatcgacacccagcctggggcgaggggagcaaagccctgtggcctccaacggcccccttctcccccgatccagccagcccaagggccctccgtgggcgctgggtctctgagctcagcttcccttccagtcctggctgcttttagtgtccctggtgtccctggagcaggaggctgtctgtctctccgtctgtctgcagctgacgtttctctctctcttgtttttcctccctctcactcgccgtcccctctccgcgcctcccctgccccctccacacacactcctccatcctcacctctcactggctcgtctctccccgctccccatgcttcccacctctctcccccttccacccccacccccaattagtccatggtggaaaaggtactaggtttacccttagtgaagacatttatcctgagaagggtacgtgcctccgcctcccgcccgccgcccgcgcctggtgcccggtgcctgcgcccctcctggccccggctagtaaaaggatcgtccttctgcctcaagggcctgcagggccgccgcccaggcctgagcggcttggagggcccaggcgggtgagggggaaggacgatttgctggttctacatggggaagaagcgattccagtgggaatcccagaatccatttctcctgatatgacatattttttaagccagatcctgggaccccgggacaggcacctgtcaggttaagagctgggctctgattccagcccagccactccacgggtaggcaccttgggcaagaatctgtctccctgagcctcagttttctcatctgtaaaatggggacagtcaaggaccctacttcataggattattgttattgatattattatttagagacaaggtctccttctgtcacctaggctggaatgcagtggtgcaatcactgctcactgcagcctccctcctgtgctcaagtgatcctcctgccccagcctcccgaggagctgggactacaggcacacgtcacatggctagctaatttttaattttttttgtagagaggtggcctcactttgttgcccaggctgatctcagtgaatacttatcaggctctaaggctgagcaggaccttgtgccaggcatttcttgaagaatctgtgtttggttttgttgttgttgtttgagacagagtctcactctgtcacccaggctggaatgcagtggggaaatctcagctcactgcaacctccgtccgcctcctggtttcaagcgattcttgtgcctcagcctcctgagtagctgggattacaggcccgcaccaccacgcctggctaatttttgtatttttagtagagacggggttttgccatgttggccaggctggtcttgaactcttggcctcaagtggtccacccatcttggcctcggccttccaaagtgctaggatgacaggtgtaatcccagcgcctggccattttgtttttaatttgaggcagacgctgtcacccaggctggagtgcaatggcgaaatctcaggtcactgcagccgccacctcctgggctatgttgcccaggctggtctcgaactcctggcctcaaatgatcctcccaccttggcctcccaaagtgctgggattacaggcatgagcctccatgcctagccttcttggggaaactgaaacagatttctttaactgccccacgaggggtacatcattccacagatgagatactgagagccaaaggagattcaaccacttgtccgaggaaccttcccctgcaggggtcccgggacttggggagtggggagtggggttggaagaagcactgagaggcccaggaaagacccttccccgcgaagccagcaaagcacgggatgggggcacccagcactgcctccctccccccttcttactagctttggccttggtctgacccctcacctgacccaccactgtccaattccccctggcacccccggcaccccagtctggcccggattgagcttgctatgaagcatcggcctgggggagggcggggcggctgagcatgtgactggtggcggggggtgggggtggaatgggagcctgaggcaacaaggaagacggcctggggcgtgcagcaaagaccggggccacataccccagtactggggtgatgggggtctccaggcccctgtgctccccaaccctcagtcacttcctgtccagcgtgaggtaacccaatcagcgtttgactgacccaacctcccaccctcacctctgactttccccttcttctccccgtgcatggggtggtccaggagtggggagttgcctggtggggggtggggaggggttggtggaggtgctggcggtcacaggtccacaggccccacaggatgcccttttgagtcacagaaggcgggaccgggagagattcttttaagctgccccatcagtgatccccattggcaccatggaggacacggctggtggccccaaacagaagcccaccacaccatgattcaagcagacaagggaactttttgactccataatggaaaaattaggggttgaggcctggcgcggtggtgcatgcctgtaatcccagcactttgggaggccaagttgggcggatcacttgaagtcaggagtttgagaccagcctggccaacatggtgaaaccccatctctactaaaaataacaaaaattagccaggcgtgttggtggatgcctgtaatcccagctactcaggaggctgaggcaggagaatcgctttaacctgggaggcggaggttgcagtgagccgagatggtgccattacactccagcctggtgacacagcaagactctatctcaaaaacaaaaaagaaggccaggtgcggtggctcatgcctgtaatcccagcactttgggaggccgaggcgggcagatcaccaggtcaagagatcgagaccatcctggccaacatggtgaaaccccgtctcaaaactacaaaaattagctgggtgtggtggtgtgcacctgtagtcccagctactcaggaggctgaggcaggagaatcgcttgaacctgggaggccgaggttgcagtgagccgagatttcgccattgcactccagcctgggcgacaaagcaagactccatctcaaaaaaaagaaaaaaaaaaaaaaaaaaaaaagaaacttaagattctgcactagaagggaaataggggtaaggaaaataaaagatgccgctatgagctgtcacatcaggagggatgaggattctgggccaggtgatgcaatgaggtgacctgtggggctgggtcagagttaacaagggcaagaatggggtgagtggggagaggtaatagtaacctattggaaagggccactattggaaggggtgttgcaggtcacaaaaaccactttgattatctcagagtgaggtgggagccatggaagagtttggttttgctttgttttgttttttgagacagtcttgctctgtcacccagactgaagtgcagtggcgggatcatagctcactgcagcctcaaccagctgggattaagcgatcctcctgcctcagcctcctagatagctgggactacaggtgcatgcatcaccacacctggctaattttcttttctctctcttttttttttgagacagggtctctctctgtcacccaggctggagtgcagtggtgcaatctcagctcactgcaacctctgcctcccaggttcaagtgattctcctgtctcagcttcccaagtagctgggattacaggcgtgcactaccacacccggctaatttttgtatttttagtagagacagggtttcgccatgttggccaggatgggcttgaactcttgacctcaagttatctgcccacctcagcctcccaaaatgctaggattacaagcatgagcctcccaaagtactggggttacaggcatgagccaccactgcccgacccatggaagagttttgagcagaggagggacatgatctgacttttaaatgttatttatttattttttaattttctgagacagggtcttgctctgtcacccaggttgaagtgcagtggtgcgatctcgtctcactgcagcctggacccttctgagctcaagtgttcctcccacctcagccaccagagtagctggggctacaagcacgccccaccatggccactaatttttgtatttattgtagagatggggttttgccatgttgcccaggctagtctcaaactcctgagctcaagtgagtttgcctcaccctcccaaaatgctgggattacagctgtgagccaccgcgccctgccgacatgatctgacttaataggatttttctggctggtatactgagagtagatgaaagagtggaaggaagaccagcaaggaggctactgcaatagtccaggcatgggatgctgggggcaggtgtaggatacagggaaaagacatggctctgcttctggatgtattttgaaggtagagctggcaggattttccagtttatgggcacctagggaggaagtgaagacaaagaagagtgtcaaatattgaggtgcagggtcagtttgaggacccacggaggagaaaatgacaaaagggggttggagggagtgacaactatgtaggatgagaatcgagttggtgacattgttgcagccaagaggacaaacagccacaggaggccagggtgccagatgctgccaagatgtaatgtaacatgaagactgaattttgacaacatggcaatcaatggaataagaaaaaagaaaagaattgcattaggctcagagaggttaagcaatctacccaagatcacacagcaaggcagtaaaaggccagaacccaggattcttggcttcccaagagcacctcccatcctggccccccatcagtgcctccctatcagggctgatgacctggtgccgccagcacaggtgggtgggaggccgtcaaggtccctcctggtgggtgggaaagaacagagctcacgctgaccatcctcccctgccctgcccaggcttgggtgtagaagaccctgtgggtgaagtctctgtccatgttgagctgttcactcatccaggaactggggaacacaaggtcacagtgaaaggtgagtggtctgaggtgggagggacttgctaggttctaaaacagaaaccaaactcaaactggcttaagcaacaccctttttttgttgttgttgtttaatggtccatgaaacggaaacgtccaaaggtggccaccttcaggcatggctggatccagcagtgtaaacagttttgtaagactgaatcccactttctccatctctcagccctgctttccttttcttggcttttgttgactagaacacagattgggctgtttttgacatggcttaaataagacagaattaggccaggcacggtggctcatgcctgtaatcccagcactttgggaggccaaagctggcggatcaggccaggagttcagaccagcctggccaacatggtgaaaccctgtttctactaaaaattcaaaaagtagctgggtgtggtggcgtgtgcttgtaatcccagctactcaggaggctgaggtgggaggatcgcttgaacccgggaggtggagtttgcagtgagctgagattgcgccactgcactccagcctgggtgacagagtgagactccatgtaaacaaacaaacaaaaatgatagaattggtttttctctgggataacagtttaagtagcaaccagcctcgagctgatatggaggcttcgcagtgttggtgatctgtgctgctgccatcttttgttgcattttcatccctaatgtgttgttgttgttgtttgtttgttttagaggccagagtctcactctgttgcccaggttggagcgctgtggtgcaatcacagcacactgcagcctcaaactcctgggctctagcgatcctcctgcctcagcttccggagtcgctgggattgcaggcatgcagcatcacacctggctactttttaaattatatttttgtagagatggtgtctcactatggtatccaggctggtctcaaactcctggcctcaagcagtcctcccacctgggcctcccaaagtgctgggattccagacgtgagccaccatacctggacccaaatgtgtttttcctccactatagggtcacaaagagctcagcccccacaagcattgcagactgcggctagaggaaggagaaagtttggggttcactccctgccttttaagccacagtggagaagtagtacacgtgatttttttctctcacctcattggccaaaacatagtcatatgaccacacttagcttcatggcaatgctgggaaatgtagtctttatttcagacagggtttttggcggcgggcgaggggtggggtgtttttattttttattttttttagacaggatcttgctttgtcacacaggctggagtgcagtggcacaatctcagctcactgcaactgccgccacccaggctcaagcaatcctcctgctttagccccacaagtatgtgggactacaggcgcgtgccaccacgcccagctgatttttgtatttttcgtagagacagggtttcaccatgttgcccaggctggtctcaaactcctggactcaagtgatccacccgccttggcctcccgaagtggtgggattacaagcgtgagccactgcgcccagccttgggcaggttttgagagccacaaacatctactttttccaactgggagaacctgacctgtcttaccctctacttcctccaacagtggtggctgccaatgacctcaagtggcagacttctggcatcttccggccgttcatcgaggtcaacatcattgggccccagctcagcgacaagaaacgcaagtttgcgaccaaatccaagaacaatagctgggctcccaagtacaatgagagcttccagttgtgagtttgggatgctttgaagagcatgggggcaagggagggggcatccgccaggtgaaccccaaagccagctaggcaggaggtatctggaatctgggcccgggaatgggaggggccacattctaggtgggggtgctggaaagggagaccccttagtagtgcaggaatgacatcagggaggggacacgaggatggaggtcacctttgggggctggcagatccaaggggaggcaatgagtagagtggggtaacttcatgatgaagacttgcagaggggagggaaagtctgggcaggaagtgcatggtgggcagggccattgctggggctggagactctgtctggtgaaggaatgagggtggcagtgatgacgcttatgagcggggggctgagggacctggggacaggactgaggttgcatgggggtggggactactggagtctacattgagccttgtcaggaccgttggggcagcccatctggaggagtcggctctggggacaggagtggagtgtagatggatcccaggcttgggggtgggtgtcacgtggggtggaggtggggtcacattggatggaggttcttggagttgggagtgggcctaggctgcgggtatcaggatagggtctagaggatgctgatgatgtggacagcataaaaaagtctgctctggccgagtgtggtggctcacacctgtcatcccagcactctgggacgccgaggcagacagatcacctgaggtcaggagttcaagaccagcctggccaacatggtgaaaccccgtctctactaaaaatacaaaaaaaaaaaaaaaaattagccgggtgtggtggcgcacgcctgtgatcccagctactcgggatgctgaggcagaagaatcgcttgagtccgggaggcggagggtgcagtgagccaagatcgagccattgcactccagcctgggcaacaaaagcgaaactccacctcaaaaaaaaaaaaaaagtctgctctggccgagcatggtggctcacgcctgtaagctcaacactttgggaggccaaggtgggcagataacttgaggccaggagttcgagaccagcctggccaacatggtgaaactccatctctattaaaaatataaaatatggctgggtgtggtggctcacacctgtaatcccagcactttgggaggccaaggcgggcagatcacgaggtcaggagatcgagaccatcctggctaacacggtgaaaccccgtctctactaaaaatacaaaacctagccaggcgtggtggcgggcgcctgtagtcccagctactcgggaggctgaggcaggagaatggcgtgaacccgggaggcagagcttgcagtgagccgagatcgcaccactgcactccagcctgggcaacagagcaagactccgtctaaaagaaatatatattatataattatatataaaatatgtatttatattatataattatatataaaatatgtatttatattatattatataatatataataaatatatatcatataaatatatttttatatattatatataaatatatatatatttatatacatatataatatatatttatattatatataaaattatatatagtatatattatatagtataaataaaattatatatagtatatattatattatatataaaagtatatatattatatatttcatatattatatattgtatataatatagtatatacaaaaaatattagctgtgtgttgtggtgggcgcctgtaatcccagctacgcgggaggctgagacaggagaatcgcttgaacccaggaggtggaggctgcaatgacccaagatggcgctactgcagtccagcctgggctacagagtgagactcaaaaaaataaaaaatacagaaattagccgggtgtggtggcaggcgcttgtaatcccagctactccagagggtgaggggtacgagaatcgcttgaacccaggaggcacaggttgcagtgagccatgattgtaccacggcactccagcctgggcgacagagcaagactctgcctcaaaaaaaaaaaaaaaaaaaaagtctgttcacgggggtagatggatggtggtggttagcaagggtagagttggggtcatccggggtagatccttgggtcgagtgtcagcctcacagagcactggggcagggcaatgttgttggatgttcttaggagacagaagtgcagaagaaagtagtcccagctacctgggaggctggggcaggaagatcgcttgagctcaggagtttaaggctgcaatgagctatgattgtgccactgcactccagcctgggtgaccgacatagcgagaccctgtctttaaaaaaacaaaaaactatgggccaggcgcggtggctgacgcttgtaattccaacactttgggaggccaaggcagtggatcacctgaggttaggagttcgagaccagcgtggccaacatggtgagacccccccccccgccacccccatctgtactaaaaacacaaaaaatcagcccggtgtggtggtgcatgcctgtaatcccagctactagggaggctgaggcagaagaatcgtttgaacccgggagacagaggttgcagtgagccgcgattgtgccactgcactccagcctgggtgacagagggagatgcccactcaaaaaataaaaataaataaaaataaataaaaatgcacaggggtaagggtaaatcagagctggttagctggtattatctagggggcctgggagctcttatgggacaagattgaagccagttccatgtcgtgtcagcacagggctcttggtcagaaggttttggggcgattggcccctttaggggatactcaggaggagaagcatggtctagtgggaggtcagatgtcagccagggtgggccggagtacttcagggatagaggtacaacctgggaggtggacaagagggagaagctgggcgtgaccctgtaggaggggctcttatccagctggcgtcagggcaggtcactgggactgcttaggggcaggagcacaggtgaggggcatgcgggaggcggtgtcacccatggtgaggcaccagggcaggctcatgtctggagggcatcttggaagtaagaggggtgggctgggggtgtggggggctggggaaagggccacgtcgtgggtctagaggagagcctggttaaagagcttctgggtgggacattaggggcagggccgtgcgggcagcagttcagccggcagagggcacaggcgcgggtatctcagggtgggagcatggccttgcagggcggtgtctgaggggcgggagctggatgtaaaactatgcggtcatttttcggtgggtggaaaaatgggaagaacaggcgtgggcgtggcccagctcggggtcagctgctgtgggcgcggccaagcacagggttgtaggtcaagtaacggggcaggggcggggctagcgtagcgtgggaggtgacaggtgtgggcaccccggtgtgggcgtggcccgcaaatgcggtggggacggggccgtgggcggggcatcacagtaggtgtggcctgtctcacgttccgcccctccccacgcagcacgctgagcgccgacgcgggtcccgagtgctatgagctgcaggtgtgcgtcaaggactactgcttcgcgcgcgaggaccgcacggtggggctggccgtgctgcagctgcgtgagctggcccagcgcgggagcgccgcctgctggctgccgctcggccgccgcatccacatggacgacacgggcctcacggtgctgcgaatcctctcgcagcgcagcaacgacgaggtggccaaggagttcgtgaagctcaagtcggacacgcgctccgccgaggagggcggtgccgcgcctgcgccttagcgcgggcggtcggccgagcggcactgcgcctgcgcggagggcgctgggcggggagggacggggcttgcgccttggtgggacctccccaggggcggggctcggggggctccacgccaagggtgggctgcgcctacgcccttgactcagctttcccttttggggaattaggaatggaggatgccccgccctctcgggaggccacgcccaagggcgcgacgaaggaaggagccacatccccaacttgaggccacgcccccagcacctagggggcattttgagctgggatgggggaaacctcgtccctatggaggaggccacatcccggggctctggtaccgggaggcaccacctcatgtcccctggaaaagccataagatgggacccagacccctgggaccccagaccaattgccaagtatggaaatctcagctccctcgagggggggccctgggcaaggggtagggctctctggagcgcccctctaggtggcctggggactggagggaccaggatgctggttggagggccccggaataccggagtccctttagatatttgtgcaaaaaataaatggggggaggggggaggatgggatttcaaaagcacatgcgcccttgggcgcccaaaccctgggggccgaggggacggctctggttccccacgctgcccctacttccctttgggagtttgcctctccctctcccccaacaaacccagtcctcatatcatagagttcaacacacccatttgacagatggcaaaactgaggcttaaagagctgcttgagacttggccaaggttccaggtgccataccctctgtgcccctcccttaggcctgtgtgccccatggaagggtgggctgagatcgggatgacctgacacagctccctattgctgctaattccccctcggcctcctccaaggggtgggaattccaggccaagacccctacttcgcctttccttctccggctgccaagcaggacctttgccctcagccctttctcctgggatctccatgggggatgccatgagggcctcccaccacaaaagagaatttgggatcccctggtcccaggtttctccatcccttcttccttttccagaattttccaaataggaaagaacagaaggagaccagaaactctaggggggagaaagagaatgagagaaagagaatgagagagagagaaacacaaacacagtgacacagtgagagcttagtctccaagagcctattcattgattcaaacacccaagccacaggatacctcagatggccctcttgccagctggaagctctttctccaatgagcaaagttacagtgacctggctggagttacctggtgcacataggaccttaggggaaagttcagcgtggactacacttgctctgggatctgcttttccacatgtgtgtatggcacgcctttttctgctggattgggaaggacaagattttgctgtgctagggagaaatgaaaacggggtgagctgagtagctgggtttctggaggatagaacatcagatggggaggctttccgaggtgaagaatgagagggaaccacttactagagagaaaagagctccaggcctggggaacagcacgtgcgaaggccaggagagaagaactgttgaaacaacgagaagggtggcacggctggagctgagccagcaagggggatcgtgaggagccttggggttggggagatctgcagaagcatcagaccaggcagggcctcgtacgcagtcctgaggagttttacttttattctaagacagttggggagctccaggagctgttttaagttggggagagactggattccagcctgcaaaagctgttttgtgaagactaaaaccagtgaggagaggtggaggtgctttggggacactgaaatggattcttggaaagattctgaaggctgtgttgaaaagacacctatagctgtggggacatgactataatcccagcatttggggagaccgaggctggcagatcacttaaggtcaggagtttgagaccagcctggccaacatggcgaaaccccatctctgctaaaaatacaaaaattagctgggtgcagtggtgcatgcctgtagtcccagctactcaggagactgaggcgggagaattgtttgaaccctggaggcagaggttgtagtgagtcgtgatcacacaactgcactccagcctgggcaacagaacaatactccattccctcccctctaccccaccaaaaaaaaaaaaaaatcctgcccttagatgagctctagggctgctgagtacagttgtcccagttgcacagtgcccaagggtttggcattgctaagaaggccacgtgcaaatcctagatattgagtgttgtatgtttgtgacgttggtttcccgacatgtgaatggcccaagtgtctggaagaagtggcgccactttctaatttgcttggagatgttgcatgtcccttaaattcagacaggtgcaggtaactggaggttctgaaccaaaggttaaaatgcaaattctcatacagggttgggaagttgtagccagggataagcttatgtgactgttatatggactgaggagcagatgtgaatttcgaaccatgacatggctgagggtaggggtcgggtggatggatgattcagggttgtaacccatagagcccaaaggggaagtgatctgtgacctggggtgagggtgatctggaagatttttggatggctggaaagaaatggggaagtcgagctgcctgagagagccaagttatttcccaaaagattccttaggagtctttctgttcaagacctccgtgtgtgtgtgtgtgtgtttagggttccccagcaatggcccaggcatgtgaaggaaacaagcttcttcagggaatatttgttgaatgagttttcctgactcccaggctagaactgtttttgcaatttccaccctcttttctttcccccagagaactcctattcgtccttcaaaacccatcacggaaacccctcttggagaaaaccctccttccttcccctcaggactttcccagccaccgtctctcctccagtccagcctgatgccatgggactgggggtttctctgtccagctctgtttctcccagactggggtctgaggactctcaggacccccaactttacctagcacaggctgggcacaagtgggtgacagggagtctacgcctagtggaattatgtattggggcagggtcagtgtgagaatacacatccgcatgcatgtctgtccatgtctgtccgtaccaaccttccccttccacacggacctgggcacataggaggtgtctgagcctgacacatgggacagagagtggacatggctgagacacggacagagaaaagacaaggagtccagggggctgaaagccttttgaaatcaggaagttcctgtattggcagaacaaagcccagagaggagcagggctttcctcaacgccacccagcaagtggacacagagcccggccttggatgacacctccagggttctgaaccctggacctcgctttatgcaaggagctggccccacatttccatgaatcggggaaacagcacaagaaggttggcctgtggcagggcaagggttaaaggggtgacattgagggatgcctcagagtcaaagtcccctgaccaagaggaatagagtagaaaacacagagacagagggtgagatcacgccccgatgaggacggagagagacagagatggagagagacatagaggtggaaatatacagagaaagataaatgcagagaccaaggcagggagtgtcgggggaagtaaagagggtgtcctgaagaaagaaggatctgttcactcttaccagtctgtcctcgaatgatttgcataaaatgaggaggtgcctgtccacacccccaattcctctctcaggccccagagcctgagacctcaccatgcccccatcagagatgcaaaaaactaaacacccaactagaaatccttgggacctctctcggctgggatctcagagcctttctgtcccctacccctaccccatgtgctgtcgattttgcagatggggacaacctggggcctcccggaactctgccaccctggggaagttgggggagggccttagtcccggatcacaaccccgtctgctccccagaatcctttcctaagaatcgttgaggaccaaagttgtctttgctgacacgtgttgcttttctctttgccttttattgtttcagagaaaaatcaagttgactgtgtcaagtaacaccccaccccttacccccgtccagccatagtggctctctggagacacaggtcacaggcggagggtcccctgatcatccccaaccacacagccagggggacttgacccctgtccacccctgtctcgtgctccctcagacccccacaaaccggccaagcagtccggggaggcttcccctccacacaactcttagcatgtgattgcagatgtgaaatcaaaacgttgtttgttttttgttttgttttgattctaccccgtcggtccagtgtctgcacagacgccttcatttctctgtaaatatgtgacttggaacaaatgtttaacacaaacgagaagtggtcatgaatgcatggtgttgagatgttttgcactattctgactttttggtctctgtaaaaatattttattaacagcagacattaaaaaaagaaaaaccacacacagccttggacacgtggttgcctcctccttgcattcctttatcagcaaaaaaacaaaaacaaaaacctcacgtacgattagcacctactaggtaggcgcagactcagtagaacacactgcttgcccatgaacgcggctcatccgcaactggagtcatccccattttacagagaaggaaactgaagtacagagagacaagatcttgctcagatcacatagccaggaagagatggagctgggacttgaactcagaacatttgcaaccattgctttttttttttttttttgagatgaagtctctgttgcccaggctggaatgcagtggtgcaatctcagctcactgcaatctccacctcccagattcaagcgattctcctgccttagcctcctgagtagctgggattacaggcgcccgccaccttgcctggctaatttttgtatttttagtagagataggatttcaccatgttggccaggctggtcgcgaacccctgacctcaggtgatccactccctcggcctcccaaagtgctgggattacaggcgtgagccaccgcacccggccatctgttgcccttaaggaagttaagatacaaacactaggaatacatgcccttaattcaacccacacccccatgcttgtttggttttgttgggttttttgggatcttcttgtttgtttgtttttgagacagggtcttgctctgtcacccaggatggagttcagtgtcacaatctcaactcactgcatcctcaacctcccaggctcaggtgatcctcccacctcagcctcctgagtagtgtgcaccaccacacctggctaatttgtgtattttttgtagtgataggatgtcgccatgttgcccagactgctctcgaactcctcactttgggcaatcctctctcctcagccctccaaagtgttgggattacaggcgtgagccactgtgcctggccatgtctgttttaaatgtagcagctggggccaggcgcagtggctcacgcctgtaatcccagcactttaggaggccgaggcgggtggatcacgaggtcaggagatcgagaccatcctggctaacacggtgaaaccccgtctctactaaaagtaccaaaaattagccgggcacggtggcaggcacctgtagttccagctactcgggaggctgaggcaggagaatggtgtgaactggggaggcggagcttgcagtgagccaagatcacgccactgcattccagcctgggtgacggagcgagactccatctcaaaaaaaaaaaaagaaaaaaaagtagcagctgggaatctcatttcataaactcagatgtgtgaggaatgaaactccattttccaacagctgaatttaatttctgttttgagtctccatcccacgaggaagcagaaatggaatctcattcagcccagtttcctcggtaaattccgaaattcccctccttatgtctggggttatttctacatttgagcggacaggacgggagctgcgggatccaccaccaggagtgggttcagtgagtgcttttggttgcaagtaacagacagcaggactaaaagtggggccagaaagtggcttacaccaggagttatccagcacgcagcaatcccgctcctgggggacttttctcctttgagaaacttgcacgtgggggtgtggtatcttctcaaagaatagcatatagctgggaaaaggggacaaaccgcagctgtctgcatgatgtctgcagaggcagcctgcaggtccttgagtgcccctgctcattagctgtttctgtacccaagtgcataggctacgcactgtgactgatgtggatctatttgcccctcgctacaaaagggtctcaagtcttgtccttcagtgtgtgggacccatcgtgttaccaatggggcttggggactgaggctcaagactgcttgctgggaaatgtaacccctttgttctggacaggtagaaggagaggagaatggtcatccaggcctaatgtaaagtttctgcaacccaacccaattcccagactcattttggtcatgatcaatacaggggtcaacaaaattttctgtaaagggcgagctagtaaatagatttgctgtgtgtactatgctgtctctgttgcaaccaactcggccattgtaaccttgacaacatgcaaatgatgggcgtgactgcatgccaataaaactttatttagaaaatcaagcggatatattcaaacaaacccttggacgtgttcagaatagcgctattcacaatagccaaaaggtggaaacaactcaagtgtccaccaacaaatgaatggataaacacaacatgttgtctattcatacaatgaatatgattcagccataaaaaggaaggaagtggccgggcgcggtggctcacgcctgtaatcctagcactttgggaggctgaggcgagtggatcacctgaggtcaggagttcaagaccagcctggccaacatggtgaaaccctgtctgtactaaaaatacaaaattagccgggcatggtggcaagtgcttgtaatcccggctactcgggaggctgaggctggagaatcatttgaacctgggaggcagaggttacagtgagccgagatcctgccattgcactccagcctgggtgagaagagtgaaactccatctcaaaaaaaaaaaaaaaaaaaggaaggaaaccccgtctctactaaaaatacaaaaaattagccgggcatggtggtgggcgcctgtagtcccagctactcgggaggctgaggcaggagaatggcgtgaacccaggaggcagagcttgcagagagccgagatcgcgccactgcactgcagcctgggcgacagagcaagactccgtctcaacaacaacagcaataatagaagctggcccaaacccaccaaaaccaagatggcgacaagagtgacctctggtcatcctcactgctacactccccccatcaccatgacagtttacaaataccatggcaacatcaggaagttaccctatttggtctaaaaacgcaaggcatgaataatctacccttcctttagcatatcatcaagaaataaccataacaatgggcaaccagcagccttggggctgctctgtctatggagtagccattctttttttttttttttttttttgagatggagtcttgctctgtcacccaggccggagtgcagtggcgcggtttcggctcactgcaagctccacctcccgggtttcatgggcgtccatgtgaagaaaaaggagcatctatacaggagctcaaatgggctgtaccctgtagcattctgaggacaggtctgacttctgagaagggaaagtggtaaaagtat

SEQ ID NO: 2 - DNA, 9847 bp, Homo sapiens
atgtctctgctttgcgttggagtcaaaaaagccaagtttgatggtgcccaagagaaattcaacacgtacgtgaccctgaaagtgcagaatgtcaagagcacgaccatcgcggtgcggggcagccagcccagctgggagcaggatttcatgttcgagattaaccgtctggatttgggactgacggtggaggtgtggaataagggtctcatctgggacacaatggtgggcactgtgtggatcccactgaggaccatccgccagtccaatgaggagggccctggagagtggctgacgctggactcccaggtcatcatggcagacagtgagatctgtggcaccaaggaccccaccttccaccgcatcctcctggacacgcgctttgagctacccttagacattcctgaagaggaggctcgctactgggccaagaagctggagcagctcaatgctatgcgggaccaggatgaatattcgttccaagatgagcaagacaagcctctgcctgtccccagcaaccagtgctgcaactggaattattttggctggggtgagcagcacaacgatgaccccgacagtgcagtggatgatcgtgacagtgactaccgcagtgaaacgagcaacagcatcccgccgccctattatactacgtcacaacccaacgcctcagtccaccaatattctgttcgcccaccacccctgggctcccgggagtcctacagtgactccatgcacagttacgaggagttctctgagccacaagccctcagccccacgggtagcagccgctatgcctcttccggggagctgagccagggaagctctcagctgagcgaggacttcgaccctgacgagcacagcctgcagggctccgacatggaggatgagcgggaccgggactcctaccactcctgccacagctcggtcagctaccacaaagactcgcctcgctgggaccaggatgaggaagagctggaggaggacctggaggacttcctggaggaggaggagctgcctgaagatgaggaggagctggaggaggaggaggaggaggtgcctgacgatttgggcagctatgcccagcgtgaagacgtagctgtggctgagcccaaagacttcaaacgcatcagcctcccgccagctgccccagggaaggaggacaaggccccagtggcacccaccgaggcccccgacatggccaaggtggcccccaagccagccacgcccgacaaggtgcctgcagctgagcagatccctgaggctgagccacccaaggacgaggagagtttcaggccgagagaggatgaggaaggccaggaggggcaggactccatgtccagggccaaggccaactggctgcgtgccttcaacaaggtgcggatgcagctgcaggaggcccggggagaaggagagatgtctaaatccctatggttcaaaggcggcccagggggcggtctcatcatcatcgacagcatgccagacatccgcaagaggaaacctatcccactcgtgagcgacttggccatgtccctggtccagtccaggaaagcgggcatcacctcggccttggcctccagcacgttgaacaacgaggagctgaaaaaccacgtttacaagaagaccctgcaagccttaatctaccccatctcgtgcacgacgccacacaacttcgaagtgtggacggccaccacgcccacctactgctacgagtgcgaggggctgctgtggggcatcgcgaggcagggcatgcgctgcaccgagtgcggtgtcaagtgccacgagaagtgccaggacctgctcaacgccgactgcctgcagcgggctgcggagaagagctccaagcacggggcggaggaccggacacagaacatcatcatggtgctcaaggaccgcatgaagatccgggagcgcaacaagcccgagatcttcgagctcatccaggagatcttcgcggtgaccaagacggcgcacacgcagcagatgaaggcggtcaagcagagcgtgctggacggcacgtccaagtggtccgccaagatcagcatcaccgtggtctgcgcccagggcttgcaggcaaaggacaagacaggatccagtgacccctatgtcaccgtccaggtcgggaagaccaagaaacggacaaaaaccatctatgggaacctcaacccggtgtgggaggagaatttccactttgaatgtcacaattcctccgaccgcatcaaggtgcgcgtctgggacgaggatgacgacatcaaatcccgcgtgaaacagaggttcaagagggaatctgacgatttcctggggcagacgatcattgaggtgcggacgctcagcggcgagatggacgtgtggtacaacctggacaagcgaactgacaaatctgccgtgtcgggtgccatccggctccacatcagtgtggagatcaaaggcgaggagaaggtggccccgtaccatgtccagtacacctgtctgcatgagaacctgttccacttcgtgaccgacgtgcagaacaatggggtcgtgaagatcccagatgccaagggtgacgatgcctggaaggtttactacgatgagacagcccaggagattgtggacgagtttgccatgcgctacggcgtcgagtccatctaccaagccatgacccactttgcctgcctctcctccaagtatatgtgcccaggggtgcctgccgtcatgagcaccctgctcgccaacatcaatgcctactacgcacacaccaccgcctccaccaacgtgtctgcctccgaccgcttcgccgcctccaactttgggaaagagcgcttcgtgaaactcctggaccagctgcataactccctgcggattgacctctccatgtaccggaataacttcccagccagcagcccggagagactccaggacctcaaatccactgtggaccttctcaccagcatcaccttctttcggatgaaggtacaagaactccagagcccgccccgagccagccaggtggtaaaggactgtgtgaaagcctgccttaattctacctacgagtacatcttcaataactgccatgaactgtacagccgggagtaccagacagacccggccaagaagggggaagttctcccagaggaacaggggcccagcatcaagaacctcgacttctggtccaagctgattaccctcatagtgtccatcattgaggaagacaagaattcctacactccctgcctcaaccagtttccccaggagctgaatgtgggtaaaatcagcgctgaagtgatgtggaatctgtttgcccaagacatgaagtacgccatggaggagcacgacaagcatcgtctatgcaagagtgccgactacatgaacctccacttcaaggtgaaatggctctacaatgagtatgtgacggaacttcccgccttcaaggaccgcgtgcctgagtaccctgcatggtttgaacccttcgtcatccagtggctggatgagaatgaggaggtgtcccgggatttcctgcacggtgccctggagcgagacaagaaggatgggttccagcagacctcagagcatgccctattctcctgctccgtggtggatgttttctcccaactcaaccagagctttgaaatcatcaagaaactcgagtgtcccgaccctcagatcgtggggcactacatgaggcgctttgccaagaccatcagtaatgtgctcctccagtatgcagacatcatctccaaggactttgcctcctactgctccaaggagaaggagaaagtgccctgcattctcatgaataacactcaacagctacgagttcagctggagaagatgttcgaagccatgggaggaaaggagctggatgctgaagccagtgacatcctgaaggagcttcaggtgaaactcaataacgtcttggatgagctcagccgggtgtttgctaccagcttccagccgcacattgaagagtgtgtcaaacagatgggtgacatccttagccaggttaagggcacaggcaatgtgccagccagtgcctgcagcagcgtggcccaggacgcggacaatgtgttgcagcccatcatggacctgctggacagcaacctgaccctctttgccaaaatctgtgagaagactgtgctgaagcgagtgctgaaggagctgtggaagctggttatgaacaccatggagaaaaccatcgtcctgccgcccctcactgaccagacgatgatcgggaacctcttgagaaaacatggcaagggattagaaaagggcagggtgaaattgccaagccactcagacggaacccagatgatcttcaatgcagccaaggagctgggtcagctgtccaaactcaaggatcacatggtacgagaagaagccaagagcttgaccccaaagcagtgcgcggttgttgagttggccctggacaccatcaagcaatatttccacgcgggtggcgtgggcctcaagaagaccttcctggagaagagcccggacctgcaatccttgcgctatgccctgtcgctctacacgcaggccaccgacctgctaatcaagacctttgtacagacgcaatcggcccagggcttgggtgtagaagaccctgtgggtgaagtctctgtccatgttgagctgttcactcatccaggaactggggaacacaaggtcacagtgaaagtggtggctgccaatgacctcaagtggcagacttctggcatcttccggccgttcatcgaggtcaacatcattgggccccagctcagcgacaagaaacgcaagtttgcgaccaaatccaagaacaatagctgggctcccaagtacaatgagagcttccagttcacgctgagcgccgacgcgggtcccgagtgctatgagctgcaggtgtgcgtcaaggactactgcttcgcgcgcgaggaccgcacggtggggctggccgtgctgcagctgcgtgagctggcccagcgcgggagcgccgcctgctggctgccgctcggccgccgcatccacatggacgacacgggcctcacggtgctgcgaatcctctcgcagcgcagcaacgacgaggtggccaaggagttcgtgaagctcaagtcggacacgcgctccgccgaggagggcggtgccgcgcctgcgccttagcgcgggcggtcggccgagcggcactgcgcctgcgcggagggcgctgggcggggagggacggggcttgcgccttggtgggacctccccaggggcggggctcggggggctccacgccaagggtgggctgcgcctacgcccttgactcagctttcccttttggggaattaggaatggaggatgccccgccctctcgggaggccacgcccaagggcgcgacgaaggaaggagccacatccccaacttgaggccacgcccccagcacctagggggcattttgagctgggatgggggaaacctcgtccctatggaggaggccacatcccggggctctggtaccgggaggcaccacctcatgtcccctggaaaagccataagatgggacccagacccctgggaccccagaccaattgccaagtatggaaatctcagctccctcgagggggggccctgggcaaggggtagggctctctggagcgcccctctaggtggcctggggactggagggaccaggatgctggttggagggccccggaataccggagtccctttagatatttgtgcaaaaaataaatggggggaggggggaggatgggatttcaaaagcacatgcgcccttgggcgcccaaaccctgggggccgaggggacggctctggttccccacgctgcccctacttccctttgggagtttgcctctccctctcccccaacaaacccagtcctcatatcatagagttcaacacacccatttgacagatggcaaaactgaggcttaaagagctgcttgagacttggccaaggttccaggtgccataccctctgtgcccctcccttaggcctgtgtgccccatggaagggtgggctgagatcgggatgacctgacacagctccctattgctgctaattccccctcggcctcctccaaggggtgggaattccaggccaagacccctacttcgcctttccttctccggctgccaagcaggacctttgccctcagccctttctcctgggatctccatgggggatgccatgagggcctcccaccacaaaagagaatttgggatcccctggtcccaggtttctccatcccttcttccttttccagaattttccaaataggaaagaacagaaggagaccagaaactctaggggggagaaagagaatgagagaaagagaatgagagagagagaaacacaaacacagtgacacagtgagagcttagtctccaagagcctattcattgattcaaacacccaagccacaggatacctcagatggccctcttgccagctggaagctctttctccaatgagcaaagttacagtgacctggctggagttacctggtgcacataggaccttaggggaaagttcagcgtggactacacttgctctgggatctgcttttccacatgtgtgtatggcacgcctttttctgctggattgggaaggacaagattttgctgtgctagggagaaatgaaaacggggtgagctgagtagctgggtttctggaggatagaacatcagatggggaggctttccgaggtgaagaatgagagggaaccacttactagagagaaaagagctccaggcctggggaacagcacgtgcgaaggccaggagagaagaactgttgaaacaacgagaagggtggcacggctggagctgagccagcaagggggatcgtgaggagccttggggttggggagatctgcagaagcatcagaccaggcagggcctcgtacgcagtcctgaggagttttacttttattctaagacagttggggagctccaggagctgttttaagttggggagagactggattccagcctgcaaaagctgttttgtgaagactaaaaccagtgaggagaggtggaggtgctttggggacactgaaatggattcttggaaagattctgaaggctgtgttgaaaagacacctatagctgtggggacatgactataatcccagcatttggggagaccgaggctggcagatcacttaaggtcaggagtttgagaccagcctggccaacatggcgaaaccccatctctgctaaaaatacaaaaattagctgggtgcagtggtgcatgcctgtagtcccagctactcaggagactgaggcgggagaattgtttgaaccctggaggcagaggttgtagtgagtcgtgatcacacaactgcactccagcctgggcaacagaacaatactccattccctcccctctaccccaccaaaaaaaaaaaaaaatcctgcccttagatgagctctagggctgctgagtacagttgtcccagttgcacagtgcccaagggtttggcattgctaagaaggccacgtgcaaatcctagatattgagtgttgtatgtttgtgacgttggtttcccgacatgtgaatggcccaagtgtctggaagaagtggcgccactttctaatttgcttggagatgttgcatgtcccttaaattcagacaggtgcaggtaactggaggttctgaaccaaaggttaaaatgcaaattctcatacagggttgggaagttgtagccagggataagcttatgtgactgttatatggactgaggagcagatgtgaatttcgaaccatgacatggctgagggtaggggtcgggtggatggatgattcagggttgtaacccatagagcccaaaggggaagtgatctgtgacctggggtgagggtgatctggaagatttttggatggctggaaagaaatggggaagtcgagctgcctgagagagccaagttatttcccaaaagattccttaggagtctttctgttcaagacctccgtgtgtgtgtgtgtgtgtttagggttccccagcaatggcccaggcatgtgaaggaaacaagcttcttcagggaatatttgttgaatgagttttcctgactcccaggctagaactgtttttgcaatttccaccctcttttctttcccccagagaactcctattcgtccttcaaaacccatcacggaaacccctcttggagaaaaccctccttccttcccctcaggactttcccagccaccgtctctcctccagtccagcctgatgccatgggactgggggtttctctgtccagctctgtttctcccagactggggtctgaggactctcaggacccccaactttacctagcacaggctgggcacaagtgggtgacagggagtctacgcctagtggaattatgtattggggcagggtcagtgtgagaatacacatccgcatgcatgtctgtccatgtctgtccgtaccaaccttccccttccacacggacctgggcacataggaggtgtctgagcctgacacatgggacagagagtggacatggctgagacacggacagagaaaagacaaggagtccagggggctgaaagccttttgaaatcaggaagttcctgtattggcagaacaaagcccagagaggagcagggctttcctcaacgccacccagcaagtggacacagagcccggccttggatgacacctccagggttctgaaccctggacctcgctttatgcaaggagctggccccacatttccatgaatcggggaaacagcacaagaaggttggcctgtggcagggcaagggttaaaggggtgacattgagggatgcctcagagtcaaagtcccctgaccaagaggaatagagtagaaaacacagagacagagggtgagatcacgccccgatgaggacggagagagacagagatggagagagacatagaggtggaaatatacagagaaagataaatgcagagaccaaggcagggagtgtcgggggaagtaaagagggtgtcctgaagaaagaaggatctgttcactcttaccagtctgtcctcgaatgatttgcataaaatgaggaggtgcctgtccacacccccaattcctctctcaggccccagagcctgagacctcaccatgcccccatcagagatgcaaaaaactaaacacccaactagaaatccttgggacctctctcggctgggatctcagagcctttctgtcccctacccctaccccatgtgctgtcgattttgcagatggggacaacctggggcctcccggaactctgccaccctggggaagttgggggagggccttagtcccggatcacaaccccgtctgctccccagaatcctttcctaagaatcgttgaggaccaaagttgtctttgctgacacgtgttgcttttctctttgccttttattgtttcagagaaaaatcaagttgactgtgtcaagtaacaccccaccccttacccccgtccagccatagtggctctctggagacacaggtcacaggcggagggtcccctgatcatccccaaccacacagccagggggacttgacccctgtccacccctgtctcgtgctccctcagacccccacaaaccggccaagcagtccggggaggcttcccctccacacaactcttagcatgtgattgcagatgtgaaatcaaaacgttgtttgttttttgttttgttttgattctaccccgtcggtccagtgtctgcacagacgccttcatttctctgtaaatatgtgacttggaacaaatgtttaacacaaacgagaagtggtcatgaatgcatggtgttgagatgttttgcactattctgactttttggtctctgtaaaaatattttattaacagcagacattaaaaaaagaaaaaccacacacagccttggaaaaaaaaaa

SEQ ID NO: 3 - DNA, 128 bp, Homo sapiens
ctgcctgggtttcctggaaagaactcttatccccaggaactagtttgttgaataaatgctggtgaatgaatgaatgattgaacagatgaatgagtgatgagtagataaaaggatggatggagagatgg

SEQ ID NO: 4 - DNA, 178 bp, Homo sapiens
ccctaaccactcaggattgggccgtttgtgtctgggtatgtctcttccagctgcctgggtttcctggaaagaactcttatccccaggaactagtttgttgaataaatgctggtgaatgaatgaatgattgaacagatgaatgagtgatgagtagataaaaggatggatggagagatgg

SEQ ID NO: 5 - DNA, 431 bp, Homo sapiens
gtgagggtcattgctcggcccctcccatgccacttccactcaccattcctgcctgcccagctcttcctctttctggccacaccatccacactctcctggccctctgagactgcccgccatgccattccctttacctggaaaactcctccctatccatcaaagtccagattcagggtcacctcctctgggaagcccaccttggcctccaggttgactctcactactcatcatcaggttcttccttctattccagccctaaccactcaggattgggccgtttgtgtctgggtatgtctcttccagctgcctgggtttcctggaaagaactcttatccccaggaactagtttgttgaataaatgctggtgaatgaatgaatgattgaacagatgaatgagtgatgagtagataaaaggatggatggagagatgg

SEQ ID NO: 6 - DNA, 22 bp, Artificial sequence
  Primer
gtacaacctggacaagcgaact

SEQ ID NO: 7 - DNA, 19 bp, Artificial sequence
  Primer
ggaaacccaggcagctcat

SEQ ID NO: 8 - DNA, 23 bp, Artificial sequence
  Probe
atcaaaggcgaggagaaggtggc

SEQ ID NO: 9 - DNA, 20 bp, Artificial sequence
  Primer
tggatggagagatggaacct

SEQ ID NO: 10 - DNA, 22 bp, Artificial sequence
  Primer
gggctgtctcatcgtagtaaac

SEQ ID NO: 11 - DNA, 24 bp, Artificial sequence
  Probe
ttggcatctgggatcttcacgacc

SEQ ID NO: 12 - DNA, 21 bp, Artificial sequence
  Primer
cacctgtctgcatgagaacct

SEQ ID NO: 13 - DNA, 22 bp, Artificial sequence
  Primer
catggcaaactcgtccacaatc

SEQ ID NO: 14 - DNA, 24 bp, Artificial sequence
  Probe
taaaccttccaggcatcgtcaccc

SEQ ID NO: 15-20 - skipped (6 entries)

SEQ ID NO: 21 - DNA, 18 bp, Artificial sequence
  Synthetic oligonucleotide
tagttcctggggataaga

SEQ ID NO: 22 - DNA, 18 bp, Artificial sequence
  Synthetic oligonucleotide
agttcctggggataagag

SEQ ID NO: 23 - DNA, 18 bp, Artificial sequence
  Synthetic oligonucleotide
gttcctggggataagagt

SEQ ID NO: 24 - DNA, 18 bp, Artificial sequence
  Synthetic oligonucleotide
ttcctggggataagagtt

SEQ ID NO: 25 - DNA, 18 bp, Artificial sequence
  Synthetic oligonucleotide
tcctggggataagagttc

SEQ ID NO: 26 - DNA, 18 bp, Artificial sequence
  Synthetic oligonucleotide
cctggggataagagttct

SEQ ID NO: 27 - DNA, 18 bp, Artificial sequence
  Synthetic oligonucleotide
ctggggataagagttctt

SEQ ID NO: 28 - DNA, 18 bp, Artificial sequence
  Synthetic oligonucleotide
tggggataagagttcttt

SEQ ID NO: 29 - DNA, 18 bp, Artificial sequence
  Synthetic oligonucleotide
ggggataagagttctttc

SEQ ID NO: 30 - DNA, 18 bp, Artificial sequence
  Synthetic oligonucleotide
gggataagagttctttcc

SEQ ID NO: 31 - DNA, 18 bp, Artificial sequence
  Synthetic oligonucleotide
agccacgaatctacccac

SEQ ID NO: 32 - DNA, 18 bp, Artificial sequence
  Synthetic oligonucleotide
catctgttcaatcattca

SEQ ID NO: 33 - DNA, 18 bp, Artificial sequence
  Synthetic oligonucleotide
tctgttcaatcattcatt

SEQ ID NO: 34 - DNA, 18 bp, Artificial sequence
  Synthetic oligonucleotide
tgttcaatcattcattca

SEQ ID NO: 35 - DNA, 18 bp, Artificial sequence
  Synthetic oligonucleotide
ttcaatcattcattcatt

SEQ ID NO: 36 - DNA, 18 bp, Artificial sequence
  Synthetic oligonucleotide
caatcattcattcattca

SEQ ID NO: 37 - DNA, 18 bp, Artificial sequence
  Synthetic oligonucleotide
atcattcattcattcacc

SEQ ID NO: 38 - DNA, 18 bp, Artificial sequence
  Synthetic oligonucleotide
cattcattcattcaccag

SEQ ID NO: 39 - DNA, 18 bp, Artificial sequence
  Synthetic oligonucleotide
ttcattcattcaccagca

SEQ ID NO: 40 - DNA, 18 bp, Artificial sequence
  Synthetic oligonucleotide
cattcattcaccagcatt

SEQ ID NO: 41 - DNA, 18 bp, Artificial sequence
  Synthetic oligonucleotide
ttcattcaccagcattta

SEQ ID NO: 42 - DNA, 18 bp, Artificial sequence
  Synthetic oligonucleotide
ccacgaatctacccacca

SEQ ID NO: 43 - DNA, 18 bp, Artificial sequence
  Synthetic oligonucleotide
cattcaccagcatttatt

SEQ ID NO: 44 - DNA, 18 bp, Artificial sequence
  Synthetic oligonucleotide
ttcaccagcatttattca

SEQ ID NO: 45 - DNA, 18 bp, Artificial sequence
  Synthetic oligonucleotide
caccagcatttattcaac

SEQ ID NO: 46 - DNA, 18 bp, Artificial sequence
  Synthetic oligonucleotide
ccagcatttattcaacaa

SEQ ID NO: 47 - DNA, 18 bp, Artificial sequence
  Synthetic oligonucleotide
agcatttattcaacaaac

SEQ ID NO: 48 - DNA, 18 bp, Artificial sequence
  Synthetic oligonucleotide
catttattcaacaaacta

SEQ ID NO: 49 - DNA, 18 bp, Artificial sequence
  Synthetic oligonucleotide
tttattcaacaaactagt

SEQ ID NO: 50 - DNA, 18 bp, Artificial sequence
  Synthetic oligonucleotide
tattcaacaaactagttc

SEQ ID NO: 51 - DNA, 18 bp, Artificial sequence
  Synthetic oligonucleotide
ttcaacaaactagttcct

SEQ ID NO: 52 - DNA, 18 bp, Artificial sequence
  Synthetic oligonucleotide
caacaaactagttcctgg

SEQ ID NO: 53 - DNA, 18 bp, Artificial sequence
  Synthetic oligonucleotide
acgaatctacccaccaac

SEQ ID NO: 54 - DNA, 18 bp, Artificial sequence
  Synthetic oligonucleotide
acaaactagttcctgggg

SEQ ID NO: 55 - DNA, 18 bp, Artificial sequence
  Synthetic oligonucleotide
aaactagttcctggggat

SEQ ID NO: 56 - DNA, 18 bp, Artificial sequence
  Synthetic oligonucleotide
actagttcctggggataa

SEQ ID NO: 57 - DNA, 18 bp, Artificial sequence
  Synthetic oligonucleotide
ggataagagttctttcca

SEQ ID NO: 58 - DNA, 18 bp, Artificial sequence
  Synthetic oligonucleotide
gataagagttctttccag

SEQ ID NO: 59 - DNA, 18 bp, Artificial sequence
  Synthetic oligonucleotide
ataagagttctttccagg

SEQ ID NO: 60 - DNA, 18 bp, Artificial sequence
  Synthetic oligonucleotide
taagagttctttccagga

SEQ ID NO: 61 - DNA, 18 bp, Artificial sequence
  Synthetic oligonucleotide
gaatctacccaccaactc

SEQ ID NO: 62 - DNA, 18 bp, Artificial sequence
  Synthetic oligonucleotide
aagagttctttccaggaa

SEQ ID NO: 63 - DNA, 18 bp, Artificial sequence
  Synthetic oligonucleotide
agagttctttccaggaaa

SEQ ID NO: 64 - DNA, 18 bp, Artificial sequence
  Synthetic oligonucleotide
gagttctttccaggaaac

SEQ ID NO: 65 - DNA, 18 bp, Artificial sequence
  Synthetic oligonucleotide
agttctttccaggaaacc

SEQ ID NO: 66 - DNA, 18 bp, Artificial sequence
  Synthetic oligonucleotide
gttctttccaggaaaccc

SEQ ID NO: 67 - DNA, 18 bp, Artificial sequence
  Synthetic oligonucleotide
ttctttccaggaaaccca

SEQ ID NO: 68 - DNA, 18 bp, Artificial sequence
  Synthetic oligonucleotide
tctttccaggaaacccag

SEQ ID NO: 69 - DNA, 18 bp, Artificial sequence
  Synthetic oligonucleotide
ctttccaggaaacccagg

SEQ ID NO: 70 - DNA, 18 bp, Artificial sequence
  Synthetic oligonucleotide
tttccaggaaacccaggc

SEQ ID NO: 71 - DNA, 18 bp, Artificial sequence
  Synthetic oligonucleotide
ttccaggaaacccaggca

SEQ ID NO: 72 - DNA, 18 bp, Artificial sequence
  Synthetic oligonucleotide
tccaggaaacccaggcag

SEQ ID NO: 73 - DNA, 18 bp, Artificial sequence
  Synthetic oligonucleotide
ccaggaaacccaggcagc

SEQ ID NO: 74 - DNA, 18 bp, Artificial sequence
  Synthetic oligonucleotide
caggaaacccaggcagct

SEQ ID NO: 75 - DNA, 18 bp, Artificial sequence
  Synthetic oligonucleotide
aggaaacccaggcagctg

SEQ ID NO: 76 - DNA, 18 bp, Artificial sequence
  Synthetic oligonucleotide
ggaaacccaggcagctgg

SEQ ID NO: 77 - DNA, 18 bp, Artificial sequence
  Synthetic oligonucleotide
gaaacccaggcagctgga

SEQ ID NO: 78 - DNA, 18 bp, Artificial sequence
  Synthetic oligonucleotide
aaacccaggcagctggaa

SEQ ID NO: 79 - DNA, 18 bp, Artificial sequence
  Synthetic oligonucleotide
aacccaggcagctggaag

SEQ ID NO: 80 - DNA, 18 bp, Artificial sequence
  Synthetic oligonucleotide
acccaggcagctggaaga

SEQ ID NO: 81 - DNA, 18 bp, Artificial sequence
  Synthetic oligonucleotide
cccaggcagctggaagag

SEQ ID NO: 82 - DNA, 18 bp, Artificial sequence
  Synthetic oligonucleotide
atctacccaccaactcat

SEQ ID NO: 83 - DNA, 18 bp, Artificial sequence
  Synthetic oligonucleotide
ccaggcagctggaagaga

SEQ ID NO: 84 - DNA, 18 bp, Artificial sequence
  Synthetic oligonucleotide
caggcagctggaagagac

SEQ ID NO: 85 - DNA, 18 bp, Artificial sequence
  Synthetic oligonucleotide
aggcagctggaagagaca

SEQ ID NO: 86 - DNA, 18 bp, Artificial sequence
  Synthetic oligonucleotide
ggcagctggaagagacat

SEQ ID NO: 87 - DNA, 18 bp, Artificial sequence
  Synthetic oligonucleotide
gcagctggaagagacata

SEQ ID NO: 88 - DNA, 18 bp, Artificial sequence
  Synthetic oligonucleotide
cagctggaagagacatac

SEQ ID NO: 89 - DNA, 18 bp, Artificial sequence
  Synthetic oligonucleotide
agctggaagagacatacc

SEQ ID NO: 90 - DNA, 18 bp, Artificial sequence
  Synthetic oligonucleotide
gctggaagagacataccc

SEQ ID NO: 91 - DNA, 18 bp, Artificial sequence
  Synthetic oligonucleotide
ctggaagagacataccca

SEQ ID NO: 92 - DNA, 18 bp, Artificial sequence
  Synthetic oligonucleotide
tggaagagacatacccag

SEQ ID NO: 93 - DNA, 18 bp, Artificial sequence
  Synthetic oligonucleotide
ggaagagacatacccaga

SEQ ID NO: 94 - DNA, 18 bp, Artificial sequence
  Synthetic oligonucleotide
gaagagacatacccagac

SEQ ID NO: 95 - DNA, 18 bp, Artificial sequence
  Synthetic oligonucleotide
aagagacatacccagaca

SEQ ID NO: 96 - DNA, 18 bp, Artificial sequence
  Synthetic oligonucleotide
agagacatacccagacac

SEQ ID NO: 97 - DNA, 18 bp, Artificial sequence
  Synthetic oligonucleotide
gagacatacccagacaca

SEQ ID NO: 98 - DNA, 18 bp, Artificial sequence
  Synthetic oligonucleotide
agacatacccagacacaa

SEQ ID NO: 99 - DNA, 18 bp, Artificial sequence
  Synthetic oligonucleotide
gacatacccagacacaaa

SEQ ID NO: 100 - DNA, 18 bp, Artificial sequence
  Synthetic oligonucleotide
acatacccagacacaaac

SEQ ID NO: 101 - DNA, 18 bp, Artificial sequence
  Synthetic oligonucleotide
catacccagacacaaacg

SEQ ID NO: 102 - DNA, 18 bp, Artificial sequence
  Synthetic oligonucleotide
atacccagacacaaacgg

SEQ ID NO: 103 - DNA, 18 bp, Artificial sequence
  Synthetic oligonucleotide
ctacccaccaactcatcc

SEQ ID NO: 104 - DNA, 18 bp, Artificial sequence
  Synthetic oligonucleotide
tacccagacacaaacggc

SEQ ID NO: 105 - DNA, 18 bp, Artificial sequence
  Synthetic oligonucleotide
acccagacacaaacggcc

SEQ ID NO: 106 - DNA, 18 bp, Artificial sequence
  Synthetic oligonucleotide
cccagacacaaacggccc

SEQ ID NO: 107 - DNA, 18 bp, Artificial sequence
  Synthetic oligonucleotide
ccagacacaaacggccca

SEQ ID NO: 108 - DNA, 18 bp, Artificial sequence
  Synthetic oligonucleotide
cagacacaaacggcccaa

SEQ ID NO: 109 - DNA, 18 bp, Artificial sequence
  Synthetic oligonucleotide
agacacaaacggcccaat

SEQ ID NO: 110 - DNA, 18 bp, Artificial sequence
  Synthetic oligonucleotide
gacacaaacggcccaatc

SEQ ID NO: 111 - DNA, 18 bp, Artificial sequence
  Synthetic oligonucleotide
acacaaacggcccaatcc

SEQ ID NO: 112 - DNA, 18 bp, Artificial sequence
  Synthetic oligonucleotide
cacaaacggcccaatcct

SEQ ID NO: 113 - DNA, 18 bp, Artificial sequence
  Synthetic oligonucleotide
acaaacggcccaatcctg

SEQ ID NO: 114 - DNA, 18 bp, Artificial sequence
  Synthetic oligonucleotide
caaacggcccaatcctga

SEQ ID NO: 115 - DNA, 18 bp, Artificial sequence
  Synthetic oligonucleotide
aaacggcccaatcctgag

SEQ ID NO: 116 - DNA, 18 bp, Artificial sequence
  Synthetic oligonucleotide
aacggcccaatcctgagt

SEQ ID NO: 117 - DNA, 18 bp, Artificial sequence
  Synthetic oligonucleotide
acggcccaatcctgagtg

SEQ ID NO: 118 - DNA, 18 bp, Artificial sequence
  Synthetic oligonucleotide
cggcccaatcctgagtgg

SEQ ID NO: 119 - DNA, 18 bp, Artificial sequence
  Synthetic oligonucleotide
ggcccaatcctgagtggt

SEQ ID NO: 120 - DNA, 18 bp, Artificial sequence
  Synthetic oligonucleotide
gcccaatcctgagtggtt

SEQ ID NO: 121 - DNA, 18 bp, Artificial sequence
  Synthetic oligonucleotide
cccaatcctgagtggtta

SEQ ID NO: 122 - DNA, 18 bp, Artificial sequence
  Synthetic oligonucleotide
ccaatcctgagtggttag

SEQ ID NO: 123 - DNA, 18 bp, Artificial sequence
  Synthetic oligonucleotide
caatcctgagtggttagg

SEQ ID NO: 124 - DNA, 18 bp, Artificial sequence
  Synthetic oligonucleotide
acccaccaactcatccat

SEQ ID NO: 125 - DNA, 18 bp, Artificial sequence
  Synthetic oligonucleotide
aatcctgagtggttaggg

SEQ ID NO: 126 - DNA, 18 bp, Artificial sequence
  Synthetic oligonucleotide
atcctgagtggttagggc

SEQ ID NO: 127 - DNA, 18 bp, Artificial sequence
  Synthetic oligonucleotide
tcctgagtggttagggct

SEQ ID NO: 128 - DNA, 18 bp, Artificial sequence
  Synthetic oligonucleotide
cctgagtggttagggctg

SEQ ID NO: 129 - DNA, 18 bp, Artificial sequence
  Synthetic oligonucleotide
ctgagtggttagggctgg

SEQ ID NO: 130 - DNA, 18 bp, Artificial sequence
  Synthetic oligonucleotide
tgagtggttagggctgga

SEQ ID NO: 131 - DNA, 18 bp, Artificial sequence
  Synthetic oligonucleotide
gagtggttagggctggaa

SEQ ID NO: 132 - DNA, 18 bp, Artificial sequence
  Synthetic oligonucleotide
agtggttagggctggaat

SEQ ID NO: 133 - DNA, 18 bp, Artificial sequence
  Synthetic oligonucleotide
gtggttagggctggaata

SEQ ID NO: 134 - DNA, 18 bp, Artificial sequence
  Synthetic oligonucleotide
tggttagggctggaatag

SEQ ID NO: 135 - DNA, 18 bp, Artificial sequence
  Synthetic oligonucleotide
ggttagggctggaataga

SEQ ID NO: 136 - DNA, 18 bp, Artificial sequence
  Synthetic oligonucleotide
gttagggctggaatagaa

SEQ ID NO: 137 - DNA, 18 bp, Artificial sequence
  Synthetic oligonucleotide
ttagggctggaatagaag

SEQ ID NO: 138 - DNA, 18 bp, Artificial sequence
  Synthetic oligonucleotide
tagggctggaatagaagg

SEQ ID NO: 139 - DNA, 18 bp, Artificial sequence
  Synthetic oligonucleotide
agggctggaatagaagga

SEQ ID NO: 140 - DNA, 18 bp, Artificial sequence
  Synthetic oligonucleotide
gggctggaatagaaggaa

SEQ ID NO: 141 - DNA, 18 bp, Artificial sequence
  Synthetic oligonucleotide
ggctggaatagaaggaag

SEQ ID NO: 142 - DNA, 18 bp, Artificial sequence
  Synthetic oligonucleotide
gctggaatagaaggaaga

SEQ ID NO: 143 - DNA, 18 bp, Artificial sequence
  Synthetic oligonucleotide
ctggaatagaaggaagaa

SEQ ID NO: 144 - DNA, 18 bp, Artificial sequence
  Synthetic oligonucleotide
tggaatagaaggaagaac

SEQ ID NO: 145 - DNA, 18 bp, Artificial sequence
  Synthetic oligonucleotide
ccaccaactcatccatct

SEQ ID NO: 146 - DNA, 18 bp, Artificial sequence
  Synthetic oligonucleotide
ggaatagaaggaagaacc

SEQ ID NO: 147 - DNA, 18 bp, Artificial sequence
  Synthetic oligonucleotide
gaatagaaggaagaacct

SEQ ID NO: 148 - DNA, 18 bp, Artificial sequence
  Synthetic oligonucleotide
aatagaaggaagaacctg

SEQ ID NO: 149 - DNA, 18 bp, Artificial sequence
  Synthetic oligonucleotide
atagaaggaagaacctga

SEQ ID NO: 150 - DNA, 18 bp, Artificial sequence
  Synthetic oligonucleotide
tagaaggaagaacctgat

SEQ ID NO: 151 - DNA, 18 bp, Artificial sequence
  Synthetic oligonucleotide
agaaggaagaacctgatg

SEQ ID NO: 152 - DNA, 18 bp, Artificial sequence
  Synthetic oligonucleotide
gaaggaagaacctgatga

SEQ ID NO: 153 - DNA, 18 bp, Artificial sequence
  Synthetic oligonucleotide
aggaagaacctgatgatg

SEQ ID NO: 154 - DNA, 18 bp, Artificial sequence
  Synthetic oligonucleotide
gaagaacctgatgatgag

SEQ ID NO: 155 - DNA, 18 bp, Artificial sequence
  Synthetic oligonucleotide
agaacctgatgatgagta

SEQ ID NO: 156 - DNA, 18 bp, Artificial sequence
  Synthetic oligonucleotide
aacctgatgatgagtagt

SEQ ID NO: 157 - DNA, 18 bp, Artificial sequence
  Synthetic oligonucleotide
cctgatgatgagtagtga

SEQ ID NO: 158 - DNA, 18 bp, Artificial sequence
  Synthetic oligonucleotide
tgatgatgagtagtgaga

SEQ ID NO: 159 - DNA, 18 bp, Artificial sequence
  Synthetic oligonucleotide
accaactcatccatctat

SEQ ID NO: 160 - DNA, 18 bp, Artificial sequence
  Synthetic oligonucleotide
atgatgagtagtgagagt

SEQ ID NO: 161 - DNA, 18 bp, Artificial sequence
  Synthetic oligonucleotide
gatgagtagtgagagtca

SEQ ID NO: 162 - DNA, 18 bp, Artificial sequence
  Synthetic oligonucleotide
tgagtagtgagagtcaac

SEQ ID NO: 163 - DNA, 18 bp, Artificial sequence
  Synthetic oligonucleotide
agtagtgagagtcaacct

SEQ ID NO: 164 - DNA, 18 bp, Artificial sequence
  Synthetic oligonucleotide
tagtgagagtcaacctgg

SEQ ID NO: 165 - DNA, 18 bp, Artificial sequence
  Synthetic oligonucleotide
gtgagagtcaacctggag

SEQ ID NO: 166 - DNA, 18 bp, Artificial sequence
  Synthetic oligonucleotide
gagagtcaacctggaggc

SEQ ID NO: 167 - DNA, 18 bp, Artificial sequence
  Synthetic oligonucleotide
gagtcaacctggaggcca

SEQ ID NO: 168 - DNA, 18 bp, Artificial sequence
  Synthetic oligonucleotide
gtcaacctggaggccaag

SEQ ID NO: 169 - DNA, 18 bp, Artificial sequence
  Synthetic oligonucleotide
caacctggaggccaaggt

SEQ ID NO: 170 - DNA, 18 bp, Artificial sequence
  Synthetic oligonucleotide
caactcatccatctatcc

SEQ ID NO: 171 - DNA, 18 bp, Artificial sequence
  Synthetic oligonucleotide
acctggaggccaaggtgg

SEQ ID NO: 172 - DNA, 18 bp, Artificial sequence
  Synthetic oligonucleotide
ctggaggccaaggtgggc

SEQ ID NO: 173 - DNA, 18 bp, Artificial sequence
  Synthetic oligonucleotide
ggaggccaaggtgggctt

SEQ ID NO: 174 - DNA, 18 bp, Artificial sequence
  Synthetic oligonucleotide
aggccaaggtgggcttcc

SEQ ID NO: 175 - DNA, 18 bp, Artificial sequence
  Synthetic oligonucleotide
gccaaggtgggcttccca

SEQ ID NO: 176 - DNA, 18 bp, Artificial sequence
  Synthetic oligonucleotide
caaggtgggcttcccaga

SEQ ID NO: 177 - DNA, 18 bp, Artificial sequence
  Synthetic oligonucleotide
aggtgggcttcccagagg

SEQ ID NO: 178 - DNA, 18 bp, Artificial sequence
  Synthetic oligonucleotide
gtgggcttcccagaggag

SEQ ID NO: 179 - DNA, 18 bp, Artificial sequence
  Synthetic oligonucleotide
gggcttcccagaggaggt

SEQ ID NO: 180 - DNA, 18 bp, Artificial sequence
  Synthetic oligonucleotide
gcttcccagaggaggtga

SEQ ID NO: 181 - DNA, 18 bp, Artificial sequence
  Synthetic oligonucleotide
actcatccatctatccat

SEQ ID NO: 182 - DNA, 18 bp, Artificial sequence
  Synthetic oligonucleotide
ttcccagaggaggtgacc

SEQ ID NO: 183 - DNA, 18 bp, Artificial sequence
  Synthetic oligonucleotide
cccagaggaggtgaccct

SEQ ID NO: 184 - DNA, 18 bp, Artificial sequence
  Synthetic oligonucleotide
cagaggaggtgaccctga

SEQ ID NO: 185 - DNA, 18 bp, Artificial sequence
  Synthetic oligonucleotide
gaggaggtgaccctgaat

SEQ ID NO: 186 - DNA, 18 bp, Artificial sequence
  Synthetic oligonucleotide
ggaggtgaccctgaatct

SEQ ID NO: 187 - DNA, 18 bp, Artificial sequence
  Synthetic oligonucleotide
aggtgaccctgaatctgg

SEQ ID NO: 188 - DNA, 18 bp, Artificial sequence
  Synthetic oligonucleotide
gtgaccctgaatctggac

SEQ ID NO: 189 - DNA, 18 bp, Artificial sequence
  Synthetic oligonucleotide
gaccctgaatctggactt

SEQ ID NO: 190 - DNA, 18 bp, Artificial sequence
  Synthetic oligonucleotide
ccctgaatctggactttg

SEQ ID NO: 191 - DNA, 18 bp, Artificial sequence
  Synthetic oligonucleotide
ctgaatctggactttgat

SEQ ID NO: 192 - DNA, 18 bp, Artificial sequence
  Synthetic oligonucleotide
tcatccatctatccatcc

SEQ ID NO: 193 - DNA, 18 bp, Artificial sequence
  Synthetic oligonucleotide
gaatctggactttgatgg

SEQ ID NO: 194 - DNA, 18 bp, Artificial sequence
  Synthetic oligonucleotide
atctggactttgatggat

SEQ ID NO: 195 - DNA, 18 bp, Artificial sequence
  Synthetic oligonucleotide
ctggactttgatggatag

SEQ ID NO: 196 - DNA, 18 bp, Artificial sequence
  Synthetic oligonucleotide
ggactttgatggataggg

SEQ ID NO: 197 - DNA, 18 bp, Artificial sequence
  Synthetic oligonucleotide
actttgatggatagggag

SEQ ID NO: 198 - DNA, 18 bp, Artificial sequence
  Synthetic oligonucleotide
tttgatggatagggagga

SEQ ID NO: 199 - DNA, 18 bp, Artificial sequence
  Synthetic oligonucleotide
tgatggatagggaggagt

SEQ ID NO: 200 - DNA, 18 bp, Artificial sequence
  Synthetic oligonucleotide
atggatagggaggagttt

SEQ ID NO: 201 - DNA, 18 bp, Artificial sequence
  Synthetic oligonucleotide
ggatagggaggagttttc

SEQ ID NO: 202 - DNA, 18 bp, Artificial sequence
  Synthetic oligonucleotide
atagggaggagttttcca

SEQ ID NO: 203 - DNA, 18 bp, Artificial sequence
  Synthetic oligonucleotide
atccatctatccatccat

SEQ ID NO: 204 - DNA, 18 bp, Artificial sequence
  Synthetic oligonucleotide
agggaggagttttccagg

SEQ ID NO: 205 - DNA, 18 bp, Artificial sequence
  Synthetic oligonucleotide
ggaggagttttccaggta

SEQ ID NO: 206 - DNA, 18 bp, Artificial sequence
  Synthetic oligonucleotide
aggagttttccaggtaaa

SEQ ID NO: 207 - DNA, 18 bp, Artificial sequence
  Synthetic oligonucleotide
gagttttccaggtaaagg

SEQ ID NO: 208 - DNA, 18 bp, Artificial sequence
  Synthetic oligonucleotide
gttttccaggtaaaggga

SEQ ID NO: 209 - DNA, 18 bp, Artificial sequence
  Synthetic oligonucleotide
tttccaggtaaagggaat

SEQ ID NO: 210 - DNA, 18 bp, Artificial sequence
  Synthetic oligonucleotide
tccaggtaaagggaatgg

SEQ ID NO: 211 - DNA, 18 bp, Artificial sequence
  Synthetic oligonucleotide
caggtaaagggaatggca

SEQ ID NO: 212 - DNA, 18 bp, Artificial sequence
  Synthetic oligonucleotide
ggtaaagggaatggcatg

SEQ ID NO: 213 - DNA, 18 bp, Artificial sequence
  Synthetic oligonucleotide
taaagggaatggcatggc

SEQ ID NO: 214 - DNA, 18 bp, Artificial sequence
  Synthetic oligonucleotide
ccatctatccatccatgt

SEQ ID NO: 215 - DNA, 18 bp, Artificial sequence
  Synthetic oligonucleotide
aagggaatggcatggcgg

SEQ ID NO: 216 - DNA, 18 bp, Artificial sequence
  Synthetic oligonucleotide
gggaatggcatggcgggc

SEQ ID NO: 217 - DNA, 18 bp, Artificial sequence
  Synthetic oligonucleotide
gaatggcatggcgggcag

SEQ ID NO: 218 - DNA, 18 bp, Artificial sequence
  Synthetic oligonucleotide
atggcatggcgggcagtc

SEQ ID NO: 219 - DNA, 18 bp, Artificial sequence
  Synthetic oligonucleotide
ggcatggcgggcagtctc

SEQ ID NO: 220 - DNA, 18 bp, Artificial sequence
  Synthetic oligonucleotide
catggcgggcagtctcag

SEQ ID NO: 221 - DNA, 18 bp, Artificial sequence
  Synthetic oligonucleotide
tggcgggcagtctcagag

SEQ ID NO: 222 - DNA, 18 bp, Artificial sequence
  Synthetic oligonucleotide
gcgggcagtctcagaggg

SEQ ID NO: 223 - DNA, 18 bp, Artificial sequence
  Synthetic oligonucleotide
gggcagtctcagagggcc

SEQ ID NO: 224 - DNA, 18 bp, Artificial sequence
  Synthetic oligonucleotide
gcagtctcagagggccag

SEQ ID NO: 225 - DNA, 18 bp, Artificial sequence
  Synthetic oligonucleotide
atctatccatccatgtac

SEQ ID NO: 226 - DNA, 18 bp, Artificial sequence
  Synthetic oligonucleotide
agtctcagagggccagga

SEQ ID NO: 227 - DNA, 18 bp, Artificial sequence
  Synthetic oligonucleotide
tctcagagggccaggaga

SEQ ID NO: 228 - DNA, 18 bp, Artificial sequence
  Synthetic oligonucleotide
tcagagggccaggagagt

SEQ ID NO: 229 - DNA, 18 bp, Artificial sequence
  Synthetic oligonucleotide
agagggccaggagagtgt

SEQ ID NO: 230 - DNA, 18 bp, Artificial sequence
  Synthetic oligonucleotide
agggccaggagagtgtgg

SEQ ID NO: 231 - DNA, 18 bp, Artificial sequence
  Synthetic oligonucleotide
ggccaggagagtgtggat

SEQ ID NO: 232 - DNA, 18 bp, Artificial sequence
  Synthetic oligonucleotide
ccaggagagtgtggatgg

SEQ ID NO: 233 - DNA, 18 bp, Artificial sequence
  Synthetic oligonucleotide
aggagagtgtggatggtg

SEQ ID NO: 234 - DNA, 18 bp, Artificial sequence
  Synthetic oligonucleotide
gagagtgtggatggtgtg

SEQ ID NO: 235 - DNA, 18 bp, Artificial sequence
  Synthetic oligonucleotide
gagtgtggatggtgtggc

SEQ ID NO: 236 - DNA, 18 bp, Artificial sequence
  Synthetic oligonucleotide
ccatctagccacgaatct

SEQ ID NO: 237 - DNA, 18 bp, Artificial sequence
  Synthetic oligonucleotide
ctatccatccatgtactc

SEQ ID NO: 238 - DNA, 18 bp, Artificial sequence
  Synthetic oligonucleotide
gtgtggatggtgtggcca

SEQ ID NO: 239 - DNA, 18 bp, Artificial sequence
  Synthetic oligonucleotide
gtggatggtgtggccaga

SEQ ID NO: 240 - DNA, 18 bp, Artificial sequence
  Synthetic oligonucleotide
ggatggtgtggccagaaa

SEQ ID NO: 241 - DNA, 18 bp, Artificial sequence
  Synthetic oligonucleotide
atggtgtggccagaaaga

SEQ ID NO: 242 - DNA, 18 bp, Artificial sequence
  Synthetic oligonucleotide
ggtgtggccagaaagagg

SEQ ID NO: 243 - DNA, 18 bp, Artificial sequence
  Synthetic oligonucleotide
tgtggccagaaagaggaa

SEQ ID NO: 244 - DNA, 18 bp, Artificial sequence
  Synthetic oligonucleotide
tggccagaaagaggaaga

SEQ ID NO: 245 - DNA, 18 bp, Artificial sequence
  Synthetic oligonucleotide
gccagaaagaggaagagc

SEQ ID NO: 246 - DNA, 18 bp, Artificial sequence
  Synthetic oligonucleotide
cagaaagaggaagagctg

SEQ ID NO: 247 - DNA, 18 bp, Artificial sequence
  Synthetic oligonucleotide
gaaagaggaagagctggg

SEQ ID NO: 248 - DNA, 18 bp, Artificial sequence
  Synthetic oligonucleotide
atccatccatgtactcac

SEQ ID NO: 249 - DNA, 18 bp, Artificial sequence
  Synthetic oligonucleotide
aagaggaagagctgggca

SEQ ID NO: 250 - DNA, 18 bp, Artificial sequence
  Synthetic oligonucleotide
gaggaagagctgggcagg

SEQ ID NO: 251 - DNA, 18 bp, Artificial sequence
  Synthetic oligonucleotide
ggaagagctgggcaggca

SEQ ID NO: 252 - DNA, 18 bp, Artificial sequence
  Synthetic oligonucleotide
aagagctgggcaggcagg

SEQ ID NO: 253 - DNA, 18 bp, Artificial sequence
  Synthetic oligonucleotide
gagctgggcaggcaggaa

SEQ ID NO: 254 - DNA, 18 bp, Artificial sequence
  Synthetic oligonucleotide
gctgggcaggcaggaatg

SEQ ID NO: 255 - DNA, 18 bp, Artificial sequence
  Synthetic oligonucleotide
tgggcaggcaggaatggt

SEQ ID NO: 256 - DNA, 18 bp, Artificial sequence
  Synthetic oligonucleotide
ggcaggcaggaatggtga

SEQ ID NO: 257 - DNA, 18 bp, Artificial sequence
  Synthetic oligonucleotide
caggcaggaatggtgagt

SEQ ID NO: 258 - DNA, 18 bp, Artificial sequence
  Synthetic oligonucleotide
ggcaggaatggtgagtgg

SEQ ID NO: 259 - DNA, 18 bp, Artificial sequence
  Synthetic oligonucleotide
ccatccatgtactcaccc

SEQ ID NO: 260 - DNA, 18 bp, Artificial sequence
  Synthetic oligonucleotide
caggaatggtgagtggaa

SEQ ID NO: 261 - DNA, 18 bp, Artificial sequence
  Synthetic oligonucleotide
ggaatggtgagtggaagt

SEQ ID NO: 262 - DNA, 18 bp, Artificial sequence
  Synthetic oligonucleotide
aatggtgagtggaagtgg

SEQ ID NO: 263 - DNA, 18 bp, Artificial sequence
  Synthetic oligonucleotide
tggtgagtggaagtggca

SEQ ID NO: 264 - DNA, 18 bp, Artificial sequence
  Synthetic oligonucleotide
gtgagtggaagtggcatg

SEQ ID NO: 265 - DNA, 18 bp, Artificial sequence
  Synthetic oligonucleotide
gagtggaagtggcatggg

SEQ ID NO: 266 - DNA, 18 bp, Artificial sequence
  Synthetic oligonucleotide
gtggaagtggcatgggag

SEQ ID NO: 267 - DNA, 18 bp, Artificial sequence
  Synthetic oligonucleotide
ggaagtggcatgggaggg

SEQ ID NO: 268 - DNA, 18 bp, Artificial sequence
  Synthetic oligonucleotide
aagtggcatgggaggggc

SEQ ID NO: 269 - DNA, 18 bp, Artificial sequence
  Synthetic oligonucleotide
gtggcatgggaggggccg

SEQ ID NO: 270 - DNA, 18 bp, Artificial sequence
  Synthetic oligonucleotide
atccatgtactcacccat

SEQ ID NO: 271 - DNA, 18 bp, Artificial sequence
  Synthetic oligonucleotide
ggcatgggaggggccgag

SEQ ID NO: 272 - DNA, 18 bp, Artificial sequence
  Synthetic oligonucleotide
catgggaggggccgagca

SEQ ID NO: 273 - DNA, 18 bp, Artificial sequence
  Synthetic oligonucleotide
tgggaggggccgagcaat

SEQ ID NO: 274 - DNA, 18 bp, Artificial sequence
  Synthetic oligonucleotide
ggaggggccgagcaatga

SEQ ID NO: 275 - DNA, 18 bp, Artificial sequence
  Synthetic oligonucleotide
aggggccgagcaatgacc

SEQ ID NO: 276 - DNA, 18 bp, Artificial sequence
  Synthetic oligonucleotide
gggccgagcaatgaccct

SEQ ID NO: 277 - DNA, 18 bp, Artificial sequence
  Synthetic oligonucleotide
gccgagcaatgaccctca

SEQ ID NO: 278 - DNA, 18 bp, Artificial sequence
  Synthetic oligonucleotide
cgagcaatgaccctcacc

SEQ ID NO: 279 - DNA, 18 bp, Artificial sequence
  Synthetic oligonucleotide
agcaatgaccctcacctc

SEQ ID NO: 280 - DNA, 18 bp, Artificial sequence
  Synthetic oligonucleotide
caatgaccctcacctcat

SEQ ID NO: 281 - DNA, 18 bp, Artificial sequence
  Synthetic oligonucleotide
ccatgtactcacccatct

SEQ ID NO: 282 - DNA, 18 bp, Artificial sequence
  Synthetic oligonucleotide
atgaccctcacctcatgc

SEQ ID NO: 283 - DNA, 18 bp, Artificial sequence
  Synthetic oligonucleotide
gaccctcacctcatgcag

SEQ ID NO: 284 - DNA, 18 bp, Artificial sequence
  Synthetic oligonucleotide
ccctcacctcatgcagac

SEQ ID NO: 285 - DNA, 18 bp, Artificial sequence
  Synthetic oligonucleotide
ctcacctcatgcagacag

SEQ ID NO: 286 - DNA, 18 bp, Artificial sequence
  Synthetic oligonucleotide
cacctcatgcagacaggt

SEQ ID NO: 287 - DNA, 18 bp, Artificial sequence
  Synthetic oligonucleotide
cctcatgcagacaggtgt

SEQ ID NO: 288 - DNA, 18 bp, Artificial sequence
  Synthetic oligonucleotide
tcatgcagacaggtgtac

SEQ ID NO: 289 - DNA, 18 bp, Artificial sequence
  Synthetic oligonucleotide
atgcagacaggtgtactg

SEQ ID NO: 290 - DNA, 18 bp, Artificial sequence
  Synthetic oligonucleotide
gcagacaggtgtactgga

SEQ ID NO: 291 - DNA, 18 bp, Artificial sequence
  Synthetic oligonucleotide
agacaggtgtactggaca

SEQ ID NO: 292 - DNA, 18 bp, Artificial sequence
  Synthetic oligonucleotide
atgtactcacccatctct

SEQ ID NO: 293 - DNA, 18 bp, Artificial sequence
  Synthetic oligonucleotide
acaggtgtactggacatg

SEQ ID NO: 294 - DNA, 18 bp, Artificial sequence
  Synthetic oligonucleotide
aggtgtactggacatggt

SEQ ID NO: 295 - DNA, 18 bp, Artificial sequence
  Synthetic oligonucleotide
gtgtactggacatggtac

SEQ ID NO: 296 - DNA, 18 bp, Artificial sequence
  Synthetic oligonucleotide
gtactggacatggtacgg

SEQ ID NO: 297 - DNA, 18 bp, Artificial sequence
  Synthetic oligonucleotide
actggacatggtacgggg

SEQ ID NO: 298 - DNA, 18 bp, Artificial sequence
  Synthetic oligonucleotide
tggacatggtacggggcc

SEQ ID NO: 299 - DNA, 18 bp, Artificial sequence
  Synthetic oligonucleotide
gacatggtacggggccac

SEQ ID NO: 300 - DNA, 18 bp, Artificial sequence
  Synthetic oligonucleotide
catggtacggggccacct

SEQ ID NO: 301 - DNA, 18 bp, Artificial sequence
  Synthetic oligonucleotide
tggtacggggccaccttc

SEQ ID NO: 302 - DNA, 18 bp, Artificial sequence
  Synthetic oligonucleotide
gtacggggccaccttctc

SEQ ID NO: 303 - DNA, 18 bp, Artificial sequence
  Synthetic oligonucleotide
gtactcacccatctctcc

SEQ ID NO: 304 - DNA, 18 bp, Artificial sequence
  Synthetic oligonucleotide
acggggccaccttctcct

SEQ ID NO: 305 - DNA, 18 bp, Artificial sequence
  Synthetic oligonucleotide
ggggccaccttctcctcg

SEQ ID NO: 306 - DNA, 18 bp, Artificial sequence
  Synthetic oligonucleotide
ggccaccttctcctcgcc

SEQ ID NO: 307 - DNA, 18 bp, Artificial sequence
  Synthetic oligonucleotide
ccaccttctcctcgcctt

SEQ ID NO: 308 - DNA, 18 bp, Artificial sequence
  Synthetic oligonucleotide
actcacccatctctccat

SEQ ID NO: 309 - DNA, 18 bp, Artificial sequence
  Synthetic oligonucleotide
tcacccatctctccatcc

SEQ ID NO: 310 - DNA, 18 bp, Artificial sequence
  Synthetic oligonucleotide
acccatctctccatccat

SEQ ID NO: 311 - DNA, 18 bp, Artificial sequence
  Synthetic oligonucleotide
atctagccacgaatctac

SEQ ID NO: 312 - DNA, 18 bp, Artificial sequence
  Synthetic oligonucleotide
ccatctctccatccatcc

SEQ ID NO: 313 - DNA, 18 bp, Artificial sequence
  Synthetic oligonucleotide
atctctccatccatcctt

SEQ ID NO: 314 - DNA, 18 bp, Artificial sequence
  Synthetic oligonucleotide
ctctccatccatcctttt

SEQ ID NO: 315 - DNA, 18 bp, Artificial sequence
  Synthetic oligonucleotide
ctccatccatccttttat

SEQ ID NO: 316 - DNA, 18 bp, Artificial sequence
  Synthetic oligonucleotide
ccatccatccttttatct

SEQ ID NO: 317 - DNA, 18 bp, Artificial sequence
  Synthetic oligonucleotide
atccatccttttatctac

SEQ ID NO: 318 - DNA, 18 bp, Artificial sequence
  Synthetic oligonucleotide
ccatccttttatctactc

SEQ ID NO: 319 - DNA, 18 bp, Artificial sequence
  Synthetic oligonucleotide
atccttttatctactcat

SEQ ID NO: 320 - DNA, 18 bp, Artificial sequence
  Synthetic oligonucleotide
ccttttatctactcatca

SEQ ID NO: 321 - DNA, 18 bp, Artificial sequence
  Synthetic oligonucleotide
ttttatctactcatcact

SEQ ID NO: 322 - DNA, 18 bp, Artificial sequence
  Synthetic oligonucleotide
ctagccacgaatctaccc

SEQ ID NO: 323 - DNA, 18 bp, Artificial sequence
  Synthetic oligonucleotide
ttatctactcatcactca

SEQ ID NO: 324 - DNA, 18 bp, Artificial sequence
  Synthetic oligonucleotide
atctactcatcactcatt

SEQ ID NO: 325 - DNA, 18 bp, Artificial sequence
  Synthetic oligonucleotide
ctactcatcactcattca

SEQ ID NO: 326 - DNA, 18 bp, Artificial sequence
  Synthetic oligonucleotide
actcatcactcattcatc

SEQ ID NO: 327 - DNA, 18 bp, Artificial sequence
  Synthetic oligonucleotide
tcatcactcattcatctg

SEQ ID NO: 328 - DNA, 18 bp, Artificial sequence
  Synthetic oligonucleotide
atcactcattcatctgtt

SEQ ID NO: 329 - DNA, 18 bp, Artificial sequence
  Synthetic oligonucleotide
cactcattcatctgttca

SEQ ID NO: 330 - DNA, 18 bp, Artificial sequence
  Synthetic oligonucleotide
ctcattcatctgttcaat

SEQ ID NO: 331 - DNA, 18 bp, Artificial sequence
  Synthetic oligonucleotide
cattcatctgttcaatca

SEQ ID NO: 332 - DNA, 18 bp, Artificial sequence
  Synthetic oligonucleotide
ttcatctgttcaatcatt

(19) United States
(12) Patent Application Publication
(10) Pub. No.: US 2024/0301415 A1
(43) Pub. Date: Sep. 12, 2024

(54) COMPOUNDS FOR MODULATING UNC13A EXPRESSION

(71) Applicant: Ionis Pharmaceuticals, Inc., Carlsbad, CA (US)

(72) Inventors: Ruben E. Valas, Oceanside, CA (US); Kar Yun Karen Ling, Carlsbad, CA (US)

(73) Assignee: Ionis Pharmaceuticals, Inc., Carlsbad, CA (US)

(21) Appl. No.: 18/562,543

(22) PCT Filed: May 20, 2022
(86) PCT No.: PCT/US2022/030335

§ 371 Date: Nov. 20, 2023

Related U.S. Application Data
(60) Provisional application No. 63/191,905, filed on May 21, 2021.

(51) Int. Cl. C12N 15/113

(52) U.S. Cl. CPC C12N 15/113; C12N 2310/11; C12N 2310/315; C12N 2310/321; C12N 2310/322; C12N 2310/3231; C12N 2310/3341; C12N 2310/351

(57) ABSTRACT

Provided are oligomeric compounds and pharmaceutical compositions for increasing the amount or activity of UNC13A RNA in a cell or animal, and/or decreasing the amount of UNC13A RNA that includes a cryptic exon in a cell or animal, and in certain instances increasing the amount of UNC13A protein in a cell or animal. Such oligomeric compounds and pharmaceutical compositions are useful to treat neurodegenerative diseases such as amyotrophic lateral sclerosis and frontotemporal dementia.

### SEQUENCE LISTING

The present application is being filed along with a Sequence Listing in electronic format. The Sequence Listing is provided as a file entitled BIOL0426WOSEQ_ST25.txt, created on May 20, 2022, which is 196 KB in size. The information in the electronic format of the sequence listing is incorporated herein by reference in its entirety.

### FIELD

Provided are oligomeric agents, oligomeric compounds, antisense agents, and pharmaceutical compositions for increasing the amount or activity of UNC13A RNA in a cell or animal, and/or decreasing the amount of UNC13A RNA that includes a cryptic exon in a cell or animal, and in certain instances increasing the amount of UNC13A protein in a cell or animal. Such oligomeric agents, oligomeric compounds, antisense agents, and pharmaceutical compositions are useful to treat neurodegenerative diseases such as amyotrophic lateral sclerosis (ALS) and frontotemporal dementia (FTD).

### BACKGROUND

The UNC13A gene encodes UNC13A, a member of the UNC13 family of proteins, which are involved in calcium-triggered synaptic vesicle release (Dittman, J. S., 2019, Curr. Opin. Neurobiol. 57, 17-25). Mutations in UNC13A have been associated with neurodegenerative diseases such as amyotrophic lateral sclerosis (ALS), and frontotemporal dementia (FTD) (Diekstra, F. P., et al., 2014, Ann. Neurol. 76:120-133).

ALS, also known as Lou Gehrig's disease, is a disorder characterized by a selective degeneration of upper and lower motor neurons (Rowland, N. Engl. J. Med. 2001, 344, 1688-1700). ALS is a devastating progressive neurodegenerative disease affecting as many as 30,000 Americans at any given time. The progressive degeneration of the motor neurons in ALS eventually leads to the patient's death. When the motor neurons die, the ability of the brain to initiate and control muscle movement is lost. With voluntary muscle action progressively affected, patients in the later stages of the disease may become totally paralyzed.

FTD refers to a group of disorders caused by progressive nerve cell loss in the brain's frontal lobes or temporal lobes. Nerve cell damage caused by FTD leads to loss of function in the frontal lobes or temporal lobes, which variably cause deterioration in behavior and personality, language disturbances, or alterations in muscle or motor functions.

Both ALS and FTD are neurodegenerative diseases associated with TDP-43 proteinopathies. TDP-43 represses the inclusion of cryptic exons that cause nonsense-mediated decay in RNA; the loss of TDP-43 increases the amount of cryptic exon-including RNA, causing nonsense-mediated decay of the transcript. In both ALS and FTD, the RNA binding protein TDP-43 is depleted from the nucleus of neurons in the brain and spinal cord, reducing the expression of genes that contain cryptic exons, and leading to cell death. (Ling, P. et al., 2015, Science 349, 650-655; Humphrey, et al., 2017, BMC Medical Genomics 10, 38).

The UNC13A gene contains a cryptic exon that promotes nonsense-mediated decay. When TDP-43 levels are depleted, the cryptic exon is included in the transcript, resulting in insufficient UNC13A protein expression. ALS- and FTD-associated single nucleotide polymorphisms (SNPs) in UNC13A are associated with an increased risk of cryptic exon inclusion in the UNC13A transcript (Brown, A-L., et al., 2021, bioRxiv, doi.org/10.1101/2021.04.02.438170; Ma., X. R., et al., 2021, bioRxiv, doi.org/10.1101/2021.04.02.438213, also published in Ma., X. R., et al., 2022, Nature 603, 124-130).

Currently there is a lack of acceptable options for treating ALS, FTD, and other neurodegenerative diseases. It is therefore an object herein to provide oligomeric agents, oligomeric compounds, antisense agents, and pharmaceutical compositions for the treatment of such diseases.

### SUMMARY

Oligomeric agents, oligomeric compounds, antisense agents, and pharmaceutical compositions of certain embodiments described herein are useful for increasing UNC13A expression in a cell or animal. In certain embodiments, the oligomeric agents, oligomeric compounds, antisense agents, and pharmaceutical compositions increase UNC13A RNA or protein levels in a cell or animal. In certain embodiments, the oligomeric agents, oligomeric compounds, antisense agents, and pharmaceutical compositions decrease the amount of UNC13A RNA that includes a cryptic exon in a cell or animal. In certain embodiments, the animal is a subject having a neurodegenerative disease, in certain embodiments, the subject has ALS or FTD. In some embodiments, the subject has ALS. In some embodiments, the subject has FTD.

Also provided are methods useful for ameliorating at least one symptom of a neurodegenerative disease. In certain embodiments, the neurodegenerative disease is amyotrophic lateral sclerosis (ALS) or frontotemporal dementia (FTD). In certain embodiments, symptoms include motor dysfunction, muscle weakness, muscle wasting, synaptic dysfunction, fatigue, difficulty speaking, difficulty swallowing, shortness of breath, cognitive impairment, or decreased longevity. In certain embodiments, amelioration of these symptoms results in improved motor function, improved muscle strength, increased muscle mass, improved speaking, improved swallowing, improved breathing, improved synaptic function, improved cognition, or increased longevity.

### DETAILED DESCRIPTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

As used herein, “2′-deoxynucleoside” means a nucleoside comprising a 2′-H(H) deoxyfuranosyl sugar moiety. In certain embodiments, a 2′-deoxynucleoside is a 2′-β-D-deoxynucleoside and comprises a 2′-β-D-deoxyribosyl sugar moiety, which has the β-D ribosyl configuration as found in naturally occurring deoxyribonucleic acids (DNA). In certain embodiments, a 2′-deoxynucleoside may comprise a modified nucleobase or may comprise an RNA nucleobase (uracil).

As used herein, “2′-MOE” means a 2′-OCH₂CH₂OCH₃ group in place of the 2′—OH group of a furanosyl sugar moiety. A “2′-MOE sugar moiety” means a sugar moiety with a 2′-OCH₂CH₂OCH₃ group in place of the 2′—OH group of a furanosyl sugar moiety. Unless otherwise indicated, a 2′-MOE sugar moiety is in the β-D-ribosyl configuration. “MOE” means O-methoxyethyl.

As used herein, “2′-MOE nucleoside” means a nucleoside comprising a 2′-MOE sugar moiety.

As used herein, “2′-NMA” means a —O—CH₂—C(═O)—NH—CH₃ group in place of the 2′—OH group of a ribosyl sugar moiety. A “2′-NMA sugar moiety” is a sugar moiety with a 2′-O—CH₂—C(═O)—NH—CH₃ group in place of the 2′-OH group of a ribosyl sugar moiety. Unless otherwise indicated, a 2′-NMA sugar moiety is in the β-D configuration. “NMA” means O—N-methyl acetamide.

As used herein, “2′-NMA nucleoside” means a nucleoside comprising a 2′-NMA sugar moiety.

As used herein, “2′-OMe” means a 2′-OCH₃ group in place of the 2′—OH group of a furanosyl sugar moiety. A “2′-O-methyl sugar moiety” or “2′-OMe sugar moiety” means a sugar moiety with a 2′-OCH₃ group in place of the 2′-OH group of a furanosyl sugar moiety. Unless otherwise indicated, a 2′-OMe sugar moiety is in the β-D-ribosyl configuration.

As used herein, “2′-OMe nucleoside” means a nucleoside comprising a 2′-OMe sugar moiety.

As used herein, “2′-F” means a 2′-fluoro group in place of the 2′—OH group of a ribosyl sugar moiety. A “2′-F sugar moiety” or “2′-fluororibosyl sugar moiety” means a sugar moiety with a 2′—F group in place of the 2′—OH group of a ribosyl sugar moiety. Unless otherwise indicated, a 2′-F has the β-D ribosyl stereochemical configuration.

As used herein, “2′-F nucleoside” means a nucleoside comprising a 2′-F sugar moiety.

As used herein, “2′-substituted nucleoside” means a nucleoside comprising a 2′-substituted sugar moiety. As used herein, “2′-substituted” in reference to a sugar moiety means a sugar moiety comprising at least one 2′-substituent group other than H or OH.

As used herein, “3′ target site” refers to the 3′-most nucleotide of a target nucleic acid which is complementary to an antisense oligonucleotide, when the antisense oligonucleotide is hybridized to the target nucleic acid.

As used herein, “5′ target site” refers to the 5′-most nucleotide of a target nucleic acid which is complementary to an antisense oligonucleotide, when the antisense oligonucleotide is hybridized to the target nucleic acid.

As used herein, “5-methylcytosine” means a cytosine modified with a methyl group attached to the 5 position. A 5-methylcytosine is a modified nucleobase.

As used herein, “abasic sugar moiety” means a sugar moiety of a nucleoside that is not attached to a nucleobase. Such abasic sugar moieties are sometimes referred to in the art as “abasic nucleosides.”

As used herein, “administration” or “administering” means providing a pharmaceutical agent or composition to an animal.

As used herein, “ameliorate” in reference to a treatment means improvement in at least one symptom or hallmark relative to the same symptom or hallmark in the absence of the treatment. In certain embodiments, amelioration is the reduction in the severity or frequency of a symptom or hallmark or the delayed onset or slowing of progression in the severity or frequency of a symptom or hallmark. The progression or severity of indicators may be determined by subjective or objective measures, which are known to those skilled in the art.

As used herein, “animal” means a human or non-human animal.

As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the furanosyl sugar moiety is a ribosyl sugar moiety. In certain embodiments, the bicyclic sugar moiety does not comprise a furanosyl moiety.

As used herein, “bicyclic nucleoside” or “BNA” means a nucleoside comprising a bicyclic sugar moiety.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are modified oligonucleotides. In certain embodiments, the molecules are oligomeric compounds comprising modified oligonucleotides.

As used herein, “cerebrospinal fluid” or “CSF” means the fluid filling the space around the brain and spinal cord. “Artificial cerebrospinal fluid” or “aCSF” means a prepared or manufactured fluid that has certain properties (e.g., osmolarity, pH, and/or electrolytes) similar to cerebrospinal fluid and is biocompatible with CSF.

As used herein, “cleavable moiety” means a bond or group of atoms that is cleaved under physiological conditions, for example, inside a cell, an animal, or a human.

As used herein, “complementary” in reference to an oligonucleotide means that at least 70% of the nucleobases of the oligonucleotide and the nucleobases of another nucleic acid or one or more regions thereof are capable of hydrogen bonding with one another when the nucleobase sequence of the oligonucleotide and the other nucleic acid are aligned in opposing directions. “Complementary region” in reference to a region of an oligonucleotide means that at least 70% of the nucleobases of that region and the nucleobases of another nucleic acid or one or more regions thereof are capable of hydrogen bonding with one another when the nucleobase sequence of the oligonucleotide and the other nucleic acid are aligned in opposing directions. “Complementary nucleobases” means nucleobases that are capable of forming hydrogen bonds with one another. Complementary nucleobase pairs include adenine (A) and thymine (T), adenine (A) and uracil (U), cytosine (C) and guanine (G), 5-methylcytosine (mC) and guanine (G). Certain modified nucleobases that pair with natural nucleobases or with other modified nucleobases are known in the art and are not considered complementary nucleobases as defined herein unless indicated otherwise. For example, inosine can pair, but is not considered complementary, with adenosine, cytosine, or uracil. Complementary oligonucleotides and/or nucleic acids need not have nucleobase complementarity at each nucleoside. Rather, some mismatches are tolerated. As used herein, “fully complementary” or “100% complementary” in reference to oligonucleotides means that oligonucleotides are complementary to another oligonucleotide or nucleic acid at each nucleoside of the oligonucleotide.

As used herein, “cryptic exon” or “nonsense-mediated decay (NMD) exon” is an exon, or a pseudo-exon, that, when included in an mRNA transcript can activate the nonsense-mediated decay (NMD) pathway.

As used herein, “conjugate group” means a group of atoms that is directly attached to an oligonucleotide. Conjugate groups include a conjugate moiety and a conjugate linker that attaches the conjugate moiety to the oligonucleotide.

As used herein, “conjugate linker” means a single bond or a group of atoms comprising at least one bond that connects a conjugate moiety to an oligonucleotide.

As used herein, “conjugate moiety” means a group of atoms that modifies one or more properties of a molecule compared to the identical molecule lacking the conjugate moiety, including but not limited to pharmacodynamics, pharmacokinetics, stability, binding, absorption, tissue distribution, cellular distribution, cellular uptake, charge and clearance.

As used herein, “constrained ethyl” or “cEt” or “cEt modified sugar moiety” or “cEt sugar moiety” means a β-D ribosyl bicyclic sugar moiety wherein the second ring of the bicyclic sugar is formed via a bridge connecting the 4′-carbon and the 2′-carbon of the β-D ribosyl sugar moiety, wherein the bridge has the formula 4′-CH(CH)—O-2′, and wherein the methyl group of the bridge is in the S configuration.

As used herein, “cEt nucleoside” means a nucleoside comprising a cEt modified sugar moiety.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “diluent” means an ingredient in a composition that lacks pharmacological activity, but is pharmaceutically necessary or desirable. For example, the diluent in an injected composition can be a liquid, e.g., aCSF, PBS, or saline solution.

As used herein, “double-stranded” in reference to a region or an oligonucleotide means a duplex formed by complementary strands of nucleic acids (including, but not limited to oligonucleotides) hybridized to one another. In certain embodiments, the two strands of a double-stranded region are separate molecules. In certain embodiments, the two strands are regions of the same molecule that has folded onto itself (e.g., a hairpin structure).

As used herein, “hotspot region” is a range of nucleobases on a target nucleic acid that is amenable to oligomeric agent or oligomeric compound-mediated reduction of the amount or activity of the target nucleic acid.

As used herein, “internucleoside linkage” is the covalent linkage between adjacent nucleosides in an oligonucleotide. As used herein “modified internucleoside linkage” means any internucleoside linkage other than a phosphodiester internucleoside linkage. “Phosphorothioate internucleoside linkage” or “PS internucleoside linkage” is a modified internucleoside linkage in which one of the non-bridging oxygen atoms of a phosphodiester internucleoside linkage is replaced with a sulfur atom.

As used herein, “inverted nucleoside” means a nucleotide having a 3′ to 3′ and/or 5′ to 5′ internucleoside linkage, as shown herein.

As used herein, “inverted sugar moiety” means the sugar moiety of an inverted nucleoside or an abasic sugar moiety having a 3′ to 3′ and/or 5′ to 5′ internucleoside linkage.

As used herein, “linked nucleosides” are nucleosides that are connected in a contiguous sequence (i.e., no additional nucleosides are presented between those that are linked).

As used herein, “linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

As used herein, “mismatch” or “non-complementary” means a nucleobase of a first nucleic acid sequence that is not complementary with the corresponding nucleobase of a second nucleic acid sequence or target nucleic acid when the first and second nucleic acid sequences are aligned in opposing directions.

As used herein, “motif” means the pattern of unmodified and/or modified sugar moieties, nucleobases, and/or internucleoside linkages, in an oligonucleotide.

As used herein, “modified nucleoside” means a nucleoside comprising a modified nucleobase and/or a modified sugar moiety. Modified nucleosides include abasic nucleosides, which lack a nucleobase.

As used herein, “non-bicyclic modified sugar moiety” means a modified sugar moiety that comprises a modification, such as a substituent, that does not form a bridge between two atoms of the sugar to form a second ring.

As used herein, “nucleobase” means an unmodified nucleobase or a modified nucleobase. A nucleobase is a heterocyclic moiety. As used herein an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one other nucleobase. A “5-methylcytosine” is a modified nucleobase. A universal base is a modified nucleobase that can pair with any one of the five unmodified nucleobases.

As used herein, “nucleobase sequence” means the order of contiguous nucleobases in a nucleic acid or oligonucleotide independent of any sugar or internucleoside linkage modification.

As used herein, “nucleoside” means a compound or fragment of a compound comprising a nucleobase and a sugar moiety. The nucleobase and sugar moiety are each, independently, unmodified or modified.

As used herein, “oligomeric agent” means an oligomeric compound and optionally one or more additional features, such as a second oligomeric compound. An oligomeric agent may be a single-stranded oligomeric compound or may be an oligomeric duplex formed by two complementary oligomeric compounds.

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound.

The term “oligomeric duplex” means a duplex formed by two oligomeric compounds having complementary nucleobase sequences.

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to an animal. Certain such carriers enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water, sterile saline, sterile buffer solution or sterile artificial cerebrospinal fluid.

As used herein “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein “pharmaceutical composition” means a mixture of substances suitable for administering to a subject. For example, a pharmaceutical composition may comprise an oligomeric compound and a sterile aqueous solution. In certain embodiments, a pharmaceutical composition shows activity in free uptake assay in certain cell lines.

As used herein “prodrug” means a therapeutic agent in a first form outside the body that is converted to a second form within an animal or cells thereof. Typically, conversion of a prodrug within the animal is facilitated by the action of an enzymes (e.g., endogenous or viral enzyme) or chemicals present in cells or tissues and/or by physiologic conditions. In certain embodiments, the first form of the prodrug is less active than the second form.

As used herein, “single-stranded” means a nucleic acid (including but not limited to an oligonucleotide) that is unpaired and is not part of a duplex. Single-stranded compounds are capable of hybridizing with complementary nucleic acids to form duplexes, at which point they are no longer single-stranded.

As used herein, “stabilized phosphate group” refers to a 5′-chemical moiety that results in stabilization of a 5′-phosphate moiety of the 5′-terminal nucleoside of an oligonucleotide, relative to the stability of an unmodified 5′-phosphate of an unmodified nucleoside under biologic conditions. Such stabilization of a 5′-phosphate group includes but is not limited to resistance to removal by phosphatases. Stabilized phosphate groups include, but are not limited to, 5′-vinyl phosphonates and 5′-cyclopropyl phosphonate.

As used herein, “standard cell assay” means the assays described in Example 2 or Example 3 and reasonable variations thereof.

As used herein, “stereorandom chiral center” in the context of a population of molecules of identical molecular formula means a chiral center having a random stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center. The stereochemical configuration of a chiral center is considered random when it is the result of a synthetic method that is not designed to control the stereochemical configuration. In certain embodiments, a stereorandom chiral center is a stereorandom phosphorothioate internucleoside linkage.

As used herein, “subject” means a human or non-human animal. In certain embodiments, the subject is a human.

As used herein, “sugar moiety” means an unmodified sugar moiety or a modified sugar moiety. As used herein, “unmodified sugar moiety” means a 2′-OH(H) ribosyl moiety, as found in RNA (an “unmodified RNA sugar moiety”), or a 2′-H(H) deoxyribosyl sugar moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate.

As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or target nucleic acids.

As used herein, “symptom or hallmark” means any physical feature or test result that indicates the existence or extent of a disease or disorder. In certain embodiments, a symptom is apparent to a subject or to a medical professional examining or testing said subject. In certain embodiments, a hallmark is apparent upon invasive diagnostic testing, including, but not limited to, post-mortem tests.

As used herein, “target nucleic acid” and “target RNA” mean a nucleic acid that an oligomeric compound is designed to affect. Target RNA means an RNA transcript and includes pre-mRNA and mature mRNA unless otherwise specified.

As used herein, “target region” means a portion of a target nucleic acid to which an oligomeric compound is designed to hybridize.

As used herein, “terminal group” means a chemical group or group of atoms that is covalently linked to a terminus of an oligonucleotide.

As used herein, “treating” means improving a subject's disease or condition by administering an oligomeric agent, an oligomeric compound, an oligomeric duplex, or an antisense agent described herein. In certain embodiments, treating a subject improves a symptom relative to the same symptom in the absence of the treatment. In certain embodiments, treatment reduces in the severity or frequency of a symptom, or delays the onset of a symptom, slows the progression of a symptom, or slows the severity or frequency of a symptom.

As used herein, “RNA” means an RNA transcript and includes pre-mRNA and mature mRNA unless otherwise specified.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound. In certain embodiments, antisense activity is the modulation of splicing of a target pre-mRNA.

As used herein, “antisense agent” means an antisense compound and optionally one or more additional features, such as a sense compound.

As used herein, “antisense compound” means an antisense oligonucleotide and optionally one or more additional features, such as a conjugate group.

As used herein, “sense compound” means a sense oligonucleotide and optionally one or more additional features, such as a conjugate group.

As used herein, “antisense oligonucleotide” means an oligonucleotide, including the oligonucleotide portion of an antisense compound, that is capable of hybridizing to a target nucleic acid and is capable of at least one antisense activity. Antisense oligonucleotides include but are not limited to splice-modulating oligonucleotides, antisense RNAi oligonucleotides and antisense RNase H oligonucleotides.

As used herein, “sense oligonucleotide” means an oligonucleotide, including the oligonucleotide portion of a sense compound, that is capable of hybridizing to an antisense oligonucleotide.

As used herein, “cell-targeting moiety” means a conjugate group or portion of a conjugate group that is capable of binding to a particular cell type or particular cell types.

As used herein, “hybridization” means the annealing of oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases. In certain embodiments, complementary nucleic acid molecules include, but are not limited to, an antisense compound and a nucleic acid target. In certain embodiments, complementary nucleic acid molecules include, but are not limited to, an oligonucleotide and a nucleic acid target.

As used herein, “RNAi agent” means an antisense agent that acts, at least in part, through RISC or Ago2 to modulate a target nucleic acid and/or protein encoded by a target nucleic acid. RNAi agents include, but are not limited to double-stranded siRNA, single-stranded RNAi (ssRNAi), and microRNA, including microRNA mimics. RNAi agents may comprise conjugate groups and/or terminal groups. In certain embodiments, an RNAi agent modulates the amount and/or activity, of a target nucleic acid. The term RNAi agent excludes antisense agents that act through RNase H.

As used herein, “RNase H agent” means an antisense agent that acts through RNase H to modulate a target nucleic acid and/or protein encoded by a target nucleic acid. In certain embodiments, RNase H agents are single-stranded. In certain embodiments, RNase H agents are double-stranded. RNase H compounds may comprise conjugate groups and/or terminal groups. In certain embodiments, an RNase H agent modulates the amount and/or activity of a target nucleic acid. The term RNase H agent excludes antisense agents that act principally through RISC/Ago2.

### CERTAIN EMBODIMENTS

Embodiment 1. An oligomeric compound comprising a modified oligonucleotide consisting of 8 to 80 linked nucleosides, wherein the nucleobase sequence of the modified oligonucleotide is at least 80% complementary to an equal length portion of a UNC13A nucleic acid, and wherein the modified oligonucleotide has at least one modification selected from a modified sugar moiety and a modified internucleoside linkage.

Embodiment 2. The oligomeric compound of embodiment 1, wherein the UNC13A nucleic acid has the nucleobase sequence of SEQ ID NO: 1 or SEQ ID NO: 2.

Embodiment 3. The oligomeric compound of embodiment 1 or embodiment 2, wherein the nucleobase sequence of the modified oligonucleotide is at least 80% complementary to an equal length portion within nucleobases 48,128-48,151, nucleobases 48,432-48,465, or nucleobases 48,466-48,561 of SEQ ID NO: 1.

Embodiment 4. The oligomeric compound of any of embodiments 1-3, wherein the nucleobase sequence of the modified oligonucleotide is at least 85%, at least 90%, at least 95%, or 100% complementary to the nucleobase sequence of an equal length portion of the UNC13A nucleic acid.

Embodiment 5. An oligomeric compound comprising a modified oligonucleotide consisting of 8 to 80 linked nucleosides, wherein the nucleobase sequence of the modified oligonucleotide comprises at least 8, at least 9, at least 10, at least 11, at least 12, at least 13, at least 14, at least 15, at least 16, at least 17, or 18 contiguous nucleobases of any of the nucleobase sequences of SEQ ID NOs: 21-332, and wherein the modified oligonucleotide comprises at least one modification selected from a modified sugar moiety and a modified internucleoside linkage.

Embodiment 6. The oligomeric compound of embodiment 5, wherein the nucleobase sequence of the modified oligonucleotide comprises the nucleobase sequence of any of SEQ ID NOs: 21-332.

Embodiment 7. The oligomeric compound of embodiment 5, wherein the modified oligonucleotide has a nucleobase sequence consisting of the nucleobase sequence of any of SEQ ID NOs: 21-332.

Embodiment 8. The oligomeric compound of any one of embodiments 5-7, wherein the modified oligonucleotide has a nucleobase sequence comprising at least 8, at least 9, at least 10, at least 11, at least 12, at least 13, at least 14, at least 15, at least 16, at least 17, or 18 contiguous nucleobases of any of the nucleobase sequences of:
SEQ ID NOs: 290, 291, 293, or 294;
SEQ ID NOs: 85-101; or
SEQ ID NOs: 21-30, 32-41, 43-52, 54-60, 62-66, or 326-332.

Embodiment 9. The oligomeric compound of any of embodiments 5-8, wherein the nucleobase sequence of the modified oligonucleotide is at least 85%, at least 90%, at least 95%, or 100% complementary to the nucleobase sequence of an equal length portion of a UNC13A nucleic acid, wherein the UNC13A nucleic acid has the nucleobase sequence of SEQ ID NO: 1 or SEQ ID NO: 2.

Embodiment 10. An oligomeric compound comprising a modified oligonucleotide consisting of 12 to 50 linked nucleosides, wherein the nucleobase sequence of the modified oligonucleotide comprises at least 8, at least 9, at least 10, at least 11, at least 12, at least 13, at least 14, at least 15, at least 16, at least 17, or 18 contiguous nucleobases complementary to:
48,128-48,151 of SEQ ID NO: 1;
48,432-48,465 of SEQ ID NO: 1; or
48,466-48,561 of SEQ ID NO: 1.

Embodiment 11. The oligomeric compound of any of embodiments 1-10, wherein the modified oligonucleotide consists of 10 to 25, 10 to 30, 10 to 50, 12 to 20, 12 to 25, 12 to 30, 12 to 50, 13 to 20, 13 to 25, 13 to 30, 13 to 50, 14 to 20, 14 to 25, 14 to 30, 14 to 50, 15 to 20, 15 to 25, 15 to 30, 15 to 50, 16 to 18, 16 to 20, 16 to 25, 16 to 30, 16 to 50, 17 to 20, 17 to 25, 17 to 30, 17 to 50, 18 to 20, 18 to 22, 18 to 25, 18 to 30, 18 to 50, 19 to 20, 19 to 25, 19 to 30, 19 to 50, 20 to 25, 20 to 30, 20 to 50, 21 to 25, 21 to 30, 21 to 50, 22 to 25, 22 to 30, 22 to 50, 23 to 25, 23 to 30, or 23 to 50 linked nucleosides.

Embodiment 12. The oligomeric compound of any of embodiments 1-11, wherein the modified oligonucleotide consists of 18 linked nucleosides.

Embodiment 13. The oligomeric compound of any of embodiments 1-12, wherein at least one nucleoside of the modified oligonucleotide comprises a modified sugar moiety.

Embodiment 14. The oligomeric compound of embodiment 13, wherein the modified sugar moiety comprises a bicyclic sugar moiety.

Embodiment 15. The oligomeric compound of embodiment 14, wherein the bicyclic sugar moiety comprises a 2′-4′ bridge selected from —O—CH₂—; and —O—CH(CH₃)—.

Embodiment 16. The oligomeric compound of embodiment 13, wherein the modified sugar moiety comprises a non-bicyclic modified sugar moiety.

Embodiment 17. The oligomeric compound of embodiment 16, wherein the non-bicyclic modified sugar moiety is a 2′-MOE sugar moiety, a 2′-OMe sugar moiety, a 2′-NMA sugar moiety, or a 2′-F sugar moiety.

Embodiment 18. The oligomeric compound of any of embodiments 1-17, wherein at least one nucleoside of the modified oligonucleotide compound comprises a sugar surrogate.

Embodiment 19. The oligomeric compound of any of embodiments 13-18, wherein each nucleoside of the modified oligonucleotide comprises a modified sugar moiety.

Embodiment 20. The oligomeric compound of embodiment 19, wherein each modified sugar moiety is a 2′-MOE sugar moiety.

Embodiment 21. The oligomeric compound of embodiment 19, wherein each modified sugar moiety is a 2′-NMA sugar moiety.

Embodiment 22. The oligomeric compound of any of embodiments 1-21, wherein the modified oligonucleotide comprises at least one modified internucleoside linkage.

Embodiment 23. The oligomeric compound of embodiment 22, wherein at least one modified internucleoside linkage is a phosphorothioate internucleoside linkage.

Embodiment 24. The oligomeric compound of embodiment 22 or embodiment 23, wherein each internucleoside linkage is a modified internucleoside linkage.

Embodiment 25. The oligomeric compound of embodiment 24, wherein each internucleoside linkage is a phosphorothioate internucleoside linkage.

Embodiment 26. The oligomeric compound of any of embodiments 22-23, wherein at least one internucleoside linkage of the modified oligonucleotide is a phosphodiester internucleoside linkage.

Embodiment 27. The oligomeric compound of any of embodiments 1-24 or 26, wherein each internucleoside linkage of the modified oligonucleotide is independently selected from a phosphodiester or a phosphorothioate internucleoside linkage.

Embodiment 28. The oligomeric compound of any of embodiments 1-23, or 26-27, wherein at least 10, at least 11, at least 12, at least 13, at least 14, at least 15, at least 16, or 17 internucleoside linkages of the modified oligonucleotide are phosphorothioate internucleoside linkages.

Embodiment 29. The oligomeric compound of any of embodiments 1-28, wherein the modified oligonucleotide comprises at least one modified nucleobase.

Embodiment 30. The oligomeric compound of embodiment 29, wherein the modified nucleobase is 5-methylcytosine.

Embodiment 31. The oligomeric compound of embodiment 30, wherein each cytosine is a 5-methylcytosine.

Embodiment 32. The oligomeric compound of any of embodiments 1-31, consisting of the modified oligonucleotide.

Embodiment 33. The oligomeric compound of any one of embodiments 1-32, wherein the modified oligonucleotide is a pharmaceutically acceptable salt thereof.

Embodiment 34. The oligomeric compound of embodiment 33, which is a pharmaceutically acceptable salt comprising one or more cations selected from sodium, potassium, calcium, and magnesium.

Embodiment 35. The oligomeric compound of any of embodiments 1-31, wherein the oligomeric compound comprises a conjugate group.

Embodiment 36. The oligomeric compound of embodiment 35, wherein the conjugate group comprises a conjugate linker and a conjugate moiety.

Embodiment 37. The oligomeric compound of embodiment 36, wherein the conjugate linker consists of a single bond.

Embodiment 38. The oligomeric compound of embodiment 36 or embodiment 37, wherein the conjugate linker is cleavable.

Embodiment 39. The oligomeric compound of any of embodiments 36-38, wherein the conjugate linker comprises 1-3 linker-nucleosides.

Embodiment 40. The oligomeric compound of any of embodiments 36-38, wherein the conjugate linker does not comprise any linker nucleosides.

Embodiment 41. The oligomeric compound of any of embodiments 35-40, wherein the conjugate group is attached to the modified oligonucleotide at the 5′-end of the modified oligonucleotide.

Embodiment 42. The oligomeric compound of any of embodiments 35-40, wherein the conjugate group is attached to the modified oligonucleotide at the 3′-end of the modified oligonucleotide.

Embodiment 43. The oligomeric compound of any of embodiments 1 to 42, wherein the oligomeric compound comprises a terminal group.

Embodiment 44. The oligomeric compound of embodiment 43 wherein the terminal group is an abasic sugar moiety.

Embodiment 45. The oligomeric compound of any one of embodiments 1-44 wherein the oligomeric compound is a singled-stranded oligomeric compound.

Embodiment 46. A chirally enriched population of oligomeric compounds of any of embodiments 1-45, wherein the population is enriched for modified oligonucleotides comprising at least one particular phosphorothioate internucleoside linkage having a particular stereochemical configuration.

Embodiment 47. The chirally enriched population of embodiment 46, wherein the population is enriched for modified oligonucleotides comprising at least one particular phosphorothioate internucleoside linkage having the (Sp) or (Rp) configuration.

Embodiment 48. The chirally enriched population of embodiment 46, wherein the population is enriched for modified oligonucleotides having a particular, independently selected stereochemical configuration at each phosphorothioate internucleoside linkage.

Embodiment 49. The chirally enriched population of embodiment 46, wherein the population is enriched for modified oligonucleotides having the (Rp) configuration at one particular phosphorothioate internucleoside linkage and the (Sp) configuration at each of the remaining phosphorothioate internucleoside linkages.

Embodiment 50. The chirally enriched population of embodiment 46, wherein the population is enriched for modified oligonucleotides having at least 3 contiguous phosphorothioate internucleoside linkages in the Sp, Sp, and Rp configurations, in the 5′ to 3′ direction.

Embodiment 51. A population of oligomeric compounds of any of embodiments 1-45, wherein all of the phosphorothioate internucleoside linkages of the modified oligonucleotide are stereorandom.

Embodiment 52. An oligomeric duplex, comprising a first oligomeric compound and a second oligomeric compound comprising a second modified oligonucleotide, wherein the first oligomeric compound is an oligomeric compound of any of embodiments 1-45.

Embodiment 53. The oligomeric duplex of embodiment 52, wherein the second modified oligonucleotide consists of 8 to 80 linked nucleosides, and wherein the second modified oligonucleotide comprises a complementary region of at least 8 nucleobases that is at least 90% complementary to an equal length portion of the first modified oligonucleotide.

Embodiment 54. An antisense agent comprising an antisense compound, wherein the antisense compound is the oligomeric compound of any of embodiments 1-45 or an oligomeric duplex of embodiment 52 or embodiment 53.

Embodiment 55. The antisense agent of embodiment 54, wherein the antisense agent is a splice-modulating agent capable of modulating splicing of UNC13A nucleic acid.

Embodiment 56. The antisense agent of embodiment 54 or embodiment 55, wherein the antisense agent comprises a conjugate group, wherein the conjugate group comprises a cell-targeting moiety.

Embodiment 57. A pharmaceutical composition comprising an oligomeric compound of any of embodiments 1-45, a population of any of embodiments 46-51, an oligomeric duplex of embodiment 52 or embodiment 53, or an antisense agent of any of embodiments 54-56, and a pharmaceutically acceptable diluent or carrier.

Embodiment 58. The pharmaceutical composition of embodiment 57, wherein the pharmaceutically acceptable diluent is phosphate-buffered saline or artificial cerebrospinal fluid.

Embodiment 59. The pharmaceutical composition of embodiment 58, wherein the pharmaceutical composition consists essentially of the oligomeric compound, the population, the oligomeric duplex, or the antisense agent, and phosphate-buffered saline or artificial cerebrospinal fluid.

Embodiment 60. A method comprising administering to a subject an oligomeric compound of any of embodiments 1-45, a population of any of embodiments 46-51, an oligomeric duplex of embodiment 52 or embodiment 53, an antisense agent of any of embodiments 54-56, or a pharmaceutical composition of any of embodiments 57-59.

Embodiment 61. A method of treating a disease associated with UNC13A comprising administering to a subject having or at risk for developing a disease associated with UNC13A a therapeutically effective amount of an oligomeric compound of any of embodiments 1-45, a population of any of embodiments 46-51, an oligomeric duplex of embodiment 52 or embodiment 53, an antisense agent of any of embodiments 54-56, or a pharmaceutical composition of any of embodiments 57-59, thereby treating the disease associated with UNC13A.

Embodiment 62. The method of embodiment 61, wherein the disease associated with UNC13A is a neurodegenerative disease.

Embodiment 63. The method of embodiment 62, wherein the neurodegenerative disease is amyotrophic lateral sclerosis (ALS) or frontotemporal dementia (FTD).

Embodiment 64. The method of embodiment 63, wherein at least one symptom of the neurodegenerative disease is ameliorated.

Embodiment 65. The method of embodiment 64, wherein the at least one symptom is motor dysfunction, muscle weakness, muscle wasting, synaptic dysfunction, fatigue, difficulty speaking, difficulty swallowing, shortness of breath, cognitive impairment, decreased longevity, or a combination thereof.

Embodiment 66. The method of embodiment 65, wherein administering an oligomeric compound of any of embodiments 1-45, a population of any of embodiments 46-51, an oligomeric duplex of embodiment 52 or embodiment 53, an antisense agent of any of embodiments 54-56, or a pharmaceutical composition of any of embodiments 57-59, improves motor function, improves muscle strength, increases muscle mass, improves speaking, improves swallowing, improves breathing, improves synaptic function, improves cognition, or increases longevity.

Embodiment 67. The method of any one of embodiments 60-66, wherein the subject is a human.

Embodiment 68. A method of increasing expression of UNC13A in a cell comprising contacting the cell with an oligomeric compound of any of embodiments 1-45, a population of any of embodiments 46-51, an oligomeric duplex of embodiment 52 or embodiment 53, an antisense agent of any of embodiments 54-56, or a pharmaceutical composition of any of embodiments 57-59.

Embodiment 69. A method of decreasing the amount of UNC13A RNA containing a cryptic exon in a cell comprising contacting the cell with an oligomeric compound of any of embodiments 1-45, a population of any of embodiments 46-51, an oligomeric duplex of embodiment 52 or embodiment 53, an antisense agent of any of embodiments 54-56, or a pharmaceutical composition of any of embodiments 57-59.

Embodiment 70. The method of embodiment 69, wherein the cryptic exon is between exons 20 and 21 of UNC13A.

Embodiment 71. The method of embodiment 70, wherein the cryptic exon is selected from CE-1, CE-2, and CE-3.

Embodiment 72. The method of any of embodiments 68-71, wherein the cell is a neuron or a glial cell, optionally wherein the cell is an astrocyte or microglial cell.

Embodiment 73. The method of any of embodiments 68-72, wherein the cell is a human cell.

Embodiment 74. Use of an oligomeric compound of any of embodiments 1-45, a population of any of embodiments 46-51, an oligomeric duplex of embodiment 52 or embodiment 53, an antisense agent of any of embodiments 54-56, or a pharmaceutical composition of any of embodiments 57-59 for treating a disease associated with UNC13A.

Embodiment 75. Use of an oligomeric compound of any of embodiments 1-45, a population of any of embodiments 46-51, an oligomeric duplex of embodiment 52 or embodiment 53, an antisense agent of any of embodiments 54-56, or a pharmaceutical composition of any of embodiments 57-59 in the manufacture of a medicament for treating a disease associated with UNC13A.

Embodiment 76. The use of embodiment 74 or embodiment 75, wherein the disease associated with UNC13A is a neurodegenerative disease.

Embodiment 77. The use of embodiment 76, wherein the neurodegenerative disease is amyotrophic lateral sclerosis (ALS) or frontotemporal dementia (FTD).

### Certain Oligomeric Agents and Oligomeric Compounds

Certain embodiments provide oligomeric agents targeted to a UNC13A nucleic acid. In certain embodiments, the UNC13A nucleic acid has the sequence set forth in SEQ ID NO: 1 (complement of GENBANK Accession No. NC_000019.10, truncated from nucleosides 17598001 to 17691000), or SEQ ID NO: 2 (GENBANK Accession No. NM_001080421.2) each of which is incorporated by reference in its entirety. In certain embodiments, the oligomeric agent is a single-stranded oligomeric compound. In certain embodiments, the oligomeric agent is an oligomeric duplex.

Certain embodiments provide an oligomeric compound comprising a modified oligonucleotide consisting of 8 to 80 linked nucleosides, wherein the nucleobase sequence of the modified oligonucleotide is at least 80% complementary to an equal length portion of a UNC13A nucleic acid, and wherein the modified oligonucleotide has at least one modification selected from a modified sugar moiety and a modified internucleoside linkage. In certain embodiments, the UNC13A nucleic acid has the nucleobase sequence of SEQ ID NOs: 1 or 2. In certain embodiments, the nucleobase sequence of the modified oligonucleotide is at least 85%, at least 90%, at least 95%, or 100% complementary to an equal length portion of the UNC13A nucleic acid.

In certain embodiments, the nucleobase sequence of the modified oligonucleotide is at least 80% complementary to an equal length portion within nucleobases 48104-48121, 48106-48123, 48108-48125, 48110-48127, 48112-48129, 48114-48131, 48116-48133, 48118-48135, 48120-48137, 48122-48139, 48124-48141, 48126-48143, 48128-48145, 48130-48147, 48132-48149, 48134-48151, 48136-48153, 48138-48155, 48140-48157, 48142-48159, 48144-48161, 48146-48163, 48148-48165, 48150-48167, 48152-48169, 48154-48171, 48156-48173, 48158-48175, 48160-48177, 48162-48179, 48164-48181, 48166-48183, 48168-48185, 48170-48187, 48172-48189, 48174-48191, 48176-48193, 48178-48195, 48180-48197, 48182-48199, 48184-48201, 48186-48203, 48188-48205, 48190-48207, 48192-48209, 48194-48211, 48196-48213, 48198-48215, 48200-48217, 48202-48219, 48204-48221, 48206-48223, 48208-48225, 48210-48227, 48212-48229, 48214-48231, 48216-48233, 48218-48235, 48220-48237, 48222-48239, 48224-48241, 48226-48243, 48228-48245, 48230-48247, 48232-48249, 48234-48251, 48236-48253, 48238-48255, 48240-48257, 48242-48259, 48244-48261, 48246-48263, 48248-48265, 48250-48267, 48252-48269, 48254-48271, 48256-48273, 48258-48275, 48260-48277, 48262-48279, 48264-48281, 48266-48283, 48268-48285, 48270-48287, 48272-48289, 48274-48291, 48276-48293, 48278-48295, 48280-48297, 48282-48299, 48284-48301, 48286-48303, 48288-48305, 48290-48307, 48292-48309, 48294-48311, 48296-48313, 48298-48315, 48300-48317, 48302-48319, 48304-48321, 48306-48323, 48308-48325, 48310-48327, 48312-48329, 48314-48331, 48316-48333, 48318-48335, 48320-48337, 48322-48339, 48324-48341, 48326-48343, 48328-48345, 48330-48347, 48332-48349, 48334-48351, 48336-48353, 48338-48355, 48340-48357, 48342-48359, 48344-48361, 48346-48363, 48348-48365, 48350-48367, 48352-48369, 48354-48371, 48356-48373, 48358-48375, 48360-48377, 48362-48379, 48364-48381, 48366-48383, 48368-48385, 48370-48387, 48372-48389, 48374-48391, 48376-48393, 48378-48395, 48380-48397, 48382-48399, 48384-48401, 48385-48402, 48386-48403, 48387-48404, 48388-48405, 48389-48406, 48390-48407, 48391-48408, 48392-48409, 48393-48410, 48394-48411, 48395-48412, 48396-48413, 48397-48414, 48398-48415, 48399-48416, 48400-48417, 48401-48418, 48402-48419, 48403-48420, 48404-48421, 48405-48422, 48406-48423, 48407-48424, 48408-48425, 48409-48426, 48410-48427, 48411-48428, 48412-48429, 48413-48430, 48414-48431, 48415-48432, 48416-48433, 48417-48434, 48418-48435, 48419-48436, 48420-48437, 48421-48438, 48422-48439, 48423-48440, 48424-48441, 48425-48442, 48426-48443, 48427-48444, 48428-48445, 48429-48446, 48430-48447, 48431-48448, 48432-48449, 48433-48450, 48434-48451, 48435-48452, 48436-48453, 48437-48454, 48438-48455, 48439-48456, 48440-48457, 48441-48458, 48442-48459, 48443-48460, 48444-48461, 48445-48462, 48446-48463, 48447-48464, 48448-48465, 48449-48466, 48450-48467, 48451-48468, 48452-48469, 48453-48470, 48454-48471, 48455-48472, 48456-48473, 48457-48474, 48458-48475, 48459-48476, 48460-48477, 48461-48478, 48462-48479, 48463-48480, 48464-48481, 48465-48482, 48466-48483, 48467-48484, 48468-48485, 48469-48486, 48470-48487, 48471-48488, 48472-48489, 48473-48490, 48474-48491, 48475-48492, 48476-48493, 48477-48494, 48478-48495, 48479-48496, 48480-48497, 48481-48498, 48482-48499, 48483-48500, 48484-48501, 48486-48503, 48488-48505, 48490-48507, 48492-48509, 48494-48511, 48496-48513, 48498-48515, 48500-48517, 48502-48519, 48504-48521, 48506-48523, 48508-48525, 48510-48527, 48512-48529, 48514-48531, 48516-48533, 48518-48535, 48520-48537, 48522-48539, 48524-48541, 48526-48543, 48528-48545, 48530-48547, 48532-48549, 48534-48551, 48536-48553, 48538-48555, 48540-48557, 48542-48559, 48544-48561, 48546-48563, 48548-48565, 48550-48567, 48552-48569, 48554-48571, 48556-48573, 48558-48575, 48560-48577, 48562-48579, 48564-48581, 48566-48583, 48568-48585, 48570-48587, 48572-48589, 48574-48591, 48576-48593, 48578-48595, 48580-48597, 48582-48599, 48584-48601, 48586-48603, 48588-48605, 48590-48607, 48592-48609, 48594-48611, 48596-48613, 48598-48615, 48600-48617, 48602-48619, 48604-48621, 48606-48623, 48608-48625, 48610-48627, 48612-48629, 48614-48631, 48616-48633, 48618-48635, 48620-48637, 48622-48639, 48624-48641, 48626-48643 of SEQ ID NO: 1. In certain embodiments, the nucleobase sequence of the modified oligonucleotide is at least 85%, at least 90%, at least 95%, or 100% complementary to the nucleobase sequence of an equal length portion of the UNC13A nucleic acid.

Certain embodiments provide an oligomeric compound comprising a modified oligonucleotide consisting of 8 to 80 linked nucleosides, wherein the nucleobase sequence of the modified oligonucleotide comprises at least 8, at least 9, at least 10, at least 11, at least 12, at least 13, at least 14, at least 15, at least 16, at least 17, or 18 contiguous nucleobases of any of the nucleobase sequences of SEQ ID NOs: 21-332.

Certain embodiments provide an oligomeric compound comprising a modified oligonucleotide consisting of 18 to 80 linked nucleosides, wherein the nucleobase sequence of the modified oligonucleotide comprises the nucleobase sequence of any of nucleobase sequences of SEQ ID NOs: 21-332.

Certain embodiments provide an oligomeric compound comprising a modified oligonucleotide consisting of 18 to 80 linked nucleosides, wherein the modified oligonucleotide has a nucleobase sequence consisting of the nucleobase sequence of any of the nucleobase sequences of SEQ ID NOs: 21-332.

In any of the oligomeric compounds provided herein, the nucleobase sequence of the modified oligonucleotide can be at least 85%, at least 90%, at least 95%, or 100% complementary to an equal length portion of a UNC13A nucleic acid, wherein the UNC13A nucleic acid has the nucleobase sequence of SEQ ID NOs: 1 or 2.

In any of the oligomeric compounds provided herein, the modified oligonucleotide can consist of 10 to 25, 10 to 30, 10 to 50, 12 to 20, 12 to 25, 12 to 30, 12 to 50, 13 to 20, 13 to 25, 13 to 30, 13 to 50, 14 to 20, 14 to 25, 14 to 30, 14 to 50, 15 to 20, 15 to 25, 15 to 30, 15 to 50, 16 to 18, 16 to 20, 16 to 25, 16 to 30, 16 to 50, 17 to 20, 17 to 25, 17 to 30, 17 to 50, 18 to 20, 18 to 25, 18 to 30, 18 to 50, 19 to 20, 19 to 25, 19 to 30, 19 to 50, 20 to 25, 20 to 30, 20 to 50, 21 to 25, 21 to 30, 21 to 50, 22 to 25, 22 to 30, 22 to 50, 23 to 25, 23 to 30, or 23 to 50 linked nucleosides.

In certain embodiments, the modified oligonucleotide comprises at least 10, at least 11, at least 12, at least 13, at least 14, at least 15, at least 16, at least 17, at least 18, or at least 19 but no more than 50 linked nucleotides. In certain embodiments, the modified oligonucleotide consists of 16, 17, 18, 19, or 20 linked nucleosides.

In any of the oligomeric compounds provided herein, at least one nucleoside of the modified oligonucleotide can comprise a modified sugar moiety. In certain embodiments, the modified sugar moiety comprises a bicyclic sugar moiety, such as a 2′-4′ bridge selected from —O—CH2-; and —O—CH(CH3)—. In certain embodiments, the modified sugar moiety comprises a non-bicyclic modified sugar moiety, such as a 2′-MOE sugar moiety or 2′-OMe sugar moiety. In certain embodiments, the modified sugar moiety comprises a 2′-O—N-alkyl acetamide sugar moiety, such as a 2′-O—N-methyl acetamide sugar moiety,

In any of the oligomeric compounds provided herein, at least one nucleoside of the modified oligonucleotide compound can comprise a sugar surrogate.

In any of the oligomeric compounds provided herein, at least one internucleoside linkage of the modified oligonucleotide can comprise a modified internucleoside linkage, such as a phosphorothioate internucleoside linkage. In certain embodiments, each internucleoside linkage of the modified oligonucleotide can be a modified internucleoside linkage or each internucleoside linkage of the modified oligonucleotide can be a phosphorothioate internucleoside linkage. In certain embodiments, at least one internucleoside linkage of the modified oligonucleotide can be a phosphodiester internucleoside linkage. In certain embodiments, each internucleoside linkage of the modified oligonucleotide can be independently selected from a phosphodiester or a phosphorothioate internucleoside linkage. In certain embodiments, at least 2, at least 3, at least 4, at least 5, or at least 6 internucleoside linkages of the modified oligonucleotide can be phosphodiester internucleoside linkages. In certain embodiments, at least 10, at least 11, at least 12, at least 13, at least 14, at least 15, at least 16, at least 17, or at least 18 internucleoside linkages of the modified oligonucleotide can be phosphorothioate internucleoside linkages.

In any of the oligomeric compounds provided herein, at least one nucleobase of the modified oligonucleotide can be a modified nucleobase, such as 5-methylcytosine. In certain embodiments, each cytosine is 5-methylcytosine.

### Certain Oligomeric Duplexes

Certain embodiments are directed to oligomeric duplexes comprising a first oligomeric compound and a second oligomeric compound.

In certain embodiments, an oligomeric duplex comprises:
a first oligomeric compound comprising a first modified oligonucleotide consisting of 8 to 80 linked nucleosides, wherein the nucleobase sequence of the first modified oligonucleotide is at least 80% complementary to an equal length portion within nucleobases 48104-48121, 48106-48123, 48108-48125, 48110-48127, 48112-48129, 48114-48131,48116-48133, 48118-48135,48120-48137, 48122-48139,48124-48141, 48126-48143,48128-48145, 48130-48147, 48132-48149, 48134-48151, 48136-48153, 48138-48155, 48140-48157, 48142-48159, 48144-48161, 48146-48163, 48148-48165, 48150-48167, 48152-48169, 48154-48171, 48156-48173, 48158-48175, 48160-48177, 48162-48179, 48164-48181, 48166-48183, 48168-48185, 48170-48187, 48172-48189, 48174-48191, 48176-48193, 48178-48195, 48180-48197, 48182-48199, 48184-48201, 48186-48203, 48188-48205, 48190-48207, 48192-48209, 48194-48211, 48196-48213, 48198-48215, 48200-48217, 48202-48219, 48204-48221, 48206-48223, 48208-48225, 48210-48227, 48212-48229, 48214-48231, 48216-48233, 48218-48235, 48220-48237, 48222-48239, 48224-48241, 48226-48243, 48228-48245, 48230-48247, 48232-48249, 48234-48251, 48236-48253, 48238-48255, 48240-48257, 48242-48259, 48244-48261, 48246-48263, 48248-48265, 48250-48267, 48252-48269, 48254-48271, 48256-48273, 48258-48275, 48260-48277, 48262-48279, 48264-48281, 48266-48283, 48268-48285, 48270-48287, 48272-48289, 48274-48291, 48276-48293, 48278-48295, 48280-48297, 48282-48299, 48284-48301, 48286-48303, 48288-48305, 48290-48307, 48292-48309, 48294-48311, 48296-48313, 48298-48315, 48300-48317, 48302-48319, 48304-48321, 48306-48323, 48308-48325, 48310-48327, 48312-48329, 48314-48331, 48316-48333, 48318-48335, 48320-48337, 48322-48339, 48324-48341, 48326-48343, 48328-48345, 48330-48347, 48332-48349, 48334-48351, 48336-48353, 48338-48355, 48340-48357, 48342-48359, 48344-48361, 48346-48363, 48348-48365, 48350-48367, 48352-48369, 48354-48371, 48356-48373, 48358-48375, 48360-48377, 48362-48379, 48364-48381, 48366-48383, 48368-48385, 48370-48387, 48372-48389, 48374-48391, 48376-48393, 48378-48395, 48380-48397, 48382-48399, 48384-48401, 48385-48402, 48386-48403, 48387-48404, 48388-48405, 48389-48406, 48390-48407, 48391-48408, 48392-48409, 48393-48410, 48394-48411, 48395-48412, 48396-48413, 48397-48414, 48398-48415, 48399-48416, 48400-48417, 48401-48418, 48402-48419, 48403-48420, 48404-48421, 48405-48422, 48406-48423, 48407-48424, 48408-48425, 48409-48426, 48410-48427, 48411-48428, 48412-48429, 48413-48430, 48414-48431, 48415-48432, 48416-48433, 48417-48434, 48418-48435, 48419-48436, 48420-48437, 48421-48438, 48422-48439, 48423-48440, 48424-48441, 48425-48442, 48426-48443, 48427-48444, 48428-48445, 48429-48446, 48430-48447, 48431-48448, 48432-48449, 48433-48450, 48434-48451, 48435-48452, 48436-48453, 48437-48454, 48438-48455, 48439-48456, 48440-48457, 48441-48458, 48442-48459, 48443-48460, 48444-48461, 48445-48462, 48446-48463, 48447-48464, 48448-48465, 48449-48466, 48450-48467, 48451-48468, 48452-48469, 48453-48470, 48454-48471, 48455-48472, 48456-48473, 48457-48474, 48458-48475, 48459-48476, 48460-48477, 48461-48478, 48462-48479, 48463-48480, 48464-48481, 48465-48482, 48466-48483, 48467-48484, 48468-48485, 48469-48486, 48470-48487, 48471-48488, 48472-48489, 48473-48490, 48474-48491, 48475-48492, 48476-48493, 48477-48494, 48478-48495, 48479-48496, 48480-48497, 48481-48498, 48482-48499, 48483-48500, 48484-48501, 48486-48503, 48488-48505, 48490-48507, 48492-48509, 48494-48511, 48496-48513, 48498-48515, 48500-48517, 48502-48519, 48504-48521, 48506-48523, 48508-48525, 48510-48527, 48512-48529, 48514-48531, 48516-48533, 48518-48535, 48520-48537, 48522-48539, 48524-48541, 48526-48543, 48528-48545, 48530-48547, 48532-48549, 48534-48551, 48536-48553, 48538-48555, 48540-48557, 48542-48559, 48544-48561, 48546-48563, 48548-48565, 48550-48567, 48552-48569, 48554-48571, 48556-48573, 48558-48575, 48560-48577, 48562-48579, 48564-48581, 48566-48583, 48568-48585, 48570-48587, 48572-48589, 48574-48591, 48576-48593, 48578-48595, 48580-48597, 48582-48599, 48584-48601, 48586-48603, 48588-48605, 48590-48607, 48592-48609, 48594-48611, 48596-48613, 48598-48615, 48600-48617, 48602-48619, 48604-48621, 48606-48623, 48608-48625, 48610-48627, 48612-48629, 48614-48631, 48616-48633, 48618-48635, 48620-48637, 48622-48639, 48624-48641, 48626-48643 of SEQ ID NO: 1; and
a second oligomeric compound comprising a second modified oligonucleotide consisting of 8 to 80 linked nucleosides wherein the nucleobase sequence of the second modified oligonucleotide comprises a complementary region of at least 8 nucleobases that is at least 90% complementary to an equal length portion of the first modified oligonucleotide. In certain embodiments, the nucleobase sequence of the first modified oligonucleotide is at least 85%, at least 90%, at least 95%, or 100% complementary to an equal length portion of the UNC13A nucleic acid.

In certain embodiments, an oligomeric duplex comprises:
a first oligomeric compound comprising a first modified oligonucleotide consisting of 8 to 80 linked nucleosides wherein the nucleobase sequence of the first modified oligonucleotide comprises at least 8, at least 9, at least 10, at least 11, at least 12, at least 13, at least 14, at least 15, at least 16, at least 17, or 18 contiguous nucleobases of the nucleobase sequence of any of SEQ ID NOs 21-332, wherein each thymine is replaced by uracil; and
a second oligomeric compound comprising a second modified oligonucleotide consisting of 8 to 80 linked nucleosides wherein the nucleobase sequence of the second modified oligonucleotide comprises a complementary region of at least 8 nucleobases that is at least 90% complementary to an equal length portion of the first modified oligonucleotide. In certain embodiments, the nucleobase sequence of the first modified oligonucleotide is at least 85%, at least 90%, at least 95%, or 100% complementary to an equal length portion of the UNC13A nucleic acid.

In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide.

In certain embodiments, an oligomeric duplex comprises:
a first oligomeric compound comprising a first modified oligonucleotide consisting of 18 to 80 linked nucleosides wherein the nucleobase sequence of the first modified oligonucleotide comprises the nucleobase sequence of any of SEQ ID NOs 21-332, wherein each thymine is replaced by uracil; and
a second oligomeric compound comprising a second modified oligonucleotide consisting of 18 to 80 linked nucleosides wherein the nucleobase sequence of the second modified oligonucleotide comprises a complementary region of at least 16 nucleobases that is at least 90% complementary to an equal length portion of the first modified oligonucleotide.

In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide.

In any of the oligomeric duplexes described herein, at least one nucleoside of the first modified oligonucleotide and/or the second modified oligonucleotide can comprise a modified sugar moiety. Examples of suitable modified sugar moieties include, but are not limited to, a bicyclic sugar moiety, such as a 2′-4′ bridge selected from —O—CH2-; and —O—CH(CH3)—, and a non-bicyclic sugar moiety, such as a 2′-MOE sugar moiety, a 2′-F sugar moiety, a 2′-OMe sugar moiety, or a 2′-NMA sugar moiety. In certain embodiments, at least 80%, at least 90%, or 100% of the nucleosides of the first modified oligonucleotide and/or the second modified oligonucleotide comprises a modified sugar moiety selected from 2′-F and 2′-OMe.

In any of the oligomeric duplexes described herein, at least one nucleoside of the first modified oligonucleotide and/or the second modified oligonucleotide can comprise a sugar surrogate. Examples of suitable sugar surrogates include, but are not limited to, morpholino, peptide nucleic acid (PNA), glycol nucleic acid (GNA), and unlocked nucleic acid (UNA). In certain embodiments, at least one nucleoside of the first modified oligonucleotide comprises a sugar surrogate, which can be a GNA.

In any of the oligomeric duplexes described herein, at least one internucleoside linkage of the first modified oligonucleotide and/or the second modified oligonucleotide can comprise a modified internucleoside linkage. In certain embodiments, the modified internucleoside linkage is a phosphorothioate internucleoside linkage. In certain embodiments, at least one of the first, second, or third internucleoside linkages from the 5′ end and/or the 3′ end of the first modified oligonucleotide comprises a phosphorothioate linkage. In certain embodiments, at least one of the first, second, or third internucleoside linkages from the 5′ end and/or the 3′ end of the second modified oligonucleotide comprises a phosphorothioate linkage.

In any of the oligomeric duplexes described herein, at least one internucleoside linkage of the first modified oligonucleotide and/or the second modified oligonucleotide can comprise a phosphodiester internucleoside linkage.

In any of the oligomeric duplexes described herein, each internucleoside linkage of the first modified oligonucleotide and/or the second modified oligonucleotide can be independently selected from a phosphodiester or a phosphorothioate internucleoside linkage.

In any of the oligomeric duplexes described herein, at least one nucleobase of the first modified oligonucleotide and/or the second modified oligonucleotide can be modified nucleobase. In certain embodiments, the modified nucleobase is 5-methylcytosine.

In any of the oligomeric duplexes described herein, the first modified oligonucleotide can comprise a stabilized phosphate group attached to the 5′ position of the 5′-most nucleoside. In certain embodiments, the stabilized phosphate group comprises a cyclopropyl phosphonate or an (E)-vinyl phosphonate.

In any of the oligomeric duplexes described herein, the first modified oligonucleotide can comprise a conjugate group. In certain embodiments, the conjugate group comprises a conjugate linker and a conjugate moiety. In certain embodiments, the conjugate group is attached to the first modified oligonucleotide at the 5′-end of the first modified oligonucleotide. In certain embodiments, the conjugate group is attached to the first modified oligonucleotide at the 3′-end of the modified oligonucleotide. In certain embodiments, the conjugate group comprises N-acetyl galactosamine. In certain embodiments, the conjugate group comprises a cell-targeting moiety having an affinity for transferrin receptor (TfR), also known as TfR1 and CD71. In certain embodiments, the conjugate group comprises an anti-TfR1 antibody or fragment thereof. In certain embodiments, the conjugate group comprises a protein or peptide capable of binding TfR1. In certain embodiments, the conjugate group comprises an aptamer capable of binding TfR1. In certain embodiments, the conjugate group may comprise a conjugate moiety selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl. In certain embodiments, the conjugate group may comprise a conjugate moiety selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, or C5 alkyl, where the alkyl chain has one or more unsaturated bonds.

In any of the oligomeric duplexes described herein, the second modified oligonucleotide can comprise a conjugate group. In certain embodiments, the conjugate group comprises a conjugate linker and a conjugate moiety. In certain embodiments, the conjugate group is attached to the second modified oligonucleotide at the 5′-end of the second modified oligonucleotide. In certain embodiments, the conjugate group is attached to the second modified oligonucleotide at the 3′-end of the modified oligonucleotide. In certain embodiments, the conjugate group comprises N-acetyl galactosamine. In certain embodiments, the conjugate group comprises a cell-targeting moiety having an affinity for transferrin receptor (TfR), also known as TfR1 and CD71. In certain embodiments, the conjugate group comprises an anti-TfR1 antibody or fragment thereof. In certain embodiments, the conjugate group comprises a protein or peptide capable of binding TfR1. In certain embodiments, the conjugate group comprises an aptamer capable of binding TfR1. In certain embodiments, the conjugate group may comprise a conjugate moiety selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl. In certain embodiments, the conjugate group may comprise a conjugate moiety selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, or C5 alkyl, where the alkyl chain has one or more unsaturated bonds.

In certain embodiments, an antisense agent comprises an antisense compound, which comprises an oligomeric compound or an oligomeric duplex described herein. In certain embodiments, an antisense agent, which can comprise an oligomeric compound or an oligomeric duplex described herein, is an RNAi agent capable of modulating the amount of UNC13A nucleic acid through the activation of RISC/Ago2.

Certain embodiments provide an oligomeric agent comprising two or more oligomeric duplexes. In certain embodiments, an oligomeric agent comprises two or more of any of the oligomeric duplexes described herein. In certain embodiments, an oligomeric agent comprises two or more of the same oligomeric duplex, which can be any of the oligomeric duplexes described herein. In certain embodiments, the two or more oligomeric duplexes are linked together.

In certain embodiments, the two or more oligomeric duplexes are covalently linked together. In certain embodiments, the second modified oligonucleotides of two or more oligomeric duplexes are covalently linked together. In certain embodiments, the second modified oligonucleotides of two or more oligomeric duplexes are covalently linked together at their 3′ ends. In certain embodiments, the two or more oligomeric duplexes are covalently linked together by a glycol linker, such as a tetraethylene glycol linker. Certain such compounds are described in, e.g., Alterman, et al., Nature Biotech., 37:844-894, 2019.

### I. Certain Oligonucleotides

In certain embodiments, provided herein are oligomeric compounds comprising oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA. That is, modified oligonucleotides comprise at least one modified nucleoside (comprising a modified sugar moiety and/or a modified nucleobase) and/or at least one modified internucleoside linkage. Certain modified nucleosides and modified internucleoside linkages suitable for use in modified oligonucleotides are described below.

### A. Certain Modified Nucleosides

Modified nucleosides comprise a modified sugar moiety or a modified nucleobase or both a modified sugar moiety and a modified nucleobase. In certain embodiments, modified nucleosides comprising the following modified sugar moieties and/or the following modified nucleobases may be incorporated into modified oligonucleotides.

### 1. Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more substituent groups none of which bridges two atoms of the furanosyl ring to form a bicyclic structure. Such non bridging substituents may be at any position of the furanosyl, including but not limited to substituents at the 2′, 3′, 4′, and/or 5′ positions. In certain embodiments one or more non-bridging substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), 2′-O(CH₂)₂OCH₃ (“MOE” or “0-methoxyethyl”) and 2′-O—N-alkyl acetamide, e.g., 2′-O—N-methyl acetamide (“NMA”), 2′-O—N-dimethyl acetamide, 2′-O—N-ethyl acetamide, or 2′-O—N-propyl acetamide. For example, see U.S. Pat. No. 6,147,200, Prakash et al., 2003, Org. Lett., 5, 403-6. A “2′-O—N-methyl acetamide nucleoside” or “2′-NMA nucleoside” is shown below:

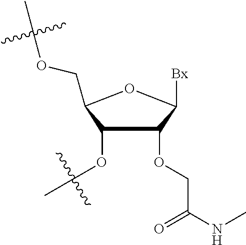

In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, 0-C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, —O(CH₂)₂ON(CH₃)₂ (“DMAOE”), 2′-O(CH₂)₂O(CH₂)₂N(CH₃)₂(“DMAEOE”), and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. In certain embodiments, non-bicyclic modified sugar moieties comprise a substituent group at the 3′-position. Examples of substituent groups suitable for the 3′-position of modified sugar moieties include but are not limited to alkoxy (e.g., methoxy), alkyl (e.g., methyl, ethyl). In certain embodiments, non-bicyclic modified sugar moieties comprise a substituent group at the 4′-position. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, ethyl, and 5′-methoxy. In certain embodiments, non-bicyclic modified sugar moieties comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836).

In certain embodiments, a 2′-substituted non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl, e.g., for example, OCH₂C(═O)—N(H)CH₃ (“NMA”).

In certain embodiments, a 2′-substituted nucleoside non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, O(CH₂)₂ON(CH₃)₂(“DMAOE”), O(CH₂)₂O(CH₂)₂N(CH₃)₂ (“DMAEOE”), and OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCH₃, and OCH₂CH₂OCH₃, and OCH2C(═O)—N(H)CH3.

In certain embodiments, modified furanosyl sugar moieties and nucleosides incorporating such modified furanosyl sugar moieties are further defined by isomeric configuration. For example, a 2′-deoxyfuranosyl sugar moiety may be in seven isomeric configurations other than the naturally occurring β-D-deoxyribosyl configuration. Such modified sugar moieties are described in, e.g., WO 2019/157531, incorporated by reference herein. A 2′-modified sugar moiety has an additional stereocenter at the 2′-position relative to a 2′-deoxyfuranosyl sugar moiety; therefore, such sugar moieties have a total of sixteen possible isomeric configurations. 2′-modified sugar moieties described herein are in the β-D-ribosyl isomeric configuration unless otherwise specified.

In certain embodiments, non-bicyclic modifed sugar moieties comprise a substituent group at the 4′-position. Examples of substituent groups suitable for the 4′-position of modified sugar moieties include but are not limited to alkoxy (e.g., methoxy), alkyl, and those described in Manoharan et al., WO2015/106128.

In certain embodiments, non-bicyclic modifed sugar moieties comprise a substituent group at the 3′-position. Examples of substituent groups suitable for the 3′-position of modified sugar moieties include, but are not limited to, alkoxy (e.g., methoxy) and alkyl (e.g., methyl, ethyl).

In certain embodiments, non-bicyclic modifed sugar moieties comprise a substituent group at the 5′-position. Examples of substituent groups suitable for the 5′-position of modified sugar moieties include, but are not limited to, vinyl, alkoxy (e.g., methoxy), and alkyl (e.g., methyl (R or S), ethyl).

In naturally occurring nucleic acids, sugars are linked to one another 3′ to 5′. In certain embodiments, oligonucleotides include one or more nucleoside or sugar moiety linked at an alternative position, for example at the 2′ position or inverted 5′ to 3′. For example, where the linkage is at the 2′ position, the 2′-substituent groups may instead be at the 3′-position.

Certain modified sugar moieties comprise a substituent that bridges two atoms of the furanosyl ring to form a second ring, resulting in a bicyclic sugar moiety. Nucleosides comprising such bicyclic sugar moieties have been referred to as bicyclic nucleosides (BNAs), locked nucleosides, or conformationally restricted nucleotides (CRN). Certain such compounds are described in US Patent Publication No. 2013/0190383; and PCT publication WO 2013/036868. In certain such embodiments, the bicyclic sugar moiety comprises a bridge between the 4′ and the 2′ furanose ring atoms. n certain such embodiments, the furanose ring is a ribose ring. Examples of such 4′ to 2′ bridging sugar substituents include but are not limited to: 4′-CH₂-2′, 4′—(CH₂)₂-2′, 4′—(CH₂)₃-2′, 4′-CH₂—O-2′ (“LNA”), 4′-CH₂—S-2′, 4′—(CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Ra)(Rb)]n-, —[C(Ra)(Rb)]n-O—, C(Ra)═C(Rb)—, C(Ra)═N—, C(═NRa)—, —C(═O)—, —C(═S)—, —O—, —Si(Ra)₂—, —S(═O)x-, and N(Ra)—;
wherein:
x is 0, 1, or 2;
n is 1, 2, 3, or 4;
each Ra and Rb is, independently, H, a protecting group, hydroxyl, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, heterocycle radical, substituted heterocycle radical, heteroaryl, substituted heteroaryl, C5-C7 alicyclic radical, substituted C5-C7 alicyclic radical, halogen, OJ1, NJ1J2, SJ1, N3, COOJ1, acyl (C(═O)—H), substituted acyl, CN, sulfonyl (S(═O)₂-J1), or sulfoxyl (S(═O)-J1); and each J1 and J2 is, independently, H, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, acyl (C(═O)—H), substituted acyl, a heterocycle radical, a substituted heterocycle radical, C1-C12 aminoalkyl, substituted C1-C12 aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129, 8362-8379; Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3, 239-243; Wengel et al., U.S. Pat. No. 7,053,207, Imanishi et al., U.S. Pat. No. 6,268,490, Imanishi et al. U.S. Pat. No. 6,770,748, Imanishi et al., U.S. RE44,779; Wengel et al., U.S. Pat. No. 6,794,499, Wengel et al., U.S. Pat. No. 6,670,461; Wengel et al., U.S. Pat. No. 7,034,133, Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191, Torsten et al., WO 2004/106356, Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; Allerson et al., US2008/0039618; and Migawa et al., US2015/0191727. In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

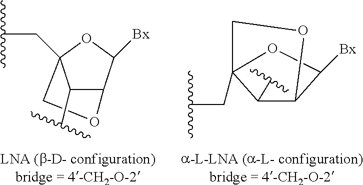

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mal Cane Ther 6(3):833-843; Grunweller, A. et al., (2003) Nucleic Acids Research 31(12):3185-3193). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, CJ. Bioorg. & Med. Chem. 2002, 10, 841-854), fluoro HNA:

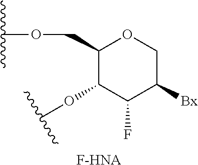

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3-fluoro tetrahydropyran), and nucleosides comprising additional modified THP compounds having the formula:

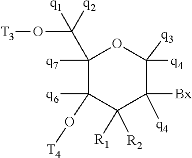

wherein, independently, for each of said modified THP nucleoside:
Bx is a nucleobase moiety;
T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a conjugate group or a 5′ or 3-terminal group;
q₁, q₂, q₃, q₄, q₅, q₆ and q₇ are each, independently, H, C1-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and
each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

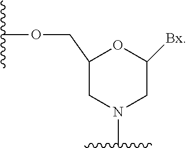

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876. In certain embodiments, sugar surrogates comprise acyclic moieties. Examples of nucleosides and oligonucleotides comprising such acyclic sugar surrogates include, but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., US2013/130378. Representative U.S. patents that teach the preparation of PNA compounds include, but are not limited to, U.S. Pat. Nos. 5,539,082; 5,714,331; and 5,719,262. Additional PNA compounds suitable for use in the oligonucleotides of the invention are described in, for example, in Nielsen et al., Science, 1991, 254, 1497-1500.

In certain embodiments, sugar surrogates are the “unlocked” sugar structure of UNA (unlocked nucleic acid) nucleosides. UNA is an unlocked acyclic nucleic acid, wherein any of the bonds of the sugar has been removed, forming an unlocked sugar surrogate. Representative U.S. publications that teach the preparation of UNA include, but are not limited to, U.S. Pat. No. 8,314,227; and US Patent Publication Nos. 2013/0096289; 2013/0011922; and 2011/0313020, the entire contents of each of which are hereby incorporated herein by reference.

In certain embodiments, sugar surrogates are the glycerol as found in GNA (glycol nucleic acid) nucleosides as depicted below:

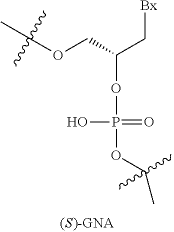

where Bx represents any nucleobase.

Many other bicyclic and tricyclic sugar and sugar surrogates are known in the art that can be used in modified nucleosides.

### 2. Certain Modified Nucleobases

In certain embodiments, modified oligonucleotides comprise one or more nucleosides comprising an unmodified nucleobase. In certain embodiments, modified oligonucleotides comprise one or more nucleosides comprising a modified nucleobase. In certain embodiments, modified oligonucleotides comprise one or more nucleosides that does not comprise a nucleobase, referred to as an abasic nucleoside. In certain embodiments, modified oligonucleotides comprise one or more inosine nucleosides (i.e., nucleosides comprising a hypoxanthine nucleobase).

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 5-methylcytosine, 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., U.S. Pat. No. 6,166,199; and Matteucci et al., U.S. Pat. No. 6,005,096.

### 3. Certain Modified Internucleoside Linkages

The naturally occurring internucleoside linkage of RNA and DNA is a 3′ to 5′ phosphodiester linkage. In certain embodiments, nucleosides of modified oligonucleotides may be linked together using one or more modified internucleoside linkages. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Methods of preparation of phosphorous-containing and non-phosphorous-containing internucleoside linkages are well known to those skilled in the art.

In certain embodiments, a modified internucleoside linkage is any of those described in WO/2021/030778, incorporated by reference herein. In certain embodiments, a modified internucleoside linkage comprises the formula:

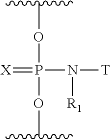

wherein independently for each internucleoside linking group of the modified oligonucleotide:
X is selected from 0 or S;
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C1-C₆ alkoxy, C1-C₆ alkyl, C1-C₆ alkenyl, C1-C₆ alkynyl, substituted C1-C₆ alkyl, substituted C1-C₆ alkenyl substituted C1-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group; and
   R₅ is selected from OCH₃, OH, C₁-C₆ alkyl, and substituted C1-C₆ alkyl.

In certain embodiments, a modified internucleoside linkage comprises a mesyl phosphoramidate linking group having a formula:

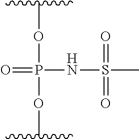

In certain embodiments, a mesyl phosphoramidate internucleoside linkage may comprise a chiral center. In certain embodiments, modified oligonucleotides comprising (Rp) and/or (Sp) mesyl phosphoramidates comprise one or more of the following formulas, respectively, wherein “B” indicates a nucleobase:

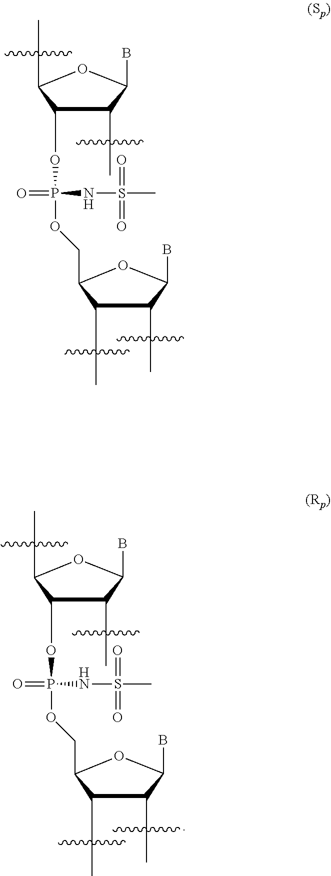

Representative internucleoside linkages having a chiral center include but are not limited to alkylphosphonates, mesyl phosphoramidates, and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate or other linkages containing chiral centers in particular stereochemical configurations. In certain embodiments, populations of modified oligonucleotides comprise phosphorothioate internucleoside linkages wherein all of the phosphorothioate internucleoside linkages are stereorandom. In certain embodiments, populations of modified oligonucleotides comprise mesyl phosphoramidate internucleoside linkages wherein all of the mesyl phosphoramidate internucleoside linkages are stereorandom. Such modified oligonucleotides can be generated using synthetic methods that result in random selection of the stereochemical configuration of each phosphorothioate or mesyl phosphoramidate linkage. Nonetheless, each individual phosphorothioate or mesyl phosphoramidate of each individual oligonucleotide molecule has a defined stereoconfiguration. In certain embodiments, populations of modified oligonucleotides are enriched for modified oligonucleotides comprising one or more particular phosphorothioate or mesyl phosphoramidate internucleoside linkages in a particular, independently selected stereochemical configuration. In certain embodiments, the particular configuration of the particular phosphorothioate or mesyl phosphoramidate linkage is present in at least 65% of the molecules in the population. In certain embodiments, the particular configuration of the particular phosphorothioate or mesyl phosphoramidate linkage is present in at least 70% of the molecules in the population. In certain embodiments, the particular configuration of the particular phosphorothioate or mesyl phosphoramidate linkage is present in at least 80% of the molecules in the population. In certain embodiments, the particular configuration of the particular phosphorothioate or mesyl phosphoramidate linkage is present in at least 90% of the molecules in the population. In certain embodiments, the particular configuration of the particular phosphorothioate or mesyl phosphoramidate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate or mesyl phosphoramidate in the (Sp) configuration. In certain embodiments, a population of modified oligonucleotides is enriched for modified oligonucleotides having at least one phosphorothioate or mesyl phosphoramidate in the (Rp) configuration. In certain embodiments, modified oligonucleotides comprising (Rp) and/or (Sp) phosphorothioates comprise one or more of the following formulas, respectively, wherein “B” indicates a nucleobase:

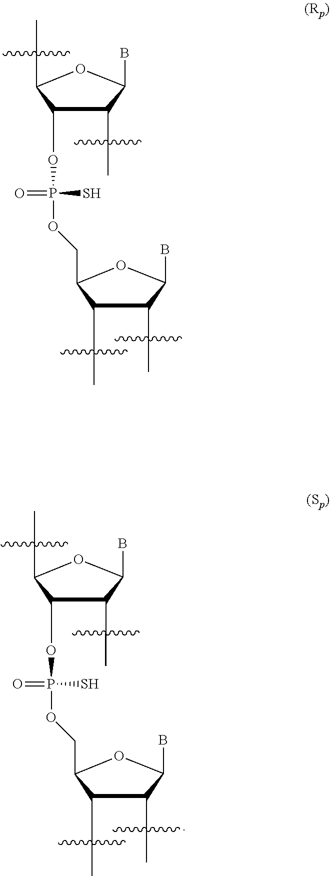

Unless otherwise indicated, chiral internucleoside linkages of modified oligonucleotides described herein can be stereorandom or in a particular stereochemical configuration.

Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl (MOP), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

In certain embodiments, modified oligonucleotides comprise one or more inverted nucleoside, as shown below:

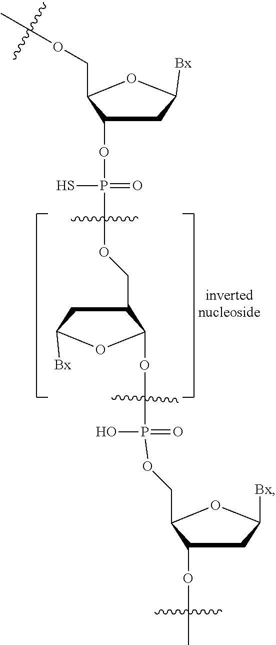

wherein each Bx independently represents any nucleobase.

In certain embodiments, an inverted nucleoside is terminal (i.e., the last nucleoside on one end of an oligonucleotide) and so only one internucleoside linkage depicted above will be present. In certain such embodiments, additional features (such as a conjugate group) may be attached to the inverted nucleoside. Such terminal inverted nucleosides can be attached to either or both ends of an oligonucleotide.

In certain embodiments, such groups lack a nucleobase and are referred to herein as inverted sugar moieties. In certain embodiments, an inverted sugar moiety is terminal (i.e., attached to the last nucleoside on one end of an oligonucleotide) and so only one internucleoside linkage above will be present. In certain such embodiments, additional features (such as a conjugate group) may be attached to the inverted sugar moiety. Such terminal inverted sugar moieties can be attached to either or both ends of an oligonucleotide.

In certain embodiments, nucleic acids can be linked 2′ to 5′ rather than the standard 3′ to 5′ linkage. Such a linkage is illustrated below.

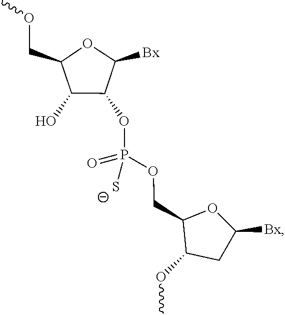

wherein each Bx represents any nucleobase.

### B. Certain Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

### 1. Certain Sugar Motifs

In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, modified oligonucleotides comprise at least 12, at least 13, at least 14, at least 15, at least 16, at least 17, at least 18, at least 19, or at least 20 nucleosides comprising a modified sugar moiety. In certain embodiments, the modified sugar moiety is selected independently from a 2′-substituted sugar moiety, a bicyclic sugar moiety, or a sugar surrogate. In certain embodiments, the 2′-substituted sugar moiety is selected from a 2′-MOE sugar moiety, a 2′-NMA sugar moiety, a 2′-OMe sugar moiety, and a 2′-F sugar moiety. In certain embodiments, the bicyclic sugar moiety is selected from a cEt sugar moiety and an LNA sugar moiety. In certain embodiments, the sugar surrogate is selected from morpholino, modified morpholino, THP, and F-HNA.

In certain embodiments, each nucleoside of a modified oligonucleotide comprises a modified sugar moiety (“fully modified oligonucleotide”). In certain embodiments, each nucleoside of a fully modified oligonucleotide comprises a 2′-substituted sugar moiety, a bicyclic sugar moiety, or a sugar surrogate. In certain embodiments, the 2′-substituted sugar moiety is selected from a 2′-MOE sugar moiety, a 2′-NMA sugar moiety, a 2′-OMe sugar moiety, and a 2′-F sugar moiety. In certain embodiments, the bicyclic sugar moiety is selected from a cEt sugar moiety and an LNA sugar moiety. In certain embodiments, the sugar surrogate is selected from morpholino, modified morpholino, THP, and F-HNA. In certain embodiments, each nucleoside of a fully modified oligonucleotide comprises the same modified sugar moiety (“uniformly modified sugar motif”). In certain embodiments, the uniformly modified sugar motif is 7 to 20 nucleosides in length. In certain embodiments, each nucleoside of the uniformly modified sugar motif comprises a 2′-substituted sugar moiety, a bicyclic sugar moiety, or a sugar surrogate. In certain embodiments, the 2′-substituted sugar moiety is selected from a 2′-MOE sugar moiety, a 2′-NMA sugar moiety, a 2′-OMe sugar moiety, and a 2′-F sugar moiety. In certain embodiments, the bicyclic sugar moiety is selected from a cEt sugar moiety and an LNA sugar moiety. In certain embodiments, the sugar surrogate is selected from morpholino, modified morpholino, THP, and F-HNA. In certain embodiments, modified oligonucleotides having at least one fully modified sugar motif may also comprise at least 1, at least 2, at least 3, or at least 4 2′-deoxyribonucleosides.

In certain embodiments, modified oligonucleotides have a sugar motif selected from 5′ to 3′: eeeeeeeeeeeeeeeeee; wherein each “e” represents a 2′-MOE sugar moiety.

### 2. Certain Nucleobase Motifs

In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines. In certain embodiments, all of the cytosine nucleobases are 5-methylcytosines and all of the other nucleobases of the modified oligonucleotide are unmodified nucleobases.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

### 3. Certain Internucleoside Linkage Motifs

In certain embodiments, oligonucleotides comprise modified and/or unmodified internucleoside linkages arranged along the oligonucleotide or region thereof in a defined pattern or motif. In certain embodiments, each internucleoside linking group is a phosphodiester internucleoside linkage (P═O). In certain embodiments, each internucleoside linking group of a modified oligonucleotide is a phosphorothioate internucleoside linkage (P═S). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate, a (Sp) phosphorothioate, and a (Rp) phosphorothioate.

In certain embodiments, modified oligonucleotides comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, or at least 19 phosphodiester internucleoside linkages. In certain embodiments, modified oligonucleotides comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, or at least 19 phosphorothioate internucleoside linkages. In certain embodiments, modified oligonucleotides comprise at least 1, at least 2, at least 3, at least 4, or at least 5 phosphodiester internucleoside linkages and the remainder of the internucleoside linkages are phosphorothioate internucleoside linkages.

In certain embodiments, modified oligonucleotides have an internucleoside linkage motif of (5′ to 3′): sssssssssssssssss, wherein each “s” represents a phosphorothioate internucleoside linkage.

In certain embodiments, modified oligonucleotides have an internucleoside linkage motif comprising one or more mesyl phosphoramidate linking groups. In certain embodiments, one or more phosphorothioate internucleoside linkages or one or more phosphodiester internucleoside linkages of the internucleoside linkage motifs herein is substituted with a mesyl phosphoramidate linking group.

### C. Certain Lengths

It is possible to increase or decrease the length of an oligonucleotide without eliminating activity. For example, in Woolf et al. (Proc. Natl. Acad. Sci. USA 89:7305-7309, 1992), a series of oligonucleotides 13-25 nucleobases in length were tested for their ability to induce cleavage of a target RNA in an oocyte injection model. Oligonucleotides 25 nucleobases in length with 8 or 11 mismatch bases near the ends of the oligonucleotides were able to direct specific cleavage of the target RNA, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

In certain embodiments, oligonucleotides consist of 16 linked nucleosides. In certain embodiments, oligonucleotides consist of 17 linked nucleosides. In certain embodiments, oligonucleotides consist of 18 linked nucleosides. In certain embodiments, oligonucleotides consist of 19 linked nucleosides. In certain embodiments, oligonucleotides consist of 20 linked nucleosides.

### D. Certain Modified Oligonucleotides

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Unless otherwise indicated, all modifications are independent of nucleobase sequence.

### E. Certain Populations of Modified Oligonucleotides

Populations of modified oligonucleotides in which all of the modified oligonucleotides of the population have the same molecular formula can be stereorandom populations or chirally enriched populations. All of the chiral centers of all of the modified oligonucleotides are stereorandom in a stereorandom population. In a chirally enriched population, at least one particular chiral center is not stereorandom in the modified oligonucleotides of the population. In certain embodiments, the modified oligonucleotides of a chirally enriched population are enriched for β-D ribosyl sugar moieties, and all of the phosphorothioate internucleoside linkages are stereorandom. In certain embodiments, the modified oligonucleotides of a chirally enriched population are enriched for both β-D ribosyl sugar moieties and at least one, particular phosphorothioate internucleoside linkage in a particular stereochemical configuration.

### F. Nucleobase Sequence

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid. In certain such embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 50%, at least 60%, at least 70%, at least 80%, at least 85%, at least 90%, at least 95%, or 100% complementary to the second oligonucleotide or nucleic acid, such as a target nucleic acid.

### II. Certain Oligomeric Compounds

In certain embodiments, provided herein are oligomeric compounds, which consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

### A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge, and clearance.

In certain embodiments, conjugation of one or more carbohydrate moieties to a modified oligonucleotide can optimize one or more properties of the modified oligonucleotide. In certain embodiments, the carbohydrate moiety is attached to a modified subunit of the modified oligonucleotide. For example, the ribose sugar of one or more ribonucleotide subunits of a modified oligonucleotide can be replaced with another moiety, e.g. a non-carbohydrate (preferably cyclic) carrier to which is attached a carbohydrate ligand. A ribonucleotide subunit in which the ribose sugar of the subunit has been so replaced is referred to herein as a ribose replacement modification subunit (RRMS), which is a modified sugar moiety. A cyclic carrier may be a carbocyclic ring system, i.e., one or more ring atoms may be a heteroatom, e.g., nitrogen, oxygen, sulphur. The cyclic carrier may be a monocyclic ring system, or may contain two or more rings, e.g. fused rings. The cyclic carrier may be a fully saturated ring system, or it may contain one or more double bonds.

In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N. Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

In certain embodiments, the conjugate group may comprise a conjugate moiety selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl.

In certain embodiments, the conjugate group may comprise a conjugate moiety selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, or C5 alkyl, where the alkyl chain has one or more unsaturated bonds.

In certain embodiments, a conjugate group is a lipid having the following structure:

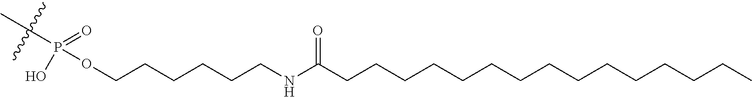 [SMILES: *P(=O)(O)OCCCCCCNC(=O)CCCCCCCCCCCCCCC]

### 1. Conjugate Moieties

Conjugate moieties include, without limitation, intercalators, reporter molecules, polyamines, polyamides, peptides, carbohydrates (e.g., GalNAc), antibodies, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

### 2. Conjugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises pyrrolidine.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate moieties to compounds, such as the oligonucleotides provided herein. In general, a bifunctional linking moiety comprises at least two functional groups. One of the functional groups is selected to react with a particular site on a compound and the other is selected to react with a conjugate moiety. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxynucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

### 3. Cell-Targeting Moieties

In certain embodiments, a conjugate group comprises a cell-targeting moiety. In certain embodiments, a conjugate group has the general formula:

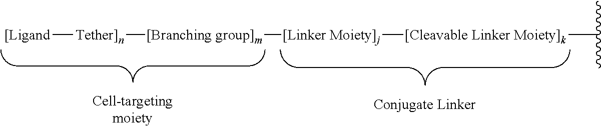

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, the cell-targeting moiety targets neurons. In certain embodiments, the cell-targeting moiety targets a neurotransmitter receptor. In certain embodiments, the cell targeting moiety targets a neurotransmitter transporter. In certain embodiments, the cell targeting moiety targets a GABA transporter. See e.g., WO 2011/131693, WO 2014/064257.

In certain embodiments, conjugate groups comprise cell-targeting moieties that have affinities for transferrin receptor (TfR) (also referred to herein as TfR1 and CD71). In certain embodiments, a conjugate group described herein comprises an anti-TfR1 antibody or fragment thereof. In certain embodiments, the conjugate group comprises a protein or peptide capable of binding TfR1. In certain embodiments, the conjugate group comprises an aptamer capable of binding TfR1. In certain embodiments, the anti-TfR1 antibody or fragment thereof can be any known in the art including but not limited to those described in WO1991/004753; WO2013/103800; WO2014/144060; WO2016/081643; WO2016/179257; WO2016/207240; WO2017/221883; WO2018/129384; WO2018/124121; WO2019/151539; WO2020/132584; WO2020/028864; U.S. Pat. Nos. 7,208,174; 9,034,329; and 10,550,188. In certain embodiments, a fragment of an anti-TfR1 antibody is F(ab′)2, Fab, Fab′, Fv, or scFv.

In certain embodiments, the conjugate group comprises a protein or peptide capable of binding TfR1. In certain embodiments, the protein or peptide capable of binding TfR1 can be any known in the art including but not limited to those described in WO2019/140050; WO2020/037150; WO2020/124032; and U.S. Pat. No. 10,138,483. In certain embodiments, the conjugate group comprises an aptamer capable of binding TfR1. In certain embodiments, the aptamer capable of binding TfR1 can be any known in the art including but not limited to those described in WO2013/163303; WO2019/033051; and WO2020/245198.

In certain embodiments, each ligand of a cell-targeting moiety has an affinity for at least one type of receptor on a target cell. In certain embodiments, each ligand has an affinity for at least one type of receptor on the surface of a mammalian liver cell. In certain embodiments, each ligand has an affinity for the hepatic asialoglycoprotein receptor (ASGP-R). In certain embodiments, each ligand is a carbohydrate.

In certain embodiments, a conjugate group comprises a cell-targeting conjugate moiety. In certain embodiments, a conjugate group has the general formula:

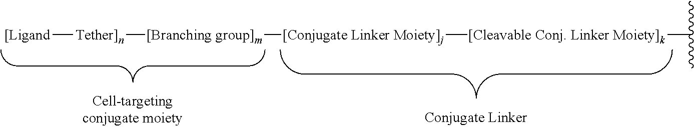

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

### B. Certain Terminal Groups

In certain embodiments, oligomeric compounds comprise one or more terminal groups. In certain such embodiments, oligomeric compounds comprise a stabilized 5′-phosphate. Stabilized 5′-phosphates include, but are not limited to 5′-phosphonates, including, but not limited to 5′-vinylphosphonates. In certain embodiments, terminal groups comprise one or more abasic sugar moieties and/or inverted nucleosides. In certain embodiments, terminal groups comprise one or more 2′-linked nucleosides or sugar moieties. In certain such embodiments, the 2′-linked group is an abasic sugar moiety.

### III. Antisense Activity

In certain embodiments, oligomeric compounds and oligomeric duplexes are capable of hybridizing to a target nucleic acid, resulting in at least one antisense activity; such oligomeric compounds and oligomeric duplexes are antisense compounds. In certain embodiments, antisense compounds have antisense activity when they modulate or increase the amount or activity of a target nucleic acid by 10%, 20%, 25%, 30%, 40%, 50% or more in the standard cell assay. In certain embodiments, antisense compounds have antisense activity when they modulate or reduce the amount of a target nucleic acid containing a cryptic exon by 10%, 20%, 25%, 30%, 40%, 50%, 60%, 70%, 80%, 90% or more in the standard cell assay. In certain embodiments, antisense compounds selectively affect one or more target nucleic acid. Such antisense compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, described herein are antisense compounds that are sufficiently “DNA-like” to elicit RNase H activity. In certain embodiments, one or more non-DNA-like nucleoside is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute. Antisense compounds that are loaded into RISC are RNAi compounds. RNAi compounds may be double-stranded (siRNA or dsRNAi) or single-stranded (ssRNA).

In certain embodiments, hybridization of an antisense compound to a target nucleic acid does not result in recruitment of a protein that cleaves that target nucleic acid. In certain embodiments, hybridization of the antisense compound to the target nucleic acid results in alteration of splicing of the target nucleic acid. In certain embodiments, hybridization of an antisense compound to a target nucleic acid results in inhibition of a binding interaction between the target nucleic acid and a protein or other nucleic acid. In certain embodiments, hybridization of an antisense compound to a target nucleic acid results in alteration of translation of the target nucleic acid. In certain embodiments, hybridization of an antisense compound to a target nucleic acid reduces the amount of RNA that includes a cryptic exon. In certain such embodiments, hybridization of an antisense compound to a target nucleic acid results in cryptic exon exclusion. In certain embodiments, hybridization of an antisense compound to a target nucleic acid increases the amount of RNA that excludes a cryptic exon. In certain embodiments, hybridization of an antisense compound to a target nucleic acid increases the amount or activity of a target nucleic acid. In certain embodiments, hybridization of an antisense compound to a target nucleic acid increases the amount of total target RNA. In certain embodiments, hybridization of an antisense compound complementary to a target nucleic acid results in alteration of splicing, leading to the exclusion of an exon in the mRNA.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein and/or a phenotypic change in a cell or animal.

### IV. Certain Target Nucleic Acids

In certain embodiments, oligomeric agents, oligomeric compounds, oligomeric duplexes, or antisense agents comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: a mature mRNA and a pre-mRNA, including intronic, exonic and untranslated regions. In certain embodiments, the target RNA is a mature mRNA. In certain embodiments, the target nucleic acid is a pre-mRNA. In certain embodiments, the target region is entirely within an intron. In certain embodiments, the target region spans an intron/exon junction. In certain embodiments, the target region is at least 50% within an intron. In certain embodiments, the target nucleic acid is the RNA transcriptional product of a retrogene. In certain embodiments, the target nucleic acid is a non-coding RNA. In certain embodiments, the target non-coding RNA is selected from: a long non-coding RNA, a short non-coding RNA, and an intronic RNA molecule.

### A. Complementarity/Mismatches to the Target Nucleic Acid and Duplex Complementarity

In certain embodiments, oligonucleotides are complementary to the target nucleic acid over the entire length of the oligonucleotide. In certain embodiments, oligonucleotides are 99%, 95%, 90%, 85%, or 80% complementary to the target nucleic acid. In certain embodiments, oligonucleotides are at least 80% complementary to the target nucleic acid over the entire length of the oligonucleotide and comprise a region that is 100% or fully complementary to a target nucleic acid. In certain embodiments, the region of full complementarity is from 6 to 20, 10 to 18, or 18 to 20 nucleobases in length.

It is possible to introduce mismatch bases without eliminating activity. For example, Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo. Maher and Dolnick (Nuc. Acid. Res. 16:3341-3358, 1988) tested a series of tandem 14 nucleobase oligonucleotides, and 28 and 42 nucleobase oligonucleotides comprised of the sequence of two or three of the tandem oligonucleotides, respectively, for their ability to arrest translation of human DHFR in a rabbit reticulocyte assay. Each of the three 14 nucleobase oligonucleotides alone was able to inhibit translation, albeit at a more modest level than the 28 or 42 nucleobase oligonucleotides.

In certain embodiments, oligonucleotides comprise one or more mismatched nucleobases relative to the target nucleic acid. In certain embodiments, antisense activity against the target is reduced by such mismatch, but activity against a non-target is reduced by a greater amount. Thus, in certain embodiments selectivity of the oligonucleotide is improved.

### B. UNC13A

In certain embodiments, oligomeric agents, oligomeric compounds, oligomeric duplexes, or antisense agents comprise or consist of an oligonucleotide comprising a region that is complementary to a target nucleic acid, wherein the target nucleic acid is a UNC13A nucleic acid. In certain embodiments, a UNC13A nucleic acid has the sequence set forth in SEQ ID NO: 1 (complement of GENBANK Accession No. NC_000019.10, truncated from nucleosides 17598001 to 17691000) or SEQ ID NO: 2 (GENBANK Accession No. NM_001080421.2). In certain embodiments, contacting a cell with an oligomeric agent, oligomeric compound, oligomeric duplex, or antisense agent complementary to SEQ ID NO: 1 or SEQ ID NO: 2 increases the amount of UNC13A RNA, and in certain embodiments increases the amount of UNC13A protein. In certain embodiments, contacting a cell with an oligomeric agent, an oligomeric compound, an oligomeric duplex, or an antisense agent complementary to SEQ ID NO: 1 or SEQ ID NO: 2 modulates splicing of UNC13A RNA. In certain embodiments, contacting a cell with an oligomeric agent, an oligomeric compound, an oligomeric duplex, or an antisense agent complementary to SEQ ID NO: 1 or SEQ ID NO: 2 reduces the amount of UNC13A RNA that includes a cryptic exon. In certain embodiments, contacting a cell with an oligomeric agent, an oligomeric compound, an oligomeric duplex, or an antisense agent complementary to SEQ ID NO: 1 or SEQ ID NO: 2 increases the amount of UNC13A RNA that excludes a cryptic exon. In certain embodiments, the cryptic exon is located in UNC13A intron 20 (i.e., the intron between exons 20 and 21 of UNC13A). In certain embodiments, the cryptic exon is CE20x (termed “CE” or “CE-1” or “cryptic exon 1”). In certain embodiments, the cryptic exon has a start site at position 48,460 and a stop site at position 48,587 of SEQ ID NO: 1. In certain embodiments, CE20x is 128 nucleobases in length, and corresponds to chromosomal co-ordinates GRCh38:19:17642413:17642541:-1 (Ma, X. R., et al., 2021, bioRxiv, doi.org/10.1101/2021.04.02.438213). In certain embodiments, CE20x has the nucleobase sequence CTGCCTGGGTTTCCTGGAAAGAACTCTTATCCCCAGGAACTAGTTTGTTGAATAAATGCTGGTGAATGAAT GAATGATTGAACAGATGAATGAGTGATGAGTAGATAAAAGGATGGATGGAGAGATGG (SEQ ID NO: 3; Ma, X. R., et al., 2021, bioRxiv, doi.org/10.1101/2021.04.02.438213, Brown, A-L., et al., 2021, bioRxiv, doi.org/10.1101/2021.04.02.438170). In certain embodiments, the cryptic exon has a start site at position 48,410 and a stop site at position 48,587 of SEQ ID NO: 1. In certain embodiments, the cryptic exon is located in UNC13A intron 20 and is 178 nucleobases in length and corresponds to chromosomal coordinates GRCh38:19:17642413: 17642591:-1 (Ma, X. R., et al., 2021, bioRxiv, doi.org/10.1101/2021.04.02.438213) (termed “CE-2” or “cryptic exon 2”). In certain embodiments, CE-2 has the nucleobase sequence CCCTAACCACTCAGGATTGGGCCGTTTGTGTCTGGGTATGTCTCTTCCAGCTGCCTGGGTTTCCTGGAAAG AACTCTTATCCCCAGGAACTAGTTTGTTGAATAAATGCTGGTGAATGAATGAATGATTGAACAGATGAATG AGTGATGAGTAGATAAAAGGATGGATGGAGAGATGG (SEQ ID NO: 4). In certain embodiments, the cryptic exon has a start site at position 48,157 and a stop site at position 48,587 of SEQ ID NO: 1. In certain embodiments, the cryptic exon is located in UNC13A intron 20 and is 431 nucleobases in length and corresponds to chromosomal coordinates GRCh38:19:17642413: 17642844:-1 (Ma, X. R., et al., 2021, bioRxiv, doi.org/10.1101/2021.04.02.438213) (termed “CE-3” or “cryptic exon 3”). In certain embodiments, CE-3 has the nucleobase sequence

(SEQ ID NO: 5)
GTGAGGGTCATTGCTCGGCCCCTCCCATGCCACTTCCACTCACCATTCCT
GCCTGCCCAGCTCTTCCTCTTTCTGGCCACACCATCCACACTCTCCTGGC
CCTCTGAGACTGCCCGCCATGCCATTCCCTTTACCTGGAAAACTCCTCCC
TATCCATCAAAGTCCAGATTCAGGGTCACCTCCTCTGGGAAGCCCACCTT
GGCCTCCAGGTTGACTCTCACTACTCATCATCAGGTTCTTCCTTCTATTC
CAGCCCTAACCACTCAGGATTGGGCCGTTTGTGTCTGGGTATGTCTCTTC
CAGCTGCCTGGGTTTCCTGGAAAGAACTCTTATCCCCAGGAACTAGTTTG
TTGAATAAATGCTGGTGAATGAATGAATGATTGAACAGATGAATGAGTGA
GTAGTAGATAAAAGGATGGATGGAGAGATGG.

In certain embodiments, the oligomeric agent, oligomeric compound, or antisense agent consists of a modified oligonucleotide. In certain embodiments, oligomeric agent, oligomeric compound, or antisense agent consists of a modified oligonucleotide and a conjugate group.

### C. Certain Target Nucleic Acids in Certain Tissues

In certain embodiments, oligomeric agents, oligomeric compounds, or antisense agents comprise or consist of an oligonucleotide comprising a region that is complementary to a target nucleic acid, wherein the target nucleic acid is expressed in a pharmacologically relevant tissue. In certain embodiments, the pharmacologically relevant tissues are the cells and tissues that comprise the central nervous system (CNS), including, but not limited to, spinal cord, cortex (including but not limited to motor, frontal, and temporal), hippocampus, medulla, and pons.

### V. Certain Hotspot Regions

### 1. Nucleobases 48,128-48,151 of SEQ ID NO: 1

In certain embodiments, nucleobases 48,128-48,151 of SEQ ID NO: 1 comprise a hotspot region. In certain embodiments, oligomeric compounds or antisense agents are complementary to a portion of nucleobases 48,128-48,151 of SEQ ID NO: 1. In certain embodiments, the oligomeric compounds or antisense agents are 18 nucleobases in length. In certain embodiments, each nucleoside of the oligomeric compound or antisense agent comprises a 2′-MOE sugar moiety. In certain embodiments, all of the internucleoside linkages of the oligomeric compound or antisense agent are phosphorothioate internucleoside linkages.

The nucleobase sequences of SEQ ID NOs: 290, 291, 293, and 294 are complementary to nucleobases 48,128-48,151 of SEQ ID NO: 1. The nucleobase sequences of the oligomeric compounds or antisense agents of compound NOs: 1616310, 1616311, 1616313, and 1616314 are complementary to nucleobases 48,128-48,151 of SEQ ID NO: 1.

In certain embodiments, oligomeric compounds or antisense agents complementary to a portion of nucleobases 48,128-48,151 of SEQ ID NO: 1 achieve at least 69% reduction of UNC13A RNA that includes CE-1 in a standard cell assay, as measured with human primer-probe set RTS54362 (designed to specifically recognize cryptic exon 1, thus measuring RNA that includes cryptic exon 1). In certain embodiments, oligomeric compounds or antisense agents complementary to a portion of nucleobases 48,128-48,151 of SEQ ID NO: 1 achieve an average of 78.8% reduction of UNC13A RNA that includes CE-1, in a standard cell assay as measured with human primer-probe set RTS54362.

### 2. Nucleobases 48,432-48,465 of SEQ ID NO: 1

In certain embodiments, nucleobases 48,432-48,465 of SEQ ID NO: 1 comprise a hotspot region. In certain embodiments, oligomeric compounds or antisense agents are complementary to a portion of nucleobases 48,432-48,465 of SEQ ID NO: 1. In certain embodiments, the oligomeric compounds or antisense agents are 18 nucleobases in length. In certain embodiments, each nucleoside of the oligomeric compound or antisense agent comprises a 2′-MOE sugar moiety. In certain embodiments, all of the internucleoside linkages of the oligomeric compound or antisense agent are phosphorothioate internucleoside linkages.

The nucleobase sequences of SEQ ID NOs: 85-101 are complementary to nucleobases 48,432-48,465 of SEQ ID NO: 1. The nucleobase sequences of the oligomeric compounds or antisense agents of compound NOs: 1616105-1616121 are complementary to nucleobases 48,432-48,465 of SEQ ID NO: 1.

In certain embodiments, oligomeric compounds or antisense agents complementary to a portion of nucleobases 48,432-48,465 of SEQ ID NO: 1 achieve at least 52% reduction of UNC13A RNA that includes CE-1, CE-2, and/or CE-3 in a standard cell assay as measured with human primer-probe set RTS54363 (designed to recognize all three cryptic exons thus measuring RNA that includes any of the three cryptic exons between exons 20 and 21 of UNC13A (complete cryptic exon inclusion)). In certain embodiments, oligomeric compounds or antisense agents complementary to a portion of nucleobases 48,432-48,465 of SEQ ID NO: 1 achieve an average of 65.4% reduction of UNC13A RNA that includes CE-1, CE-2, and/or CE-3 in a standard cell assay as measured with human primer-probe set RTS54363.

### 3. Nucleobases 48,466-48,561 of SEQ ID NO: 1

In certain embodiments, nucleobases 48,466-48,561 of SEQ ID NO: 1 comprise a hotspot region. In certain embodiments, oligomeric compounds or antisense agents are complementary to a portion of nucleobases 48,466-48,561 of SEQ ID NO: 1. In certain embodiments, the oligomeric compounds or antisense agents are 18 nucleobases in length. In certain embodiments, each nucleoside of the oligomeric compound or antisense agent comprises a 2′-MOE sugar moiety. In certain embodiments, all of the internucleoside linkages of the oligomeric compounds or antisense oligonucleotide are phosphorothioate internucleoside linkages.

The nucleobase sequences of SEQ ID NOs: 21-30, 32-41, 43-52, 54-60, 62-66, and 326-332 are complementary to nucleobases 48,466-48,561 of SEQ ID NO: 1. The nucleobase sequences of the oligomeric compounds or antisense agents of compound NOs: 1616041-1616050, 1616052-1616061, 1616063-1616072, 1616074-1616080, 1616082-1616086, and 1616346-1616352 are complementary to nucleobases 48,466-48,561 of SEQ ID NO: 1.

In certain embodiments, oligomeric compounds or antisense agents complementary to a portion of nucleobases 48,466-48,561 of SEQ ID NO: 1 achieve at least 46% reduction of UNC13A RNA that includes CE-1, CE-2, and/or CE-3 in a standard cell assay as measured with human primer-probe set RTS54363. In certain embodiments, oligomeric compounds or antisense agents complementary to a portion of nucleobases 48,466-48,561 of SEQ ID NO: 1 achieve an average of 75.9% reduction of UNC13A RNA that includes CE-1, CE-2, and/or CE-3 in a standard cell assay, as measured with human primer-probe set RTS54363.

### VI. Certain Methods and Uses

Certain embodiments provided herein relate to methods of increasing the amount or activity of UNC13A RNA or reducing the amount of UNC13A RNA containing a cryptic exon, which can be useful for treating a disease associated with UNC13A. Examples of diseases treatable with the oligomeric compounds, modified oligonucleotides, oligomeric duplexes, and antisense agents include neurodegenerative diseases. In certain embodiments, the neurodegenerative disease is amyotrophic lateral sclerosis (ALS) or frontotemporal dementia (FTD).

In certain embodiments, a method comprises administering to a subject an oligomeric compound, a modified oligonucleotide, an oligomeric duplex, or an antisense agent, any of which having a nucleobase sequence complementary to an UNC13A nucleic acid. In certain embodiments, the subject has a neurodegenerative disease. In certain embodiment, the subject has amyotrophic lateral sclerosis (ALS) or frontotemporal dementia (FTD).

In certain embodiments, a method of treating a disease associated with UNC13A comprises administering to a subject an oligomeric compound, a modified oligonucleotide, an oligomeric duplex, or an antisense agent, any of which having a nucleobase sequence complementary to a UNC13A nucleic acid. In certain embodiments, the subject has or is at risk for developing a disease associated with UNC13A. In certain embodiments, the subject has a neurodegenerative disease. In certain embodiment, the subject has amyotrophic lateral sclerosis (ALS) or frontotemporal dementia (FTD). In certain embodiments, at least one symptom of the neurodegenerative diseases is ameliorated. In certain embodiments, the symptom is motor dysfunction, muscle weakness, muscle wasting, synaptic dysfunction, fatigue, difficulty speaking, difficulty swallowing, shortness of breath, cognitive impairment, decreased longevity, or a combination thereof.

In certain embodiments, a method of increasing expression of UNC13A nucleic acid, for example RNA, in a cell comprises contacting the cell with an oligomeric compound, a modified oligonucleotide, an oligomeric duplex, or an antisense agent, any of which having a nucleobase sequence complementary to a UNC13A nucleic acid. In certain embodiments, the cell is a neuron or a glial cell. In certain embodiments, the cell is an astrocyte or microglial cell. In certain embodiments, the cell is a human cell.

In certain embodiments, a method of reducing the amount of UNC13A containing a cryptic exon, in a cell comprises contacting the cell with an oligomeric compound, a modified oligonucleotide, an oligomeric duplex, or an antisense agent, any of which having a nucleobase sequence complementary to a UNC13A nucleic acid. In certain embodiments, the cell is a neuron or a glial cell. In certain embodiments, the cell is an astrocyte or microglial cell. In certain embodiments, the cell is a human cell.

Certain embodiments are drawn to an oligomeric compound, a modified oligonucleotide, an oligomeric duplex, or an antisense agent, any of which having a nucleobase sequence complementary to a UNC13A nucleic acid, for use in treating a disease associated with UNC13A or for use in the manufacture of a medicament for treating a disease associated with UNC13A. In certain embodiments, the disease associated with UNC13A is a neurodegenerative disease. In certain embodiments, the neurodegenerative disease is amyotrophic lateral sclerosis (ALS) or frontotemporal dementia (FTD).

In any of the methods or uses described herein, the oligomeric agent, the oligomeric compound, the modified oligonucleotide, the oligomeric duplex, or the antisense agent can be any described herein.

### VI. Certain Pharmaceutical Compositions

In certain embodiments, described herein are pharmaceutical compositions comprising one or more oligomeric agents, oligomeric compounds, oligomeric duplexes, or antisense agents. In certain embodiments, the one or more oligomeric agents, oligomeric compounds, oligomeric duplexes, or antisense agents each comprises a modified oligonucleotide. In certain embodiments, the one or more oligomeric agents, oligomeric compounds, or antisense agents each consists of a modified oligonucleotide. In certain embodiments, the pharmaceutical composition comprises a pharmaceutically acceptable diluent or carrier. In certain embodiments, a pharmaceutical composition comprises, consists essentially of, or consists of a sterile saline solution and one or more oligomeric agents, oligomeric compounds, oligomeric duplexes, or antisense agents. In certain embodiments, the sterile saline is pharmaceutical grade saline. In certain embodiments, a pharmaceutical composition comprises, consists essentially of, or consists of one or more oligomeric agents, oligomeric compounds, oligomeric duplexes, or antisense agents and sterile water. In certain embodiments, the sterile water is pharmaceutical grade water. In certain embodiments, a pharmaceutical composition comprises, consists essentially of, or consists of one or more oligomeric agents, oligomeric compounds, oligomeric duplexes, or antisense agents and phosphate-buffered saline (PBS). In certain embodiments, the sterile PBS is pharmaceutical grade PBS. In certain embodiments, a pharmaceutical composition comprises, consists essentially of, or consists of one or more oligomeric agents, oligomeric compounds, oligomeric duplexes, or antisense agents and artificial cerebrospinal fluid (“artificial CSF” or “aCSF”). In certain embodiments, the artificial cerebrospinal fluid is pharmaceutical grade artificial cerebrospinal fluid.

In certain embodiments, a pharmaceutical composition comprises an oligomeric agent, an oligomeric compound, an oligomeric duplex, an antisense agent, or a modified oligonucleotide and PBS. In certain embodiments, a pharmaceutical composition consists of an oligomeric agent, an oligomeric compound, an oligomeric duplex, an antisense agent, or a modified oligonucleotide and PBS. In certain embodiments, a pharmaceutical composition consists essentially of an oligomeric agent, an oligomeric compound, an oligomeric duplex, an antisense agent, or a modified oligonucleotide and PBS. In certain embodiments, the PBS is pharmaceutical grade.

In certain embodiments, a pharmaceutical composition comprises an oligomeric agent, an oligomeric compound, an oligomeric duplex, an antisense agent, or a modified oligonucleotide and artificial cerebrospinal fluid (aCSF). In certain embodiments, a pharmaceutical composition consists of an oligomeric agent, an oligomeric compound, an oligomeric duplex, an antisense agent, or a modified oligonucleotide and artificial cerebrospinal fluid. In certain embodiments, a pharmaceutical composition consists essentially of an oligomeric agent, an oligomeric compound, an oligomeric duplex, an antisense agent, or a modified oligonucleotide and artificial cerebrospinal fluid. In certain embodiments, the artificial cerebrospinal fluid is pharmaceutical grade.

In certain embodiments, aCSF comprises sodium chloride, potassium chloride, sodium dihydrogen phosphate dihydrate, sodium phosphate dibasic anhydrous, calcium chloride dihydrate, and magnesium chloride hexahydrate. In certain embodiments, the pH of an aCSF solution is modulated with a suitable pH-adjusting agent, for example, with acids such as hydrochloric acid and alkalis such as sodium hydroxide, to a range of from about 7.1-7.3, or to about 7.2.

In certain embodiments, pharmaceutical compositions comprise one or more oligomeric agents, oligomeric compounds, oligomeric duplexes, or antisense agents and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, oligomeric agents, oligomeric compounds, oligomeric duplexes, or antisense agents may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an oligomeric agent, an oligomeric compound, an oligomeric duplex, or an antisense agent encompass any pharmaceutically acceptable salts of the oligomeric agent, the oligomeric compound, the oligomeric duplex, or the antisense agent; esters of the oligomeric agent, the oligomeric compound, the oligomeric duplex, or the antisense agent; or salts of such esters. In certain embodiments, pharmaceutical compositions comprising oligomeric agents, oligomeric compounds, oligomeric duplexes, or antisense agents comprising one or more modified oligonucleotides, upon administration to an animal, including a human, are capable of providing (directly or indirectly) the biologically active metabolite or residue thereof. Accordingly, for example, the disclosure is also drawn to pharmaceutically acceptable salts of oligomeric agents, oligomeric compounds, oligomeric duplexes, or antisense agents, prodrugs, pharmaceutically acceptable salts of such prodrugs, and other bioequivalents. In certain embodiments, pharmaceutically acceptable salts comprise inorganic salts, such as monovalent or divalent inorganic salts. Suitable pharmaceutically acceptable salts include, but are not limited to, sodium, potassium, calcium, and magnesium salts. In certain embodiments, prodrugs comprise one or more conjugate group attached to a modified oligonucleotide, wherein the conjugate group is cleaved by endogenous nucleases within the body.

In certain embodiments, oligomeric agents, oligomeric compounds, oligomeric duplexes, or antisense agents are lyophilized and isolated as sodium salts. In certain embodiments, the sodium salt of an oligomeric agent, an oligomeric compound, an oligomeric duplex, or an antisense agent is mixed with a pharmaceutically acceptable diluent. In certain embodiments, the pharmaceutically acceptable diluent comprises sterile saline, sterile water, PBS, or aCSF. In certain embodiments, the sodium salt of an oligomeric agent, an oligomeric compound, an oligomeric duplex, or an antisense agent is mixed with PBS. In certain embodiments, the sodium salt of an oligomeric agent, an oligomeric compound, an oligomeric duplex, or an antisense agent is mixed with aCSF.

Lipid moieties have been used in nucleic acid therapies in a variety of methods. In certain such methods, the nucleic acid, such as an oligomeric agent, an oligomeric compound, an oligomeric duplex, or an antisense agent is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue. In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV), etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

Under certain conditions, certain compounds disclosed herein act as acids. Although such compounds may be drawn or described in protonated (free acid) form, or ionized and in association with a cation (salt) form, aqueous solutions of such compounds exist in equilibrium among such forms. For example, a phosphate linkage of an oligonucleotide in aqueous solution exists in equilibrium among free acid, anion, and salt forms. Unless otherwise indicated, compounds described herein are intended to include all such forms. Moreover, certain oligonucleotides have several such linkages, each of which is in equilibrium. Thus, oligonucleotides in solution exist in an ensemble of forms at multiple positions all at equilibrium. The term “oligonucleotide” is intended to include all such forms. Drawn structures necessarily depict a single form. Nevertheless, unless otherwise indicated, such drawings are likewise intended to include corresponding forms. Herein, a structure depicting the free acid of a compound followed by the term “or a salt thereof” or “or a pharmaceutically acceptable salt thereof” expressly includes all such forms that may be fully or partially protonated/de-protonated/in association with a cation. In certain embodiments, one or more specific cation is identified. The cations may include, but are not limited to, sodium, potassium, calcium, or magnesium.

In certain embodiments, modified oligonucleotides, oligomeric agents, oligomeric compounds, oligomeric duplexes, or antisense agents are in aqueous solution with sodium. In certain embodiments, modified oligonucleotides, oligomeric agents, oligomeric compounds, oligomeric duplexes, or antisense agents are in aqueous solution with potassium. In certain embodiments, modified oligonucleotides, oligomeric agents, oligomeric compounds, oligomeric duplexes, or antisense agents are in aqueous solution with calcium. In certain embodiments, modified oligonucleotides, oligomeric agents, oligomeric compounds, oligomeric duplexes, or antisense agents are in aqueous solution with magnesium. In certain embodiments, modified oligonucleotides, oligomeric agents, oligomeric compounds, oligomeric duplexes, or antisense agents are in PBS. In certain embodiments, modified oligonucleotides, oligomeric agents, oligomeric compounds, oligomeric duplexes, or antisense agents are in aCSF. In certain embodiments, modified oligonucleotides, oligomeric agents, oligomeric compounds, oligomeric duplexes, or antisense agents are in water. In certain such embodiments, the pH of the solution is adjusted with NaOH and/or HCl to achieve a desired pH.

### Nonlimiting Disclosure and Incorporation by Reference

Each of the literature and patent publications listed herein is incorporated by reference in its entirety.

While certain compounds and compositions described herein have been described with specificity in accordance with certain embodiments, the following examples serve only to illustrate the compounds described herein and are not intended to limit the same. Each of the references, GenBank accession numbers, ENSEMBL identifiers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of an uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein ^{m}C indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as α or β such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms, unless specified otherwise. Likewise, tautomeric forms of the compounds herein are also included unless otherwise indicated. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

### Example 1: Design of Uniform MOE Modified Oligonucleotides with Uniform Phosphorothioate Internucleoside Linkages Complementary to a Human UNC13A Nucleic Acid

Modified oligonucleotides complementary to a human UNC13A nucleic acid were designed. The modified oligonucleotides in the table below are uniform MOE modified oligonucleotides with uniform phosphorothioate internucleoside linkages. The modified oligonucleotides are 18 nucleosides in length. The sugar motif for the modified oligonucleotides is (from 5′ to 3′): eeeeeeeeeeeeeeeeee, wherein each “e” represents a 2′-MOE sugar moiety. The internucleoside linkage motif for the modified oligonucleotides is (from 5′ to 3′): sssssssssssssssss, wherein each “s” represents a phosphorothioate internucleoside linkage. Each cytosine residue is a 5-methylcytosine. “Start site” in the table below indicates the 5′-most nucleoside to which the modified oligonucleotide is complementary in the target nucleic acid sequence. “Stop site” in the table below indicates the 3′-most nucleoside to which the modified oligonucleotide is complementary in the target nucleic acid sequence. Each modified oligonucleotide listed in the table below is 100% complementary to SEQ ID NO: 1 (complement of GENBANK Accession No. NC_000019.10, truncated from nucleosides 17598001 to 17691000), to SEQ ID NO: 2 (GENBANK Accession No. NM_001080421.2), or to both. “N/A” indicates that the modified oligonucleotide is not 100% complementary to that particular target nucleic acid sequence.

TABLE 1
Uniform MOE modified oligonucleotides
complementary to human UNC13A, with uniform
phosphorothioate internucleoside linkages

| | | SEQ | SEQ | SEQ | SEQ | |
|---|---|---|---|---|---|---|
| | | ID | ID | ID | ID | |
| | | No: | No: | No: | No: | |
| Com- | | 1 | 1 | 2 | 2 | SEQ |
| pound | Sequence | Start | Stop | Start | Stop | ID |
| No. | (5′ to 3′) | Site | Site | Site | Site | No. |
| 1616041 | TAGTTCCTGGGGATAAGA | 48484 | 48501 | N/A | N/A | 21 |
| 1616042 | AGTTCCTGGGGATAAGAG | 48483 | 48500 | N/A | N/A | 22 |
| 1616043 | GTTCCTGGGGATAAGAGT | 48482 | 48499 | N/A | N/A | 23 |
| 1616044 | TTCCTGGGGATAAGAGTT | 48481 | 48498 | N/A | N/A | 24 |
| 1616045 | TCCTGGGGATAAGAGTTC | 48480 | 48497 | N/A | N/A | 25 |
| 1616046 | CCTGGGGATAAGAGTTCT | 48479 | 48496 | N/A | N/A | 26 |
| 1616047 | CTGGGGATAAGAGTTCTT | 48478 | 48495 | N/A | N/A | 27 |
| 1616048 | TGGGGATAAGAGTTCTTT | 48477 | 48494 | N/A | N/A | 28 |
| 1616049 | GGGGATAAGAGTTCTTTC | 48476 | 48493 | N/A | N/A | 29 |
| 1616050 | GGGATAAGAGTTCTTTCC | 48475 | 48492 | N/A | N/A | 30 |
| 1616051 | AGCCACGAATCTACCCAC | 48620 | 48637 | N/A | N/A | 31 |
| 1616052 | CATCTGTTCAATCATTCA | 48530 | 48547 | N/A | N/A | 32 |
| 1616053 | TCTGTTCAATCATTCATT | 48528 | 48545 | N/A | N/A | 33 |
| 1616054 | TGTTCAATCATTCATTCA | 48526 | 48543 | N/A | N/A | 34 |
| 1616055 | TTCAATCATTCATTCATT | 48524 | 48541 | N/A | N/A | 35 |
| 1616056 | CAATCATTCATTCATTCA | 48522 | 48539 | N/A | N/A | 36 |
| 1616057 | ATCATTCATTCATTCACC | 48520 | 48537 | N/A | N/A | 37 |
| 1616058 | CATTCATTCATTCACCAG | 48518 | 48535 | N/A | N/A | 38 |
| 1616059 | TTCATTCATTCACCAGCA | 48516 | 48533 | N/A | N/A | 39 |
| 1616060 | CATTCATTCACCAGCATT | 48514 | 48531 | N/A | N/A | 40 |
| 1616061 | TTCATTCACCAGCATTTA | 48512 | 48529 | N/A | N/A | 41 |
| 1616062 | CCACGAATCTACCCACCA | 48618 | 48635 | N/A | N/A | 42 |
| 1616063 | CATTCACCAGCATTTATT | 48510 | 48527 | N/A | N/A | 43 |
| 1616064 | TTCACCAGCATTTATTCA | 48508 | 48525 | N/A | N/A | 44 |
| 1616065 | CACCAGCATTTATTCAAC | 48506 | 48523 | N/A | N/A | 45 |
| 1616066 | CCAGCATTTATTCAACAA | 48504 | 48521 | N/A | N/A | 46 |
| 1616067 | AGCATTTATTCAACAAAC | 48502 | 48519 | N/A | N/A | 47 |
| 1616068 | CATTTATTCAACAAACTA | 48500 | 48517 | N/A | N/A | 48 |
| 1616069 | TTTATTCAACAAACTAGT | 48498 | 48515 | N/A | N/A | 49 |
| 1616070 | TATTCAACAAACTAGTTC | 48496 | 48513 | N/A | N/A | 50 |
| 1616071 | TTCAACAAACTAGTTCCT | 48494 | 48511 | N/A | N/A | 51 |
| 1616072 | CAACAAACTAGTTCCTGG | 48492 | 48509 | N/A | N/A | 52 |
| 1616073 | ACGAATCTACCCACCAAC | 48616 | 48633 | N/A | N/A | 53 |
| 1616074 | ACAAACTAGTTCCTGGGG | 48490 | 48507 | N/A | N/A | 54 |
| 1616075 | AAACTAGTTCCTGGGGAT | 48488 | 48505 | N/A | N/A | 55 |
| 1616076 | ACTAGTTCCTGGGGATAA | 48486 | 48503 | N/A | N/A | 56 |
| 1616077 | GGATAAGAGTTCTTTCCA | 48474 | 48491 | N/A | N/A | 57 |
| 1616078 | GATAAGAGTTCTTTCCAG | 48473 | 48490 | N/A | N/A | 58 |
| 1616079 | ATAAGAGTTCTTTCCAGG | 48472 | 48489 | N/A | N/A | 59 |
| 1616080 | TAAGAGTTCTTTCCAGGA | 48471 | 48488 | N/A | N/A | 60 |
| 1616081 | GAATCTACCCACCAACTC | 48614 | 48631 | N/A | N/A | 61 |
| 1616082 | AAGAGTTCTTTCCAGGAA | 48470 | 48487 | N/A | N/A | 62 |
| 1616083 | AGAGTTCTTTCCAGGAAA | 48469 | 48486 | N/A | N/A | 63 |
| 1616084 | GAGTTCTTTCCAGGAAAC | 48468 | 48485 | N/A | N/A | 64 |
| 1616085 | AGTTCTTTCCAGGAAACC | 48467 | 48484 | N/A | N/A | 65 |
| 1616086 | GTTCTTTCCAGGAAACCC | 48466 | 48483 | N/A | N/A | 66 |
| 1616087 | TTCTTTCCAGGAAACCCA | 48465 | 48482 | N/A | N/A | 67 |
| 1616088 | TCTTTCCAGGAAACCCAG | 48464 | 48481 | N/A | N/A | 68 |
| 1616089 | CTTTCCAGGAAACCCAGG | 48463 | 48480 | N/A | N/A | 69 |
| 1616090 | TTTCCAGGAAACCCAGGC | 48462 | 48479 | N/A | N/A | 70 |
| 1616091 | TTCCAGGAAACCCAGGCA | 48461 | 48478 | N/A | N/A | 71 |
| 1616092 | TCCAGGAAACCCAGGCAG | 48460 | 48477 | N/A | N/A | 72 |
| 1616093 | CCAGGAAACCCAGGCAGC | 48459 | 48476 | N/A | N/A | 73 |
| 1616094 | CAGGAAACCCAGGCAGCT | 48458 | 48475 | N/A | N/A | 74 |
| 1616095 | AGGAAACCCAGGCAGCTG | 48457 | 48474 | N/A | N/A | 75 |
| 1616096 | GGAAACCCAGGCAGCTGG | 48456 | 48473 | N/A | N/A | 76 |
| 1616097 | GAAACCCAGGCAGCTGGA | 48455 | 48472 | N/A | N/A | 77 |
| 1616098 | AAACCCAGGCAGCTGGAA | 48454 | 48471 | N/A | N/A | 78 |
| 1616099 | AACCCAGGCAGCTGGAAG | 48453 | 48470 | N/A | N/A | 79 |
| 1616100 | ACCCAGGCAGCTGGAAGA | 48452 | 48469 | N/A | N/A | 80 |
| 1616101 | CCCAGGCAGCTGGAAGAG | 48451 | 48468 | N/A | N/A | 81 |
| 1616102 | ATCTACCCACCAACTCAT | 48612 | 48629 | N/A | N/A | 82 |
| 1616103 | CCAGGCAGCTGGAAGAGA | 48450 | 48467 | N/A | N/A | 83 |
| 1616104 | CAGGCAGCTGGAAGAGAC | 48449 | 48466 | N/A | N/A | 84 |
| 1616105 | AGGCAGCTGGAAGAGACA | 48448 | 48465 | N/A | N/A | 85 |
| 1616106 | GGCAGCTGGAAGAGACAT | 48447 | 48464 | N/A | N/A | 86 |
| 1616107 | GCAGCTGGAAGAGACATA | 48446 | 48463 | N/A | N/A | 87 |
| 1616108 | CAGCTGGAAGAGACATAC | 48445 | 48462 | N/A | N/A | 88 |
| 1616109 | AGCTGGAAGAGACATACC | 48444 | 48461 | N/A | N/A | 89 |
| 1616110 | GCTGGAAGAGACATACCC | 48443 | 48460 | N/A | N/A | 90 |
| 1616111 | CTGGAAGAGACATACCCA | 48442 | 48459 | N/A | N/A | 91 |
| 1616112 | TGGAAGAGACATACCCAG | 48441 | 48458 | N/A | N/A | 92 |
| 1616113 | GGAAGAGACATACCCAGA | 48440 | 48457 | N/A | N/A | 93 |
| 1616114 | GAAGAGACATACCCAGAC | 48439 | 48456 | N/A | N/A | 94 |
| 1616115 | AAGAGACATACCCAGACA | 48438 | 48455 | N/A | N/A | 95 |
| 1616116 | AGAGACATACCCAGACAC | 48437 | 48454 | N/A | N/A | 96 |
| 1616117 | GAGACATACCCAGACACA | 48436 | 48453 | N/A | N/A | 97 |
| 1616118 | AGACATACCCAGACACAA | 48435 | 48452 | N/A | N/A | 98 |
| 1616119 | GACATACCCAGACACAAA | 48434 | 48451 | N/A | N/A | 99 |
| 1616120 | ACATACCCAGACACAAAC | 48433 | 48450 | N/A | N/A | 100 |
| 1616121 | CATACCCAGACACAAACG | 48432 | 48449 | N/A | N/A | 101 |
| 1616122 | ATACCCAGACACAAACGG | 48431 | 48448 | N/A | N/A | 102 |
| 1616123 | CTACCCACCAACTCATCC | 48610 | 48627 | N/A | N/A | 103 |
| 1616124 | TACCCAGACACAAACGGC | 48430 | 48447 | N/A | N/A | 104 |
| 1616125 | ACCCAGACACAAACGGCC | 48429 | 48446 | N/A | N/A | 105 |
| 1616126 | CCCAGACACAAACGGCCC | 48428 | 48445 | N/A | N/A | 106 |
| 1616127 | CCAGACACAAACGGCCCA | 48427 | 48444 | N/A | N/A | 107 |
| 1616128 | CAGACACAAACGGCCCAA | 48426 | 48443 | N/A | N/A | 108 |
| 1616129 | AGACACAAACGGCCCAAT | 48425 | 48442 | N/A | N/A | 109 |
| 1616130 | GACACAAACGGCCCAATC | 48424 | 48441 | N/A | N/A | 110 |
| 1616131 | ACACAAACGGCCCAATCC | 48423 | 48440 | N/A | N/A | 111 |
| 1616132 | CACAAACGGCCCAATCCT | 48422 | 48439 | N/A | N/A | 112 |
| 1616133 | ACAAACGGCCCAATCCTG | 48421 | 48438 | N/A | N/A | 113 |
| 1616134 | CAAACGGCCCAATCCTGA | 48420 | 48437 | N/A | N/A | 114 |
| 1616135 | AAACGGCCCAATCCTGAG | 48419 | 48436 | N/A | N/A | 115 |
| 1616136 | AACGGCCCAATCCTGAGT | 48418 | 48435 | N/A | N/A | 116 |
| 1616137 | ACGGCCCAATCCTGAGTG | 48417 | 48434 | N/A | N/A | 117 |
| 1616138 | CGGCCCAATCCTGAGTGG | 48416 | 48433 | N/A | N/A | 118 |
| 1616139 | GGCCCAATCCTGAGTGGT | 48415 | 48432 | N/A | N/A | 119 |
| 1616140 | GCCCAATCCTGAGTGGTT | 48414 | 48431 | N/A | N/A | 120 |
| 1616141 | CCCAATCCTGAGTGGTTA | 48413 | 48430 | N/A | N/A | 121 |
| 1616142 | CCAATCCTGAGTGGTTAG | 48412 | 48429 | N/A | N/A | 122 |
| 1616143 | CAATCCTGAGTGGTTAGG | 48411 | 48428 | N/A | N/A | 123 |
| 1616144 | ACCCACCAACTCATCCAT | 48608 | 48625 | N/A | N/A | 124 |
| 1616145 | AATCCTGAGTGGTTAGGG | 48410 | 48427 | N/A | N/A | 125 |
| 1616146 | ATCCTGAGTGGTTAGGGC | 48409 | 48426 | N/A | N/A | 126 |
| 1616147 | TCCTGAGTGGTTAGGGCT | 48408 | 48425 | N/A | N/A | 127 |
| 1616148 | CCTGAGTGGTTAGGGCTG | 48407 | 48424 | N/A | N/A | 128 |
| 1616149 | CTGAGTGGTTAGGGCTGG | 48406 | 48423 | N/A | N/A | 129 |
| 1616150 | TGAGTGGTTAGGGCTGGA | 48405 | 48422 | N/A | N/A | 130 |
| 1616151 | GAGTGGTTAGGGCTGGAA | 48404 | 48421 | N/A | N/A | 131 |
| 1616152 | AGTGGTTAGGGCTGGAAT | 48403 | 48420 | N/A | N/A | 132 |
| 1616153 | GTGGTTAGGGCTGGAATA | 48402 | 48419 | N/A | N/A | 133 |
| 1616154 | TGGTTAGGGCTGGAATAG | 48401 | 48418 | N/A | N/A | 134 |
| 1616155 | GGTTAGGGCTGGAATAGA | 48400 | 48417 | N/A | N/A | 135 |
| 1616156 | GTTAGGGCTGGAATAGAA | 48399 | 48416 | N/A | N/A | 136 |
| 1616157 | TTAGGGCTGGAATAGAAG | 48398 | 48415 | N/A | N/A | 137 |
| 1616158 | TAGGGCTGGAATAGAAGG | 48397 | 48414 | N/A | N/A | 138 |
| 1616159 | AGGGCTGGAATAGAAGGA | 48396 | 48413 | N/A | N/A | 139 |
| 1616160 | GGGCTGGAATAGAAGGAA | 48395 | 48412 | N/A | N/A | 140 |
| 1616161 | GGCTGGAATAGAAGGAAG | 48394 | 48411 | N/A | N/A | 141 |
| 1616162 | GCTGGAATAGAAGGAAGA | 48393 | 48410 | N/A | N/A | 142 |
| 1616163 | CTGGAATAGAAGGAAGAA | 48392 | 48409 | N/A | N/A | 143 |
| 1616164 | TGGAATAGAAGGAAGAAC | 48391 | 48408 | N/A | N/A | 144 |
| 1616165 | CCACCAACTCATCCATCT | 48606 | 48623 | N/A | N/A | 145 |
| 1616166 | GGAATAGAAGGAAGAACC | 48390 | 48407 | N/A | N/A | 146 |
| 1616167 | GAATAGAAGGAAGAACCT | 48389 | 48406 | N/A | N/A | 147 |
| 1616168 | AATAGAAGGAAGAACCTG | 48388 | 48405 | N/A | N/A | 148 |
| 1616169 | ATAGAAGGAAGAACCTGA | 48387 | 48404 | N/A | N/A | 149 |
| 1616170 | TAGAAGGAAGAACCTGAT | 48386 | 48403 | N/A | N/A | 150 |
| 1616171 | AGAAGGAAGAACCTGATG | 48385 | 48402 | N/A | N/A | 151 |
| 1616172 | GAAGGAAGAACCTGATGA | 48384 | 48401 | N/A | N/A | 152 |
| 1616173 | AGGAAGAACCTGATGATG | 48382 | 48399 | N/A | N/A | 153 |
| 1616174 | GAAGAACCTGATGATGAG | 48380 | 48397 | N/A | N/A | 154 |
| 1616175 | AGAACCTGATGATGAGTA | 48378 | 48395 | N/A | N/A | 155 |
| 1616176 | AACCTGATGATGAGTAGT | 48376 | 48393 | N/A | N/A | 156 |
| 1616177 | CCTGATGATGAGTAGTGA | 48374 | 48391 | N/A | N/A | 157 |
| 1616178 | TGATGATGAGTAGTGAGA | 48372 | 48389 | N/A | N/A | 158 |
| 1616179 | ACCAACTCATCCATCTAT | 48604 | 48621 | N/A | N/A | 159 |
| 1616180 | ATGATGAGTAGTGAGAGT | 48370 | 48387 | N/A | N/A | 160 |
| 1616181 | GATGAGTAGTGAGAGTCA | 48368 | 48385 | N/A | N/A | 161 |
| 1616182 | TGAGTAGTGAGAGTCAAC | 48366 | 48383 | N/A | N/A | 162 |
| 1616183 | AGTAGTGAGAGTCAACCT | 48364 | 48381 | N/A | N/A | 163 |
| 1616184 | TAGTGAGAGTCAACCTGG | 48362 | 48379 | N/A | N/A | 164 |
| 1616185 | GTGAGAGTCAACCTGGAG | 48360 | 48377 | N/A | N/A | 165 |
| 1616186 | GAGAGTCAACCTGGAGGC | 48358 | 48375 | N/A | N/A | 166 |
| 1616187 | GAGTCAACCTGGAGGCCA | 48356 | 48373 | N/A | N/A | 167 |
| 1616188 | GTCAACCTGGAGGCCAAG | 48354 | 48371 | N/A | N/A | 168 |
| 1616189 | CAACCTGGAGGCCAAGGT | 48352 | 48369 | N/A | N/A | 169 |
| 1616190 | CAACTCATCCATCTATCC | 48602 | 48619 | N/A | N/A | 170 |
| 1616191 | ACCTGGAGGCCAAGGTGG | 48350 | 48367 | N/A | N/A | 171 |
| 1616192 | CTGGAGGCCAAGGTGGGC | 48348 | 48365 | N/A | N/A | 172 |
| 1616193 | GGAGGCCAAGGTGGGCTT | 48346 | 48363 | N/A | N/A | 173 |
| 1616194 | AGGCCAAGGTGGGCTTCC | 48344 | 48361 | N/A | N/A | 174 |
| 1616195 | GCCAAGGTGGGCTTCCCA | 48342 | 48359 | N/A | N/A | 175 |
| 1616196 | CAAGGTGGGCTTCCCAGA | 48340 | 48357 | N/A | N/A | 176 |
| 1616197 | AGGTGGGCTTCCCAGAGG | 48338 | 48355 | N/A | N/A | 177 |
| 1616198 | GTGGGCTTCCCAGAGGAG | 48336 | 48353 | N/A | N/A | 178 |
| 1616199 | GGGCTTCCCAGAGGAGGT | 48334 | 48351 | N/A | N/A | 179 |
| 1616200 | GCTTCCCAGAGGAGGTGA | 48332 | 48349 | N/A | N/A | 180 |
| 1616201 | ACTCATCCATCTATCCAT | 48600 | 48617 | N/A | N/A | 181 |
| 1616202 | TTCCCAGAGGAGGTGACC | 48330 | 48347 | N/A | N/A | 182 |
| 1616203 | CCCAGAGGAGGTGACCCT | 48328 | 48345 | N/A | N/A | 183 |
| 1616204 | CAGAGGAGGTGACCCTGA | 48326 | 48343 | N/A | N/A | 184 |
| 1616205 | GAGGAGGTGACCCTGAAT | 48324 | 48341 | N/A | N/A | 185 |
| 1616206 | GGAGGTGACCCTGAATCT | 48322 | 48339 | N/A | N/A | 186 |
| 1616207 | AGGTGACCCTGAATCTGG | 48320 | 48337 | N/A | N/A | 187 |
| 1616208 | GTGACCCTGAATCTGGAC | 48318 | 48335 | N/A | N/A | 188 |
| 1616209 | GACCCTGAATCTGGACTT | 48316 | 48333 | N/A | N/A | 189 |
| 1616210 | CCCTGAATCTGGACTTTG | 48314 | 48331 | N/A | N/A | 190 |
| 1616211 | CTGAATCTGGACTTTGAT | 48312 | 48329 | N/A | N/A | 191 |
| 1616212 | TCATCCATCTATCCATCC | 48598 | 48615 | N/A | N/A | 192 |
| 1616213 | GAATCTGGACTTTGATGG | 48310 | 48327 | N/A | N/A | 193 |
| 1616214 | ATCTGGACTTTGATGGAT | 48308 | 48325 | N/A | N/A | 194 |
| 1616215 | CTGGACTTTGATGGATAG | 48306 | 48323 | N/A | N/A | 195 |
| 1616216 | GGACTTTGATGGATAGGG | 48304 | 48321 | N/A | N/A | 196 |
| 1616217 | ACTTTGATGGATAGGGAG | 48302 | 48319 | N/A | N/A | 197 |
| 1616218 | TTTGATGGATAGGGAGGA | 48300 | 48317 | N/A | N/A | 198 |
| 1616219 | TGATGGATAGGGAGGAGT | 48298 | 48315 | N/A | N/A | 199 |
| 1616220 | ATGGATAGGGAGGAGTTT | 48296 | 48313 | N/A | N/A | 200 |
| 1616221 | GGATAGGGAGGAGTTTTC | 48294 | 48311 | N/A | N/A | 201 |
| 1616222 | ATAGGGAGGAGTTTTCCA | 48292 | 48309 | N/A | N/A | 202 |
| 1616223 | ATCCATCTATCCATCCAT | 48596 | 48613 | N/A | N/A | 203 |
| 1616224 | AGGGAGGAGTTTTCCAGG | 48290 | 48307 | N/A | N/A | 204 |
| 1616225 | GGAGGAGTTTTCCAGGTA | 48288 | 48305 | N/A | N/A | 205 |
| 1616226 | AGGAGTTTTCCAGGTAAA | 48286 | 48303 | N/A | N/A | 206 |
| 1616227 | GAGTTTTCCAGGTAAAGG | 48284 | 48301 | N/A | N/A | 207 |
| 1616228 | GTTTTCCAGGTAAAGGGA | 48282 | 48299 | N/A | N/A | 208 |
| 1616229 | TTTCCAGGTAAAGGGAAT | 48280 | 48297 | N/A | N/A | 209 |
| 1616230 | TCCAGGTAAAGGGAATGG | 48278 | 48295 | N/A | N/A | 210 |
| 1616231 | CAGGTAAAGGGAATGGCA | 48276 | 48293 | N/A | N/A | 211 |
| 1616232 | GGTAAAGGGAATGGCATG | 48274 | 48291 | N/A | N/A | 212 |
| 1616233 | TAAAGGGAATGGCATGGC | 48272 | 48289 | N/A | N/A | 213 |
| 1616234 | CCATCTATCCATCCATGT | 48594 | 48611 | N/A | N/A | 214 |
| 1616235 | AAGGGAATGGCATGGCGG | 48270 | 48287 | N/A | N/A | 215 |
| 1616236 | GGGAATGGCATGGCGGGC | 48268 | 48285 | N/A | N/A | 216 |
| 1616237 | GAATGGCATGGCGGGCAG | 48266 | 48283 | N/A | N/A | 217 |
| 1616238 | ATGGCATGGCGGGCAGTC | 48264 | 48281 | N/A | N/A | 218 |
| 1616239 | GGCATGGCGGGCAGTCTC | 48262 | 48279 | N/A | N/A | 219 |
| 1616240 | CATGGCGGGCAGTCTCAG | 48260 | 48277 | N/A | N/A | 220 |
| 1616241 | TGGCGGGCAGTCTCAGAG | 48258 | 48275 | N/A | N/A | 221 |
| 1616242 | GCGGGCAGTCTCAGAGGG | 48256 | 48273 | N/A | N/A | 222 |
| 1616243 | GGGCAGTCTCAGAGGGCC | 48254 | 48271 | N/A | N/A | 223 |
| 1616244 | GCAGTCTCAGAGGGCCAG | 48252 | 48269 | N/A | N/A | 224 |
| 1616245 | ATCTATCCATCCATGTAC | 48592 | 48609 | N/A | N/A | 225 |
| 1616246 | AGTCTCAGAGGGCCAGGA | 48250 | 48267 | N/A | N/A | 226 |
| 1616247 | TCTCAGAGGGCCAGGAGA | 48248 | 48265 | N/A | N/A | 227 |
| 1616248 | TCAGAGGGCCAGGAGAGT | 48246 | 48263 | N/A | N/A | 228 |
| 1616249 | AGAGGGCCAGGAGAGTGT | 48244 | 48261 | N/A | N/A | 229 |
| 1616250 | AGGGCCAGGAGAGTGTGG | 48242 | 48259 | N/A | N/A | 230 |
| 1616251 | GGCCAGGAGAGTGTGGAT | 48240 | 48257 | N/A | N/A | 231 |
| 1616252 | CCAGGAGAGTGTGGATGG | 48238 | 48255 | N/A | N/A | 232 |
| 1616253 | AGGAGAGTGTGGATGGTG | 48236 | 48253 | N/A | N/A | 233 |
| 1616254 | GAGAGTGTGGATGGTGTG | 48234 | 48251 | N/A | N/A | 234 |
| 1616255 | GAGTGTGGATGGTGTGGC | 48232 | 48249 | N/A | N/A | 235 |
| 1616256 | CCATCTAGCCACGAATCT | 48626 | 48643 | N/A | N/A | 236 |
| 1616257 | CTATCCATCCATGTACTC | 48590 | 48607 | N/A | N/A | 237 |
| 1616258 | GTGTGGATGGTGTGGCCA | 48230 | 48247 | N/A | N/A | 238 |
| 1616259 | GTGGATGGTGTGGCCAGA | 48228 | 48245 | N/A | N/A | 239 |
| 1616260 | GGATGGTGTGGCCAGAAA | 48226 | 48243 | N/A | N/A | 240 |
| 1616261 | ATGGTGTGGCCAGAAAGA | 48224 | 48241 | N/A | N/A | 241 |
| 1616262 | GGTGTGGCCAGAAAGAGG | 48222 | 48239 | N/A | N/A | 242 |
| 1616263 | TGTGGCCAGAAAGAGGAA | 48220 | 48237 | N/A | N/A | 243 |
| 1616264 | TGGCCAGAAAGAGGAAGA | 48218 | 48235 | N/A | N/A | 244 |
| 1616265 | GCCAGAAAGAGGAAGAGC | 48216 | 48233 | N/A | N/A | 245 |
| 1616266 | CAGAAAGAGGAAGAGCTG | 48214 | 48231 | N/A | N/A | 246 |
| 1616267 | GAAAGAGGAAGAGCTGGG | 48212 | 48229 | N/A | N/A | 247 |
| 1616268 | ATCCATCCATGTACTCAC | 48588 | 48605 | N/A | N/A | 248 |
| 1616269 | AAGAGGAAGAGCTGGGCA | 48210 | 48227 | N/A | N/A | 249 |
| 1616270 | GAGGAAGAGCTGGGCAGG | 48208 | 48225 | N/A | N/A | 250 |
| 1616271 | GGAAGAGCTGGGCAGGCA | 48206 | 48223 | N/A | N/A | 251 |
| 1616272 | AAGAGCTGGGCAGGCAGG | 48204 | 48221 | N/A | N/A | 252 |
| 1616273 | GAGCTGGGCAGGCAGGAA | 48202 | 48219 | N/A | N/A | 253 |
| 1616274 | GCTGGGCAGGCAGGAATG | 48200 | 48217 | N/A | N/A | 254 |
| 1616275 | TGGGCAGGCAGGAATGGT | 48198 | 48215 | N/A | N/A | 255 |
| 1616276 | GGCAGGCAGGAATGGTGA | 48196 | 48213 | N/A | N/A | 256 |
| 1616277 | CAGGCAGGAATGGTGAGT | 48194 | 48211 | N/A | N/A | 257 |
| 1616278 | GGCAGGAATGGTGAGTGG | 48192 | 48209 | N/A | N/A | 258 |
| 1616279 | CCATCCATGTACTCACCC | 48586 | 48603 | N/A | N/A | 259 |
| 1616280 | CAGGAATGGTGAGTGGAA | 48190 | 48207 | N/A | N/A | 260 |
| 1616281 | GGAATGGTGAGTGGAAGT | 48188 | 48205 | N/A | N/A | 261 |
| 1616282 | AATGGTGAGTGGAAGTGG | 48186 | 48203 | N/A | N/A | 262 |
| 1616283 | TGGTGAGTGGAAGTGGCA | 48184 | 48201 | N/A | N/A | 263 |
| 1616284 | GTGAGTGGAAGTGGCATG | 48182 | 48199 | N/A | N/A | 264 |
| 1616285 | GAGTGGAAGTGGCATGGG | 48180 | 48197 | N/A | N/A | 265 |
| 1616286 | GTGGAAGTGGCATGGGAG | 48178 | 48195 | N/A | N/A | 266 |
| 1616287 | GGAAGTGGCATGGGAGGG | 48176 | 48193 | N/A | N/A | 267 |
| 1616288 | AAGTGGCATGGGAGGGGC | 48174 | 48191 | N/A | N/A | 268 |
| 1616289 | GTGGCATGGGAGGGGCCG | 48172 | 48189 | N/A | N/A | 269 |
| 1616290 | ATCCATGTACTCACCCAT | 48584 | 48601 | N/A | N/A | 270 |
| 1616291 | GGCATGGGAGGGGCCGAG | 48170 | 48187 | N/A | N/A | 271 |
| 1616292 | CATGGGAGGGGCCGAGCA | 48168 | 48185 | N/A | N/A | 272 |
| 1616293 | TGGGAGGGGCCGAGCAAT | 48166 | 48183 | N/A | N/A | 273 |
| 1616294 | GGAGGGGCCGAGCAATGA | 48164 | 48181 | N/A | N/A | 274 |
| 1616295 | AGGGGCCGAGCAATGACC | 48162 | 48179 | N/A | N/A | 275 |
| 1616296 | GGGCCGAGCAATGACCCT | 48160 | 48177 | N/A | N/A | 276 |
| 1616297 | GCCGAGCAATGACCCTCA | 48158 | 48175 | N/A | N/A | 277 |
| 1616298 | CGAGCAATGACCCTCACC | 48156 | 48173 | N/A | N/A | 278 |
| 1616299 | AGCAATGACCCTCACCTC | 48154 | 48171 | N/A | N/A | 279 |
| 1616300 | CAATGACCCTCACCTCAT | 48152 | 48169 | N/A | N/A | 280 |
| 1616301 | CCATGTACTCACCCATCT | 48582 | 48599 | N/A | N/A | 281 |
| 1616302 | ATGACCCTCACCTCATGC | 48150 | 48167 | N/A | N/A | 282 |
| 1616303 | GACCCTCACCTCATGCAG | 48148 | 48165 | N/A | N/A | 283 |
| 1616304 | CCCTCACCTCATGCAGAC | 48146 | 48163 | N/A | N/A | 284 |
| 1616305 | CTCACCTCATGCAGACAG | 48144 | 48161 | N/A | N/A | 285 |
| 1616306 | CACCTCATGCAGACAGGT | 48142 | 48159 | N/A | N/A | 286 |
| 1616307 | CCTCATGCAGACAGGTGT | 48140 | 48157 | N/A | N/A | 287 |
| 1616308 | TCATGCAGACAGGTGTAC | 48138 | 48155 | 2454 | 2471 | 288 |
| 1616309 | ATGCAGACAGGTGTACTG | 48136 | 48153 | 2452 | 2469 | 289 |
| 1616310 | GCAGACAGGTGTACTGGA | 48134 | 48151 | 2450 | 2467 | 290 |
| 1616311 | AGACAGGTGTACTGGACA | 48132 | 48149 | 2448 | 2465 | 291 |
| 1616312 | ATGTACTCACCCATCTCT | 48580 | 48597 | N/A | N/A | 292 |
| 1616313 | ACAGGTGTACTGGACATG | 48130 | 48147 | 2446 | 2463 | 293 |
| 1616314 | AGGTGTACTGGACATGGT | 48128 | 48145 | 2444 | 2461 | 294 |
| 1616315 | GTGTACTGGACATGGTAC | 48126 | 48143 | 2442 | 2459 | 295 |
| 1616316 | GTACTGGACATGGTACGG | 48124 | 48141 | 2440 | 2457 | 296 |
| 1616317 | ACTGGACATGGTACGGGG | 48122 | 48139 | 2438 | 2455 | 297 |
| 1616318 | TGGACATGGTACGGGGCC | 48120 | 48137 | 2436 | 2453 | 298 |
| 1616319 | GACATGGTACGGGGCCAC | 48118 | 48135 | 2434 | 2451 | 299 |
| 1616320 | CATGGTACGGGGCCACCT | 48116 | 48133 | 2432 | 2449 | 300 |
| 1616321 | TGGTACGGGGCCACCTTC | 48114 | 48131 | 2430 | 2447 | 301 |
| 1616322 | GTACGGGGCCACCTTCTC | 48112 | 48129 | 2428 | 2445 | 302 |
| 1616323 | GTACTCACCCATCTCTCC | 48578 | 48595 | N/A | N/A | 303 |
| 1616324 | ACGGGGCCACCTTCTCCT | 48110 | 48127 | 2426 | 2443 | 304 |
| 1616325 | GGGGCCACCTTCTCCTCG | 48108 | 48125 | 2424 | 2441 | 305 |
| 1616326 | GGCCACCTTCTCCTCGCC | 48106 | 48123 | 2422 | 2439 | 306 |
| 1616327 | CCACCTTCTCCTCGCCTT | 48104 | 48121 | 2420 | 2437 | 307 |
| 1616328 | ACTCACCCATCTCTCCAT | 48576 | 48593 | N/A | N/A | 308 |
| 1616329 | TCACCCATCTCTCCATCC | 48574 | 48591 | N/A | N/A | 309 |
| 1616330 | ACCCATCTCTCCATCCAT | 48572 | 48589 | N/A | N/A | 310 |
| 1616331 | ATCTAGCCACGAATCTAC | 48624 | 48641 | N/A | N/A | 311 |
| 1616332 | CCATCTCTCCATCCATCC | 48570 | 48587 | N/A | N/A | 312 |
| 1616333 | ATCTCTCCATCCATCCTT | 48568 | 48585 | N/A | N/A | 313 |
| 1616334 | CTCTCCATCCATCCTTTT | 48566 | 48583 | N/A | N/A | 314 |
| 1616335 | CTCCATCCATCCTTTTAT | 48564 | 48581 | N/A | N/A | 315 |
| 1616336 | CCATCCATCCTTTTATCT | 48562 | 48579 | N/A | N/A | 316 |
| 1616337 | ATCCATCCTTTTATCTAC | 48560 | 48577 | N/A | N/A | 317 |
| 1616338 | CCATCCTTTTATCTACTC | 48558 | 48575 | N/A | N/A | 318 |
| 1616339 | ATCCTTTTATCTACTCAT | 48556 | 48573 | N/A | N/A | 319 |
| 1616340 | CCTTTTATCTACTCATCA | 48554 | 48571 | N/A | N/A | 320 |
| 1616341 | TTTTATCTACTCATCACT | 48552 | 48569 | N/A | N/A | 321 |
| 1616342 | CTAGCCACGAATCTACCC | 48622 | 48639 | N/A | N/A | 322 |
| 1616343 | TTATCTACTCATCACTCA | 48550 | 48567 | N/A | N/A | 323 |
| 1616344 | ATCTACTCATCACTCATT | 48548 | 48565 | N/A | N/A | 324 |
| 1616345 | CTACTCATCACTCATTCA | 48546 | 48563 | N/A | N/A | 325 |
| 1616346 | ACTCATCACTCATTCATC | 48544 | 48561 | N/A | N/A | 326 |
| 1616347 | TCATCACTCATTCATCTG | 48542 | 48559 | N/A | N/A | 327 |
| 1616348 | ATCACTCATTCATCTGTT | 48540 | 48557 | N/A | N/A | 328 |
| 1616349 | CACTCATTCATCTGTTCA | 48538 | 48555 | N/A | N/A | 329 |
| 1616350 | CTCATTCATCTGTTCAAT | 48536 | 48553 | N/A | N/A | 330 |
| 1616351 | CATTCATCTGTTCAATCA | 48534 | 48551 | N/A | N/A | 331 |
| 1616352 | TTCATCTGTTCAATCATT | 48532 | 48549 | N/A | N/A | 332 |

### Example 2: Effect of Uniform MOE Modified Oligonucleotides with Uniform Phosphorothioate Internucleoside Linkages on Human UNC13A RNA In Vitro, Single Dose

Modified oligonucleotides complementary to human UNC13A nucleic acid (described herein above) are tested for their single dose effects on UNC13A RNA in vitro in cultured cells that express UNC13A.

The cultured cells are treated with the modified oligonucleotides and RNA is extracted for quantitative real time RTPCR analysis of UNC13A RNA. Primer-probe set A is used to determine the amount of UNC13A RNA that includes the CE20x cryptic exon. Primer probe B is used to determine the amount of UNC13A RNA that excludes the CE20x cryptic exon. Primer probe set C is used to determine the amount of total UNC13A.

Modified oligonucleotides are found to reduce the amount of UNC13A RNA that includes CE20x. Modified oligonucleotides are found to increase the amount of UNC13A RNA that excludes CE20x. Modified oligonucleotides are found to increase the amount of total UNC13A RNA.

### Example 3: Activity of Modified Oligonucleotides Complementary to Human UNC13A RNA

Modified oligonucleotides complementary to a human UNC13A RNA were tested for their single dose effects on UNC13A RNA in vitro. The modified oligonucleotides were tested in a series of experiments that had the following culture conditions.

Cultured SH-SY5Y cells were treated with 100 μM cycloheximide for 24 hours prior to treatment with modified oligonucleotide. The SH-SY5Y cells were then treated with modified oligonucleotide at a concentration of 7,000 nM by electroporation at a density of 100,000 cells per well, and plated back into media containing cycloheximide. After a treatment period of approximately 24 hours, total RNA was isolated from the cells and UNC13A RNA levels were measured by quantitative real-time RTPCR.

UNCA13A RNA levels were measured by human primer-probe set RTS54362, designed to specifically recognize cryptic exon 1, thus measuring RNA that includes cryptic exon 1 (forward sequence GTACAACCTGGACAAGCGAACT, designated herein as SEQ ID NO: 6; reverse sequence GGAAACCCAGGCAGCTCAT, designated herein as SEQ ID NO: 7; probe sequence ATCAAAGGCGAGGAGAAGGTGGC, designated herein as SEQ ID NO: 8), as indicated in the tables below.

UNCA13A RNA levels were also measured by human primer-probe set RTS54363, designed to recognize all three cryptic exons thus measuring RNA that includes any of the three cryptic exons between exons 20 and 21 of UNC13A (complete cryptic exon inclusion) (forward sequence TGGATGGAGAGATGGAACCT, designated herein as SEQ ID NO: 9; reverse sequence GGGCTGTCTCATCGTAGTAAAC, designated herein as SEQ ID NO: 10; probe sequence TTGGCATCTGGGATCTTCACGACC, designated herein as SEQ ID NO: 11), as indicated in the tables below.

UNCA13A RNA levels were also measured by human primer-probe set RTS54365, designed to specifically detect complete cryptic exon exclusion (“exclusion transcripts”), thus measuring RNA that does not include cryptic exon 1, cryptic exon 2, and cryptic exon 3 between exons 20 and 21 of UNC13A (forward sequence CACCTGTCTGCATGAGAACCT, designated herein as SEQ ID NO: 12; reverse sequence CATGGCAAACTCGTCCACAATC, designated herein as SEQ ID NO: 13; probe sequence TAAACCTTCCAGGCATCGTCACCC, designated herein as SEQ ID NO: 14), as indicated in the tables below. The inherent expression of transcripts that exclude all three cryptic exons (“exclusion transcripts”) recognized by RTS54365 is significantly higher than transcripts recognized by RTS54362 or RTS54363 (“inclusion transcripts”), Therefore, levels of exclusion transcript are expected to remain unchanged or only slightly increase even when levels of inclusion transcripts decrease.

UNC13A RNA levels were normalized to total RNA content, as measured by RIBOGREEN®. Reduction of UNC13A RNA is presented in the table below as percent UNC13A RNA relative to the amount of UNC13A RNA in untreated control cells (% UTC). The values marked with a “T” indicate that the modified oligonucleotide is complementary to the amplicon region of the primer probe set. Additional assays may be used to measure the potency and efficacy of the modified oligonucleotides complementary to the amplicon region.

TABLE 2
Effect of modified oligonucleotides
on human UNC13A RNA in SH-SY5Y cells

| Compound | UNC13A RNA (% UTC) | | |
|---|---|---|---|
| No. | RTS54362 | RTS54363 | RTS54365 |
| 1616241 | 61 | 93 | 72 |
| 1616242 | 40 | 89 | 70 |
| 1616243 | 2 | 65 | 21 |
| 1616244 | 100 | 114 | 96 |
| 1616246 | 36 | 130 | 83 |
| 1616247 | 86 | 114 | 96 |
| 1616248 | 72 | 135 | 76 |
| 1616249 | 78 | 157 | 93 |
| 1616250 | 34 | 82 | 53 |
| 1616251 | 81 | 65 | 75 |
| 1616252 | 80 | 102 | 102 |
| 1616253 | 122 | 118 | 95 |
| 1616254 | 216 | 99 | 92 |
| 1616255 | 219 | 121 | 70 |
| 1616258 | 547 | 104 | 70 |
| 1616259 | 162 | 138 | 82 |
| 1616260 | 110 | 116 | 69 |
| 1616261 | 70 | 133 | 88 |
| 1616262 | 94 | 113 | 99 |
| 1616263 | 103 | 111 | 101 |
| 1616264 | 110 | 146 | 109 |
| 1616265 | 48 | 115 | 105 |
| 1616266 | 135 | 103 | 80 |
| 1616267 | 66 | 97 | 68 |
| 1616269 | 79 | 107 | 90 |
| 1616270 | 55 | 78 | 59 |
| 1616271 | 80 | 108 | 93 |
| 1616272 | 234 | 88 | 84 |
| 1616273 | 239 | 92 | 75 |
| 1616274 | 136 | 81 | 73 |
| 1616275 | 180 | 65 | 72 |
| 1616276 | 358 | 106 | 85 |
| 1616277 | 79 | 116 | 93 |
| 1616278 | 78 | 104 | 95 |
| 1616280 | 90 | 117 | 69 |
| 1616281 | 65 | 129 | 103 |
| 1616282 | 75 | 115 | 98 |
| 1616283 | 43 | 111 | 70 |
| 1616284 | 153 | 97 | 92 |
| 1616285 | 95 | 115 | 110 |
| 1616286 | 45 | 77 | 71 |
| 1616287 | 59 | 91 | 81 |
| 1616288 | 110 | 128 | 103 |
| 1616289 | 140 | 106 | 97 |
| 1616291 | 22 | 108 | 81 |
| 1616292 | 73 | 94 | 82 |
| 1616293 | 180 | 110 | 99 |
| 1616294 | 102 | 126 | 95 |
| 1616295 | 64 | 94 | 80 |
| 1616296 | 79 | 120 | 104 |
| 1616297 | 89 | 124 | 107 |
| 1616298 | 62† | 171 | 74† |
| 1616299 | 69† | 109 | 67† |
| 1616300 | 61† | 91 | 92† |
| 1616302 | 92† | 113 | 120† |
| 1616303 | 67† | 87 | 98† |
| 1616304 | 102† | 95 | 82† |
| 1616305 | 205† | 108 | 64† |
| 1616306 | 35† | 106 | 58† |
| 1616307 | 141† | 102 | 58† |
| 1616308 | 61† | 138 | 18† |
| 1616309 | 59† | 146 | 23† |
| 1616310 | 18† | 174 | 52† |
| 1616311 | 31† | 117 | 95† |
| 1616313 | 29† | 106 | 84† |
| 1616314 | 7† | 152 | 89† |
| 1616315 | 64† | 126 | 112† |
| 1616316 | 48† | 122 | 124† |
| 1616317 | 74† | 215 | 80 |
| 1616318 | 15† | 106 | 101 |
| 1616319 | 28† | 112 | 110 |
| 1616320 | 12† | 110 | 136 |
| 1616321 | 16† | 125 | 108 |
| 1616322 | 16† | 104 | 63 |
| 1616324 | 2† | 58 | 96 |
| 1616325 | 1† | 74 | 62 |
| 1616326 | 0† | 83 | 55 |
| 1616327 | 1† | 77 | 58 |

TABLE 3
Effect of modified oligonucleotides
on human UNC13A RNA in SH-SY5Y cells

| Compound | UNC13A RNA (% UTC) | | |
|---|---|---|---|
| No. | RTS54362 | RTS54363 | RTS54365 |
| 1616156 | 76 | 89 | 109 |
| 1616157 | 113 | 110 | 106 |
| 1616158 | 42 | 136 | 113 |
| 1616159 | 111 | 84 | 93 |
| 1616160 | 95 | 93 | 87 |
| 1616161 | 76 | 111 | 92 |
| 1616162 | 172 | 168 | 110 |
| 1616163 | 154 | 179 | 96 |
| 1616164 | 148 | 233 | 100 |
| 1616166 | 54 | 172 | 119 |
| 1616167 | 138 | 163 | 103 |
| 1616168 | 133 | 146 | 104 |
| 1616169 | 158 | 164 | 99 |
| 1616170 | 168 | 113 | 101 |
| 1616171 | 150 | 150 | 87 |
| 1616172 | 130 | 156 | 106 |
| 1616173 | 260 | 225 | 109 |
| 1616174 | 188 | 172 | 83 |
| 1616175 | 282 | 245 | 99 |
| 1616176 | 257 | 252 | 106 |
| 1616177 | 143 | 188 | 100 |
| 1616178 | 208 | 207 | 88 |
| 1616180 | 133 | 153 | 100 |
| 1616181 | 138 | 143 | 124 |
| 1616182 | 114 | 154 | 96 |
| 1616183 | 180 | 197 | 123 |
| 1616184 | 77 | 170 | 70 |
| 1616185 | 110 | 170 | 96 |
| 1616186 | 141 | 184 | 89 |
| 1616187 | 179 | 171 | 102 |
| 1616188 | 112 | 124 | 118 |
| 1616189 | 135 | 90 | 100 |
| 1616191 | 71 | 139 | 118 |
| 1616192 | 79 | 143 | 59 |
| 1616193 | 112 | 123 | 107 |
| 1616194 | 150 | 124 | 131 |
| 1616195 | 100 | 163 | 99 |
| 1616196 | 131 | 98 | 103 |
| 1616197 | 74 | 130 | 85 |
| 1616198 | 105 | 93 | 103 |
| 1616199 | 69 | 86 | 102 |
| 1616200 | 136 | 104 | 101 |
| 1616202 | 93 | 95 | 100 |
| 1616203 | 123 | 109 | 103 |
| 1616204 | 103 | 180 | 92 |
| 1616205 | 89 | 93 | 104 |
| 1616206 | 117 | 136 | 109 |
| 1616207 | 110 | 140 | 113 |
| 1616208 | 83 | 109 | 106 |
| 1616209 | 108 | 114 | 92 |
| 1616210 | 128 | 170 | 128 |
| 1616211 | 172 | 224 | 102 |
| 1616213 | 189 | 246 | 84 |
| 1616214 | 171 | 312 | 93 |
| 1616215 | 290 | 206 | 94 |
| 1616216 | 128 | 181 | 114 |
| 1616217 | 136 | 91 | 106 |
| 1616218 | 101 | 98 | 85 |
| 1616219 | 120 | 106 | 104 |
| 1616220 | 71 | 103 | 97 |
| 1616221 | 106 | 92 | 100 |
| 1616222 | 89 | 96 | 115 |
| 1616224 | 80 | 75 | 130 |
| 1616225 | 130 | 69 | 93 |
| 1616226 | 93 | 111 | 108 |
| 1616227 | 121 | 101 | 116 |
| 1616228 | 129 | 122 | 116 |
| 1616229 | 91 | 114 | 86 |
| 1616230 | 125 | 109 | 100 |
| 1616231 | 116 | 99 | 89 |
| 1616232 | 136 | 92 | 96 |
| 1616233 | 117 | 120 | 99 |
| 1616235 | 284 | 91 | 91 |
| 1616236 | 7 | 14 | 19 |
| 1616237 | 224 | 63 | 78 |
| 1616238 | 132 | 92 | 88 |
| 1616239 | 67 | 85 | 108 |
| 1616240 | 88 | 78 | 56 |

TABLE 4
Effect of modified oligonucleotides
on human UNC13A RNA in SH-SY5Y cells

| Compound | UNC13A RNA (% UTC) | | |
|---|---|---|---|
| No. | RTS54362 | RTS54363 | RTS54365 |
| 1616048 | 79 | 26 | 93 |
| 1616049 | 40 | 23 | 95 |
| 1616050 | 57 | 18 | 83 |
| 1616077 | 48 | 19 | 105 |
| 1616078 | 37† | 14 | 97 |
| 1616079 | 28† | 6 | 99 |
| 1616080 | 224† | 15 | 100 |
| 1616082 | 29† | 12 | 103 |
| 1616083 | 51† | 23 | 98 |
| 1616084 | 97† | 26 | 90 |
| 1616085 | 56† | 16 | 111 |
| 1616086 | 69† | 26 | 102 |
| 1616087 | 98† | 39 | 94 |
| 1616088 | 60† | 51 | 105 |
| 1616089 | 97† | 49 | 97 |
| 1616090 | 51† | 61 | 94 |
| 1616091 | 42† | 54 | 89 |
| 1616092 | 23† | 68 | 90 |
| 1616093 | 33† | 56 | 102 |
| 1616094 | 7† | 83 | 89 |
| 1616095 | 32† | 64 | 88 |
| 1616096 | 53† | 85 | 93 |
| 1616097 | 106† | 84 | 81 |
| 1616098 | 175† | 78 | 86 |
| 1616099 | 59† | 79 | 80 |
| 1616100 | 76† | 69 | 88 |
| 1616101 | 99† | 100 | 94 |
| 1616103 | 63† | 82 | 92 |
| 1616104 | 55† | 57 | 101 |
| 1616105 | 69† | 48 | 94 |
| 1616106 | 61† | 38 | 84 |
| 1616107 | 63† | 39 | 88 |
| 1616108 | 41† | 35 | 93 |
| 1616109 | 58† | 42 | 103 |
| 1616110 | 23† | 31 | 96 |
| 1616111 | 46 | 26 | 95 |
| 1616112 | 50 | 34 | 90 |
| 1616113 | 98 | 30 | 89 |
| 1616114 | 19 | 28 | 100 |
| 1616115 | 68 | 37 | 95 |
| 1616116 | 23 | 32 | 88 |
| 1616117 | 50 | 30 | 95 |
| 1616118 | 74 | 31 | 95 |
| 1616119 | 25 | 32 | 104 |
| 1616120 | 31 | 30 | 96 |
| 1616121 | 46 | 46 | 101 |
| 1616122 | 225 | 128 | 101 |
| 1616124 | 319 | 198 | 95 |
| 1616125 | 357 | 227 | 91 |
| 1616126 | 390 | 208 | 90 |
| 1616127 | 353 | 241 | 90 |
| 1616128 | 166 | 130 | 106 |
| 1616129 | 102 | 79 | 98 |
| 1616130 | 99 | 70 | 89 |
| 1616131 | 81 | 90 | 83 |
| 1616132 | 79 | 68 | 112 |
| 1616133 | 146 | 101 | 95 |
| 1616134 | 76 | 88 | 98 |
| 1616135 | 95 | 103 | 98 |
| 1616136 | 76 | 80 | 94 |
| 1616137 | 29 | 83 | 91 |
| 1616138 | 39 | 90 | 95 |
| 1616139 | 107 | 97 | 92 |
| 1616140 | 96 | 110 | 104 |
| 1616141 | 84 | 81 | 83 |
| 1616142 | 144 | 95 | 116 |
| 1616143 | 75 | 101 | 96 |
| 1616145 | 94 | 67 | 92 |
| 1616146 | 44 | 81 | 104 |
| 1616147 | 100 | 84 | 94 |
| 1616148 | 200 | 88 | 108 |
| 1616149 | 91 | 93 | 121 |
| 1616150 | 185 | 89 | 104 |
| 1616151 | 33 | 61 | 126 |
| 1616152 | 114 | 77 | 102 |
| 1616153 | 53 | 62 | 87 |
| 1616154 | 14 | 79 | 106 |
| 1616155 | 96 | 72 | 95 |

TABLE 5
Effect of modified oligonucleotides
on human UNC13A RNA in SH-SY5Y cells

| Compound | UNC13A RNA (% UTC) | | |
|---|---|---|---|
| No. | RTS54362 | RTS54363 | RTS54365 |
| 1616041 | 71 | 54 | 93 |
| 1616042 | 138 | 33 | 98 |
| 1616043 | 51 | 31 | 86 |
| 1616044 | 60 | 33 | 96 |
| 1616045 | 35 | 7 | 87 |
| 1616046 | 62 | 12 | 99 |
| 1616047 | 63 | 36 | 92 |
| 1616051 | 119 | 133 | 100 |
| 1616052 | 52 | 26 | 87 |
| 1616053 | 36 | 24 | 77 |
| 1616054 | 65 | 21 | 101 |
| 1616055 | 75 | 25 | 89 |
| 1616056 | 83 | 11 | 90 |
| 1616057 | 44 | 15 | 93 |
| 1616058 | 49 | 19 | 92 |
| 1616059 | 73 | 36 | 85 |
| 1616060 | 53 | 11 | 114 |
| 1616061 | 73 | 27 | 102 |
| 1616062 | 185 | 202 | 88 |
| 1616063 | 40 | 38 | 82 |
| 1616064 | 66 | 32 | 94 |
| 1616065 | 41 | 9 | 82 |
| 1616066 | 44 | 19 | 95 |
| 1616067 | 43 | 11 | 89 |
| 1616068 | 41 | 31 | 98 |
| 1616069 | 33 | 23 | 81 |
| 1616070 | 46 | 34 | 92 |
| 1616071 | 73 | 11 | 85 |
| 1616072 | 78 | 36 | 158 |
| 1616073 | 87 | 146 | 107 |
| 1616074 | 160 | 24 | 115 |
| 1616075 | 92 | 44 | 118 |
| 1616076 | 79 | 44 | 78 |
| 1616081 | 202 | 323 | 98 |
| 1616102 | 135 | 226 | 71 |
| 1616123 | 155 | 112 | 91 |
| 1616144 | 147 | 111 | 79 |
| 1616165 | 175 | 286 | 93 |
| 1616179 | 254 | 94 | 78 |
| 1616190 | 168 | 213 | 97 |
| 1616201 | 131 | 106 | 75 |
| 1616212 | 172 | 78 | 91 |
| 1616223 | 95 | 75 | 66 |
| 1616234 | 130 | 50 | 70 |
| 1616245 | 100 | 25 | 106 |
| 1616256 | 197 | 207 | 80 |
| 1616257 | 69 | 16 | 92 |
| 1616268 | 59 | 17 | 88 |
| 1616279 | 69 | 14† | 91 |
| 1616290 | 63 | 23† | 86 |
| 1616301 | 56 | 19† | 92 |
| 1616312 | 79 | 49† | 88 |
| 1616323 | 90 | 39† | 92 |
| 1616328 | 48 | 36† | 106 |
| 1616329 | 47 | 57† | 88 |
| 1616330 | 89 | 31† | 87 |
| 1616331 | 94 | 110† | 84 |
| 1616332 | 139 | 18† | 109 |
| 1616333 | 69 | 34† | 100 |
| 1616334 | 90 | 5† | 91 |
| 1616335 | 77 | 23† | 108 |
| 1616336 | 85 | 26† | 86 |
| 1616337 | 29 | 8† | 88 |
| 1616338 | 49 | 23† | 85 |
| 1616339 | 56 | 12† | 93 |
| 1616340 | 51 | 29 | 94 |
| 1616341 | 57 | 69 | 97 |
| 1616342 | 161 | 196 | 87 |
| 1616343 | 71 | 158 | 82 |
| 1616344 | 86 | 109 | 86 |
| 1616345 | 105 | 71 | 96 |
| 1616346 | 70 | 18 | 81 |
| 1616347 | 57 | 23 | 92 |
| 1616348 | 79 | 29 | 93 |
| 1616349 | 55 | 32 | 92 |
| 1616350 | 36 | 40 | 85 |
| 1616351 | 32 | 5 | 88 |
| 1616352 | 68 | 31 | 93 |

1. An oligomeric compound comprising a modified oligonucleotide consisting of 8 to 80 linked nucleosides, wherein the nucleobase sequence of the modified oligonucleotide is at least 80% complementary to an equal length portion of a UNC13A nucleic acid, and wherein the modified oligonucleotide has at least one modification selected from a modified sugar moiety and a modified internucleoside linkage.

2. The oligomeric compound of claim 1, wherein the UNC13A nucleic acid has the nucleobase sequence of SEQ ID NO: 1 or SEQ ID NO: 2.

3. The oligomeric compound of claim 1 or claim 2, wherein the nucleobase sequence of the modified oligonucleotide is at least 80% complementary to an equal length portion within nucleobases 48,128-48,151, 48,432-48,465, or 48,466-48,561 of SEQ ID NO: 1.

4. The oligomeric compound of any of claims 1-3, wherein the nucleobase sequence of the modified oligonucleotide is at least 85%, at least 90%, at least 95%, or 100% complementary to an equal length portion of the UNC13A nucleic acid.

5. An oligomeric compound comprising a modified oligonucleotide consisting of 8 to 80 linked nucleosides, wherein the nucleobase sequence of the modified oligonucleotide comprises at least 8, at least 9, at least 10, at least 11, at least 12, at least 13, at least 14, at least 15, at least 16, at least 17, or 18 contiguous nucleobases of any of the nucleobase sequences of SEQ ID NOs: 21-332, and wherein the modified oligonucleotide comprises at least one modification selected from a modified sugar moiety and a modified internucleoside linkage.

6. The oligomeric compound of claim 5, wherein the nucleobase sequence of the modified oligonucleotide comprises the nucleobase sequence of any of SEQ ID NOs: 21-332.

7. The oligomeric compound of claim 5, wherein the modified oligonucleotide has a nucleobase sequence consisting of the nucleobase sequence of any of SEQ ID NOs: 21-332.

8. The oligomeric compound of any one of claims 5-7, wherein the modified oligonucleotide has a nucleobase sequence comprising at least 8, at least 9, at least 10, at least 11, at least 12, at least 13, at least 14, at least 15, at least 16, at least 17, or 18 contiguous nucleobases of any of the nucleobase sequences of:
SEQ ID NOs: 290, 291, 293, or 294;
SEQ ID NOs: 85-101; or
SEQ ID NOs: 21-30, 32-41, 43-52, 54-60, 62-66, or 326-332.

9. The oligomeric compound of any of claims 5-8, wherein the nucleobase sequence of the modified oligonucleotide is at least 85%, at least 90%, at least 95%, or 100% complementary to an equal length portion of a UNC13A nucleic acid, wherein the UNC13A nucleic acid has the nucleobase sequence of SEQ ID NO: 1 or SEQ ID NO: 2.

10. An oligomeric compound comprising a modified oligonucleotide consisting of 12 to 50 linked nucleosides, wherein the nucleobase sequence of the modified oligonucleotide comprises at least 8, at least 9, at least 10, at least 11, at least 12, at least 13, at least 14, at least 15, at least 16, at least 17, or 18 contiguous nucleobases complementary to:
48,128-48,151 of SEQ ID NO: 1;
48,432-48,465 of SEQ ID NO: 1; or
48,466-48,561 of SEQ ID NO: 1.

11. The oligomeric compound of any of claims 1-10, wherein the modified oligonucleotide consists of 10 to 25, 10 to 30, 10 to 50, 12 to 20, 12 to 25, 12 to 30, 12 to 50, 13 to 20, 13 to 25, 13 to 30, 13 to 50, 14 to 20, 14 to 25, 14 to 30, 14 to 50, 15 to 20, 15 to 25, 15 to 30, 15 to 50, 16 to 18, 16 to 20, 16 to 25, 16 to 30, 16 to 50, 17 to 20, 17 to 25, 17 to 30, 17 to 50, 18 to 20, 18 to 22, 18 to 25, 18 to 30, 18 to 50, 19 to 20, 19 to 25, 19 to 30, 19 to 50, 20 to 25, 20 to 30, 20 to 50, 21 to 25, 21 to 30, 21 to 50, 22 to 25, 22 to 30, 22 to 50, 23 to 25, 23 to 30, or 23 to 50 linked nucleosides.

12. The oligomeric compound of any of claims 1-11, wherein the modified oligonucleotide consists of 18 linked nucleosides.

13. The oligomeric compound of any of claims 1-12, wherein at least one nucleoside of the modified oligonucleotide comprises a modified sugar moiety.

14. The oligomeric compound of claim 13, wherein the modified sugar moiety comprises a bicyclic sugar moiety.

15. The oligomeric compound of claim 14, wherein the bicyclic sugar moiety comprises a 2′-4′ bridge selected from —O—CH₂—; and —O—CH(CH₃)—.

16. The oligomeric compound of claim 13, wherein the modified sugar moiety comprises a non-bicyclic modified sugar moiety.

17. The oligomeric compound of claim 16, wherein the non-bicyclic modified sugar moiety is a 2′-MOE sugar moiety, a 2′-OMe sugar moiety, a 2′-NMA sugar moiety, or a 2′-F sugar moiety.

18. The oligomeric compound of any of claims 1-17, wherein at least one nucleoside of the modified oligonucleotide compound comprises a sugar surrogate.

19. The oligomeric compound of any of claims 13-18, wherein each nucleoside of the modified oligonucleotide comprises a modified sugar moiety.

20. The oligomeric compound of claim 19, wherein each modified sugar moiety is a 2′-MOE sugar moiety.

21. The oligomeric compound of claim 19, wherein each modified sugar moiety is a 2′-NMA sugar moiety.

22. The oligomeric compound of any of claims 1-21, wherein the modified oligonucleotide comprises at least one modified internucleoside linkage.

23. The oligomeric compound of claim 22, wherein at least one modified internucleoside linkage is a phosphorothioate internucleoside linkage.

24. The oligomeric compound of claim 22 or claim 23, wherein each internucleoside linkage is a modified internucleoside linkage.

25. The oligomeric compound of claim 24, wherein each internucleoside linkage is a phosphorothioate internucleoside linkage.

26. The oligomeric compound of any of claims 22-23, wherein at least one internucleoside linkage of the modified oligonucleotide is a phosphodiester internucleoside linkage.

27. The oligomeric compound of any of claims 1-24 or 26, wherein each internucleoside linkage of the modified oligonucleotide is independently selected from a phosphodiester or a phosphorothioate internucleoside linkage.

28. The oligomeric compound of any of claims 1-23, or 26-27, wherein at least 10, at least 11, at least 12, at least 13, at least 14, at least 15, at least 16, or 17 internucleoside linkages of the modified oligonucleotide are phosphorothioate internucleoside linkages.

29. The oligomeric compound of any of claims 1-28, wherein the modified oligonucleotide comprises at least one modified nucleobase.

30. The oligomeric compound of claim 29, wherein the modified nucleobase is 5-methylcytosine.

31. The oligomeric compound of claim 30, wherein each cytosine is a 5-methylcytosine.

32. The oligomeric compound of any of claims 1-31, consisting of the modified oligonucleotide.

33. The oligomeric compound of any one of claims 1-32, wherein the modified oligonucleotide is a pharmaceutically acceptable salt thereof.

34. The oligomeric compound of claim 33, which is a pharmaceutically acceptable salt comprising one or more cations selected from sodium, potassium, calcium, and magnesium.

35. The oligomeric compound of any of claims 1-31, wherein the oligomeric compound comprises a conjugate group.

36. The oligomeric compound of claim 35, wherein the conjugate group comprises a conjugate linker and a conjugate moiety.

37. The oligomeric compound of claim 36, wherein the conjugate linker consists of a single bond.

38. The oligomeric compound of claim 36 or claim 37, wherein the conjugate linker is cleavable.

39. The oligomeric compound of any of claims 36-38, wherein the conjugate linker comprises 1-3 linker-nucleosides.

40. The oligomeric compound of any of claims 36-38, wherein the conjugate linker does not comprise any linker nucleosides.

41. The oligomeric compound of any of claims 35-40, wherein the conjugate group is attached to the modified oligonucleotide at the 5′-end of the modified oligonucleotide.

42. The oligomeric compound of any of claims 35-40, wherein the conjugate group is attached to the modified oligonucleotide at the 3′-end of the modified oligonucleotide.

43. The oligomeric compound of any of claims 1 to 42, wherein the oligomeric compound comprises a terminal group.

44. The oligomeric compound of claim 43 wherein the terminal group is an abasic sugar moiety.

45. The oligomeric compound of any one of claims 1-44 wherein the oligomeric compound is a singled-stranded oligomeric compound.

46. A chirally enriched population of oligomeric compounds of any of claims 1-45, wherein the population is enriched for modified oligonucleotides comprising at least one particular phosphorothioate internucleoside linkage having a particular stereochemical configuration.

47. The chirally enriched population of claim 46, wherein the population is enriched for modified oligonucleotides comprising at least one particular phosphorothioate internucleoside linkage having the (Sp) or (Rp) configuration.

48. The chirally enriched population of claim 46, wherein the population is enriched for modified oligonucleotides having a particular, independently selected stereochemical configuration at each phosphorothioate internucleoside linkage.

49. The chirally enriched population of claim 46, wherein the population is enriched for modified oligonucleotides having the (Rp) configuration at one particular phosphorothioate internucleoside linkage and the (Sp) configuration at each of the remaining phosphorothioate internucleoside linkages.

50. The chirally enriched population of claim 46, wherein the population is enriched for modified oligonucleotides having at least 3 contiguous phosphorothioate internucleoside linkages in the Sp, Sp, and Rp configurations, in the 5′ to 3′ direction.

51. A population of oligomeric compounds of any of claims 1-45, wherein all of the phosphorothioate internucleoside linkages of the modified oligonucleotide are stereorandom.

52. An oligomeric duplex, comprising a first oligomeric compound and a second oligomeric compound comprising a second modified oligonucleotide, wherein the first oligomeric compound is an oligomeric compound of any of claims 1-45.

53. The oligomeric duplex of claim 52, wherein the second modified oligonucleotide consists of 8 to 80 linked nucleosides, and wherein the second modified oligonucleotide comprises a complementary region of at least 8 nucleobases that is at least 90% complementary to an equal length portion of the first modified oligonucleotide.

54. An antisense agent comprising an antisense compound, wherein the antisense compound is the oligomeric compound of any of claims 1-45 or an oligomeric duplex of claim 52 or claim 53.

55. The antisense agent of claim 54, wherein the antisense agent is a splice-modulating agent capable of modulating splicing of UNC13A nucleic acid.

56. The antisense agent of claim 54 or claim 55, wherein the antisense agent comprises a conjugate group, wherein the conjugate group comprises a cell-targeting moiety.

57. A pharmaceutical composition comprising an oligomeric compound of any of claims 1-45, a population of any of claims 46-51, an oligomeric duplex of claim 52 or claim 53, or an antisense agent of any of claims 54-56, and a pharmaceutically acceptable diluent or carrier.

58. The pharmaceutical composition of claim 57, wherein the pharmaceutically acceptable diluent is phosphate-buffered saline or artificial cerebrospinal fluid.

59. The pharmaceutical composition of claim 58, wherein the pharmaceutical composition consists essentially of the oligomeric compound, the population, the oligomeric duplex, or the antisense agent, and phosphate-buffered saline or artificial cerebrospinal fluid.

60. A method comprising administering to a subject an oligomeric compound of any of claims 1-45, a population of any of claims 46-51, an oligomeric duplex of claim 52 or claim 53, an antisense agent of any of claims 54-56, or a pharmaceutical composition of any of claims 57-59.

61. A method of treating a disease associated with UNC13A comprising administering to a subject having or at risk for developing a disease associated with UNC13A a therapeutically effective amount of an oligomeric compound of any of claims 1-45, a population of any of claims 46-51, an oligomeric duplex of claim 52 or claim 53, an antisense agent of any of claims 54-56, or a pharmaceutical composition of any of claims 57-59, thereby treating the disease associated with UNC13A.

62. The method of claim 61, wherein the disease associated with UNC13A is a neurodegenerative disease.

63. The method of claim 62, wherein the neurodegenerative disease is amyotrophic lateral sclerosis (ALS) or frontotemporal dementia (FTD).

64. The method of claim 63, wherein at least one symptom of the neurodegenerative disease is ameliorated.

65. The method of claim 64, wherein the at least one symptom is motor dysfunction, muscle weakness, muscle wasting, synaptic dysfunction, fatigue, difficulty speaking, difficulty swallowing, shortness of breath, cognitive impairment, decreased longevity, or a combination thereof.

66. The method of claim 65, wherein administering an oligomeric compound of any of claims 1-45, a population of any of claims 46-51, an oligomeric duplex of claim 52 or claim 53, an antisense agent of any of claims 54-56, or a pharmaceutical composition of any of claims 57-59, improves motor function, improves muscle strength, increases muscle mass, improves speaking, improves swallowing, improves breathing, improves synaptic function, improves cognition, or increases longevity.

67. The method of any one of claims 60-66, wherein the subject is a human.

68. A method of increasing expression of UNC13A in a cell comprising contacting the cell with an oligomeric compound of any of claims 1-45, a population of any of claims 46-51, an oligomeric duplex of claim 52 or claim 53, an antisense agent of any of claims 54-56, or a pharmaceutical composition of any of claims 57-59.

69. A method of decreasing the amount of UNC13A RNA containing a cryptic exon in a cell comprising contacting the cell with an oligomeric compound of any of claims 1-45, a population of any of claims 46-51, an oligomeric duplex of claim 52 or claim 53, an antisense agent of any of claims 54-56, or a pharmaceutical composition of any of claims 57-59.

70. The method of claim 69, wherein the cryptic exon is between exons 20 and 21 of UNC13A.

71. The method of claim 70, wherein the cryptic exon is selected from CE-1, CE-2, and CE-3.

72. The method of any of claims 68-71, wherein the cell is a neuron or a glial cell, optionally wherein the cell is an astrocyte or microglial cell.

73. The method of any of claims 68-72, wherein the cell is a human cell.

74. Use of an oligomeric compound of any of claims 1-45, a population of any of claims 46-51, an oligomeric duplex of claim 52 or claim 53, an antisense agent of any of claims 54-56, or a pharmaceutical composition of any of claims 57-59 for treating a disease associated with UNC13A.

75. Use of an oligomeric compound of any of claims 1-45, a population of any of claims 46-51, an oligomeric duplex of claim 52 or claim 53, an antisense agent of any of claims 54-56, or a pharmaceutical composition of any of claims 57-59 in the manufacture of a medicament for treating a disease associated with UNC13A.

76. The use of claim 74 or claim 75, wherein the disease associated with UNC13A is a neurodegenerative disease.

77. The use of claim 76, wherein the neurodegenerative disease is amyotrophic lateral sclerosis (ALS) or frontotemporal dementia (FTD).
